(19) United States
(12) Patent Application Publication
(10) Pub. No.: US 2025/0099606 A1
(43) Pub. Date: Mar. 27, 2025

(54) COMPOSITIONS AND METHODS FOR CONJUGATING ACTIVATABLE ANTIBODIES

(71) Applicant: CytomX Therapeutics, Inc., South San Francisco, CA (US)

(72) Inventors: Henry Bernard LOWMAN, El Granada, CA (US); Luc Roland DESNOYERS, South San Francisco, CA (US); Tony W. LIANG, San Mateo, CA (US); Andrei William KONRADI, Burlingame, CA (US); Shweta SINGH, Fremont, CA (US)

(73) Assignee: CytomX Therapeutics, Inc., South San Francisco, CA (US)

(21) Appl. No.: 18/648,088

(22) Filed: Apr. 26, 2024

Related U.S. Application Data
(63) Continuation of application No. 17/227,004, filed on Apr. 9, 2021, now Pat. No. 11,992,528.
Application No. 17/227,004 is a continuation of application No. 15/359,100, filed on Nov. 22, 2016, now abandoned.
Application No. 15/359,100 is a division of application No. 14/296,207, filed on Jun. 4, 2014, now Pat. No. 9,517,276.
(60) Provisional application No. 61/830,913, filed on Jun. 4, 2013.
(60) Provisional application No. 61/919,935, filed on Dec. 23, 2013.

(51) Int. Cl. A61K 47/68; A61K 39/00; A61K 39/395; A61K 49/00; C07K 16/18; C07K 16/28

(52) U.S. Cl. CPC A61K 47/6889; A61K 39/3955; A61K 47/68031; A61K 47/6851; A61K 49/00; C07K 16/18; A61K 2039/505; C07K 16/2863; C07K 2317/21; C07K 2317/76; C07K 2319/00

(57) ABSTRACT

The invention relates generally to compositions and methods for conjugating antibodies and activatable antibodies, and methods of partially reducing antibodies and/or activatable antibodies prior to conjugation, e.g., thiol-based conjugation, with an agent, e.g., a therapeutic and/or diagnostic agent.

### CROSS-REFERENCE TO RELATED APPLICATIONS

This application is a continuation application of U.S. patent application Ser. No. 17/227,004 filed Apr. 9, 2021, which is a continuation application of U.S. patent application Ser. No. 15/359,100 filed Nov. 22, 2016, which is a divisional of U.S. patent application Ser. No. 14/296,207, filed Jun. 4, 2014, no U.S. Pat. No. 9,517,276 issued Dec. 13, 2016, which claims the benefit of U.S. Provisional Application No. 61/830,913, filed Jun. 4, 2013 and U.S. Provisional Application No. 61/919,935, filed Dec. 23, 2013. The contents of each of which are hereby incorporated by reference in their entirety.

### INCORPORATION OF SEQUENCE LISTING

The instant application contains a Sequence Listing which has been submitted electronically in XML format and is hereby incorporated by reference in its entirety. Said XML copy, created on Apr. 22, 2024, is named “4862-120US6-ST26.xml” and is 420,277 bytes in size.

### FIELD OF THE INVENTION

The invention relates generally to compositions and methods for conjugating antibodies and activatable antibodies, and methods of partially reducing antibodies and/or activatable antibodies prior to conjugation, e.g., thiol-based conjugation, with an agent, e.g., a therapeutic and/or diagnostic agent.

### BACKGROUND OF THE INVENTION

Antibody-based therapies have proven effective treatments for some diseases but in some cases, toxicities due to broad target expression have limited their therapeutic effectiveness. In addition, antibody-based therapeutics have exhibited other limitations such as rapid clearance from the circulation following administration. Conjugating agents to antibodies has been used to further advance the use of antibody-based therapies. Molecules such as toxins, radionuclides and drugs including anti-cancer drugs have been conjugated to certain antibodies to generate immunotoxins, radioimmunoconjugates, and/or antibody-drug conjugates (ADCs).

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

Accordingly, there is a continued need in the field of antibody-based therapeutics for antibodies that mimic the desirable characteristics of the small molecule prodrug, as well as a need for improved methods of conjugating agents to these antibodies without negatively impacting their ability to mimic the desirable characteristics of the small molecule prodrug.

### SUMMARY OF THE INVENTION

The present invention provides conjugates that include an activatable antibody and methods of making these activatable antibody conjugates. Also provided are activatable antibodies having points of conjugation for receiving a drug or label. The conjugates can be used therapeutically, diagnostically (e.g., in vitro or in vivo), for in vivo imaging, and for any of a variety of other therapeutic, diagnostic and/or prophylactic uses.

Generally, the compositions and methods provided herein include an activatable antibody that includes an antibody or antibody fragment (AB) that specifically binds a target, where the AB is coupled to a masking moiety (MM) that decreases the ability of the AB to bind its target. In some embodiments, the activatable antibody further includes a cleavable moiety (CM) that is a substrate for a protease. The compositions and methods provided herein enable the attachment of one or more agents to one or more cysteine residues in the AB without compromising the activity (e.g., the masking, activating or binding activity) of the activatable antibody. In some embodiments, the compositions and methods provided herein enable the attachment of one or more agents to one or more cysteine residues in the AB without reducing or otherwise disturbing one or more disulfide bonds within the MM. The compositions and methods provided herein produce an activatable antibody that is conjugated to one or more agents, e.g., any of a variety of therapeutic, diagnostic and/or prophylactic agents, and, in some embodiments, without any of the agent(s) being conjugated to the MM of the activatable antibody. The compositions and methods provided herein produce conjugated activatable antibodies in which the MM retains the ability to effectively and efficiently mask the AB of the activatable antibody in an uncleaved state. The compositions and methods provided herein produce conjugated activatable antibodies in which the activatable antibody is still activated, i.e., cleaved, in the presence of a protease that can cleave the CM.

The activatable antibodies have at least one point of conjugation for an agent, but in the methods and compositions provided herein less than all possible points of conjugation are available for conjugation to an agent. In some embodiments, the one or more points of conjugation are sulfur atoms involved in disulfide bonds. In some embodiments, the one or more points of conjugation are sulfur atoms involved in interchain disulfide bonds. In some embodiments, the one or more points of conjugation are sulfur atoms involved in interchain sulfide bonds, but not sulfur atoms involved in intrachain disulfide bonds. In some embodiments, the one or more points of conjugation are sulfur atoms of cysteine or other amino acid residues containing a sulfur atom. Such residues may occur naturally in the antibody structure or may be incorporated into the antibody by site-directed mutagenesis, chemical conversion, or mis-incorporation of non-natural amino acids.

Also provided are methods of preparing a conjugate of an activatable antibody having one or more interchain disulfide bonds in the AB and one or more intrachain disulfide bonds in the MM, and a drug reactive with free thiols is provided. The method generally includes partially reducing interchain disulfide bonds in the activatable antibody with a reducing agent, such as, for example, TCEP; and conjugating the drug reactive with free thiols to the partially reduced activatable antibody. As used herein, the term partial reduction refers to situations where an activatable antibody is contacted with a reducing agent and less than all disulfide bonds, e.g., less than all possible sites of conjugation are reduced. In some embodiments, less than 99%, 98%, 97%, 96%, 95%, 90%, 85%, 80%, 75%, 70%, 65%, 60%, 55%, 50%, 45%, 40%, 35%, 30%, 25%, 20%, 15%, 10% or less than 5% of all possible sites of conjugation are reduced.

In some embodiments, a method of reducing and conjugating an agent, e.g., a drug, to an activatable antibody resulting in selectivity in the placement of the agent is provided. The method generally includes partially reducing the activatable antibody with a reducing agent such that any conjugation sites in the masking moiety or other non-AB portion of the activatable antibody are not reduced, and conjugating the agent to interchain thiols in the AB. The conjugation site(s) are selected so as to allow desired placement of an agent to allow conjugation to occur at a desired site. The reducing agent is, for example, TCEP. The reduction reaction conditions such as, for example, the ratio of reducing agent to activatable antibody, the length of incubation, the temperature during the incubation, the pH of the reducing reaction solution, etc., are determined by identifying the conditions that produce a conjugated activatable antibody in which the MM retains the ability to effectively and efficiently mask the AB of the activatable antibody in an uncleaved state. The ratio of reduction agent to activatable antibody will vary depending on the activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

In some embodiments, a method of reducing interchain disulfide bonds in the AB of an activatable antibody and conjugating an agent, e.g., a thiol-containing agent such as a drug, to the resulting interchain thiols to selectively locate agent(s) on the AB is provided. The method generally includes partially reducing the AB with a reducing agent to form at least two interchain thiols without forming all possible interchain thiols in the activatable antibody; and conjugating the agent to the interchain thiols of the partially reduced AB. For example, the AB of the activatable antibody is partially reduced for about 1 hour at about 37° C. at a desired ratio of reducing agent:activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

The thiol-containing reagent can be, for example, cysteine or N-acetyl cysteine. The reducing agent can be, for example, TCEP. In some embodiments, the reduced activatable antibody can be purified prior to conjugation, using for example, column chromatography, dialysis, or diafiltration. In some embodiments, the reduced antibody is not purified after partial reduction and prior to conjugation.

In some embodiments, the activatable antibody includes an antibody or antigen-binding fragment thereof (AB) that specifically binds a target, wherein the AB is coupled to a masking moiety (MM), such that coupling of the MM to the AB decreases the ability of the antibody or antigen-binding fragment thereof to bind the target. In some embodiments, the MM is coupled to the AB via a cleavable moiety (CM) that includes a substrate for a protease, for example, a protease that is co-localized with the target at a treatment site in a subject. The activatable antibodies provided herein are stable in circulation, activated at intended sites of therapy and/or diagnosis but not in normal, e.g., healthy, tissue, and, when activated, exhibit binding to the target that is at least comparable to the corresponding, unmodified antibody.

In some embodiments, the activatable antibody in the uncleaved state has the structural arrangement from N-terminus to C-terminus as follows: MM-CM-AB or AB-CM-MM.

In some embodiments, the activatable antibody includes an antibody or antigen-binding fragment thereof (AB) that specifically binds the target. In some embodiments, the antibody or immunologically active fragment thereof that binds the target is a monoclonal antibody, domain antibody, single chain, Fab fragment, a F(ab′)₂ fragment, a scFv, a scAb, a dAb, a single domain heavy chain antibody, and a single domain light chain antibody. In some embodiments, such an antibody or immunologically active fragment thereof that binds the target is a mouse, chimeric, humanized or fully human monoclonal antibody. In some embodiments, the antigen binding fragment thereof is a Fab fragment, a F(ab′)₂ fragment, a scFv, or a scAb.

In some embodiments, the antibody or an antigen binding fragment thereof (AB) specifically binds to a target selected from those shown Table 1. In some embodiments, the AB specifically binds to Epidermal Growth Factor Receptor (EGFR). In some embodiments, the AB specifically binds to Jagged 1 and/or Jagged 2. In some embodiments, the AB specifically binds to interleukin 6 receptor (IL-6R).

In some embodiments, the antibody or an antigen binding fragment thereof (AB) is or is derived from an antibody selected from those shown in Table 2.

In some embodiments, the AB has an equilibrium dissociation constant of about 100 nM or less for binding to the target.

In some embodiments, the MM has an equilibrium dissociation constant for binding to the AB that is greater than the equilibrium dissociation constant of the AB to the target.

In some embodiments, the MM has an equilibrium dissociation constant for binding to the AB that is no more than the equilibrium dissociation constant of the AB to the target.

In some embodiments, the MM does not interfere or compete with the AB of the activatable antibody in a cleaved state for binding to the target.

In some embodiments, the MM is a polypeptide of about 2 to 40 amino acids in length, for example, no more than 40 amino acids long.

In some embodiments, the MM polypeptide sequence is different from that of the target, and the MM polypeptide sequence is no more than 50% identical to any natural binding partner of the AB.

In some embodiments, the MM polypeptide sequence is different from that of the target, and the MM polypeptide sequence is no more than 25% identical to any natural binding partner of the AB. In some embodiments, the MM polypeptide sequence is different from that of the target, and the MM polypeptide sequence is no more than 10% identical to any natural binding partner of the AB.

In some embodiments, the coupling of the MM to the AB decreases the ability of the AB to bind the target such that the dissociation constant (K_{d}) of the AB when coupled to the MM towards the target is at least 20 times greater than the K_{d} of the AB when not coupled to the MM towards the target. In some embodiments, the coupling of the MM to the AB decreases the ability of the AB to bind the target such that the dissociation constant (K_{d}) of the AB when coupled to the MM towards the target is at least 40 times greater than the K_{d} of the AB when not coupled to the MM towards the target. In some embodiments, the coupling of the MM to the AB decreases the ability of the AB to bind the target such that the dissociation constant (K_{d}) of the AB when coupled to the MM towards the target is at least 50 times greater than the K_{d} of the AB when not coupled to the MM towards the target. In some embodiments, the coupling of the MM to the AB decreases the ability of the AB to bind the target such that the K_{d} of the AB when coupled to the MM towards the target is at least 100 times greater than the K_{d} of the AB when not coupled to the MM towards the target. In some embodiments, the coupling of the MM to the AB decreases the ability of the AB to bind the target such that the K_{d} of the AB when coupled to the MM towards the target is at least 1000 times greater than the K_{d} of the AB when not coupled to the MM towards the target. In some embodiments, the coupling of the MM to the AB decreases the ability of the AB to bind the target such that the K_{d} of the AB when coupled to the MM towards the target is at least 10,000 times greater than the K_{d} of the AB when not coupled to the MM towards the target.

In some embodiments, in the presence of the target, the MM decreases the ability of the AB to bind the target by at least 90% when the CM is uncleaved, as compared to when the CM is cleaved when assayed in vitro using a target displacement assay such as, for example, the assay described in PCT Publication Nos. WO 2009/025846 and WO 2010/081173.

In some embodiments, the protease is co-localized with the target in a tissue, and the protease cleaves the CM in the activatable antibody when the activatable antibody is exposed to the protease. In some embodiments, the protease is not active or is significantly less active in tissues that do not significantly express the target. In some embodiments, the protease is not active or is significantly less active in healthy, e.g., non-diseased tissues.

In some embodiments, the CM is a polypeptide of up to 15 amino acids in length.

In some embodiments, the CM is a substrate for a protease selected from the group consisting of those shown in Table 3. In some embodiments, the CM is a substrate for a protease selected from the group consisting of uPA (urokinase plasminogen activator), legumain and MT-SP1 (matriptase). In some embodiments, the protease comprises uPA. In some embodiments, the protease comprises legumain. In some embodiments, the protease comprises MT-SP1.

In some embodiments, the CM is a substrate for at least two proteases. In some embodiments, each protease is selected from the group consisting of those shown in Table 3. In some embodiments, the CM is a substrate for at least two proteases, and one of the proteases is selected from the group consisting of uPA, legumain and MT-SP1 and the other protease is selected from the group consisting of those shown in Table 3. In some embodiments, the CM is a substrate for at least two proteases selected from the group consisting of uPA, legumain and MT-SP1.

In some embodiments, the CM is positioned in the activatable antibody such that in the uncleaved state, binding of the activatable antibody to the target is decreased such that it occurs with an equilibrium dissociation constant that is at least 20-fold greater than the equilibrium dissociation constant of an unmodified AB binding to the target, and whereas the AB of the activatable antibody in the cleaved state binds the target.

In some embodiments, the CM is positioned in the activatable antibody such that in the uncleaved state, binding of the activatable antibody to the target is decreased such that it occurs with an equilibrium dissociation constant that is at least 40-fold greater than the equilibrium dissociation constant of an unmodified AB binding to the target, and whereas the AB of the activatable antibody in the cleaved state binds the target.

In some embodiments, the CM is positioned in the activatable antibody such that in the uncleaved state, binding of the activatable antibody to the target is decreased such that it occurs with an equilibrium dissociation constant that is at least 50-fold greater than the equilibrium dissociation constant of an unmodified AB binding to the target, and whereas the AB of the activatable antibody in the cleaved state binds the target.

In some embodiments, the CM is positioned in the activatable antibody such that in the uncleaved state, binding of the activatable antibody to the target is decreased such that it occurs with an equilibrium dissociation constant that is at least 100-fold greater than the equilibrium dissociation constant of an unmodified AB binding to the target, and whereas the AB of the activatable antibody in the cleaved state binds the target.

In some embodiments, the CM is positioned in the activatable antibody such that in the uncleaved state, binding of the activatable antibody to the target is decreased such that it occurs with an equilibrium dissociation constant that is at least 200-fold greater than the equilibrium dissociation constant of an unmodified AB binding to the target, and whereas the AB of the activatable antibody in the cleaved state binds the target.

In some embodiments, the activatable antibody includes a linking peptide between the MM and the CM.

In some embodiments, the activatable antibody includes a linking peptide between the CM and the AB.

In some embodiments, the activatable antibody includes a first linking peptide (LP1) and a second linking peptide (LP2), and the activatable antibody in the uncleaved state has the structural arrangement from N-terminus to C-terminus as follows: MM-LP1-CM-LP2-AB or AB-LP2-CM-LP1-MM. In some embodiments, the two linking peptides need not be identical to each other.

In some embodiments, each of LP1 and LP2 is a peptide of about 1 to 20 amino acids in length.

In some embodiments, at least one of LP1 or LP2 includes an amino acid sequence selected from the group consisting of (GS)ₙ, (GGS)ₙ, (GSGGS), (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28).

In some embodiments, the activatable antibody is exposed to and cleaved by a protease such that, in the activated or cleaved state, the activated antibody includes a light chain amino acid sequence that includes at least a portion of LP2 and/or CM sequence after the protease has cleaved the CM.

In some embodiments, the activatable antibody also includes a signal peptide. In some embodiments, the signal peptide is conjugated to the activatable antibody via a spacer. In some embodiments, the spacer is conjugated to the activatable antibody in the absence of a signal peptide. In some embodiments, the spacer is joined directly to the MM of the activatable antibody.

In some embodiments, the activatable antibody in an uncleaved state comprises a spacer that is joined directly to the MM and has the structural arrangement from N-terminus to C-terminus of spacer-MM-CM-AB. In some embodiments, the spacer includes at least the amino acid sequence QGQSGQ (SEQ ID NO:11).

In some embodiments, the AB of the activatable antibody naturally contains one or more disulfide bonds. In some embodiments, the AB can be engineered to include one or more disulfide bonds.

In some embodiments, the agent conjugated to the activatable antibody is a therapeutic agent. In some embodiments, the agent conjugated to the activatable antibody is a diagnostic agent. In some embodiments, the agent conjugated to the activatable antibody is a prophylactic agent.

In some embodiments, the agent is an antineoplastic agent. In some embodiments, the agent is a toxin or fragment thereof. As used herein, a fragment of a toxin is a fragment that retains toxic activity. In some embodiments, the agent is an agent selected from the group listed in Table 4. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof.

In some embodiments, the agent is conjugated to the AB via a linker. In some embodiments, the linker is a thiol-containing linker. In some embodiments, the linker is a cleavable linker. In some embodiments, the linker is selected from the group consisting of the linkers shown in Tables 5 and 6.

In some embodiments, the activatable antibody also includes a detectable moiety. In some embodiments, the detectable moiety is a diagnostic agent. In some embodiments, the detectable moiety is a conjugatable detection reagent. In some embodiments, the detectable moiety is, for example, a fluorophore, for example, a fluorescein derivative such as fluorescein isothiocyanate (FITC).

In some embodiments, the activatable antibody and/or conjugated activatable antibody is monospecific. In some embodiments, the activatable antibody and/or conjugated activatable antibody is multispecific, e.g., by way of non-limiting example, bispecific or trifunctional. In some embodiments, the activatable antibody and/or conjugated activatable antibody is formulated as part of a pro-Bispecific T Cell Engager (BITE) molecule. In some embodiments, the activatable antibody and/or conjugated activatable antibody is formulated as part of a pro-Chimeric Antigen Receptor (CAR) modified T cell or other engineered receptor.

In some embodiments, the serum half-life of the activatable antibody is longer than that of the corresponding antibody; e.g., the pK of the activatable antibody is longer than that of the corresponding antibody. In some embodiments, the serum half-life of the activatable antibody is similar to that of the corresponding antibody. In some embodiments, the serum half-life of the activatable antibody is at least 15 days when administered to an organism. In some embodiments, the serum half-life of the activatable antibody is at least 12 days when administered to an organism. In some embodiments, the serum half-life of the activatable antibody is at least 11 days when administered to an organism. In some embodiments, the serum half-life of the activatable antibody is at least 10 days when administered to an organism. In some embodiments, the serum half-life of the activatable antibody is at least 9 days when administered to an organism. In some embodiments, the serum half-life of the activatable antibody is at least 8 days when administered to an organism. In some embodiments, the serum half-life of the activatable antibody is at least 7 days when administered to an organism. In some embodiments, the serum half-life of the activatable antibody is at least 6 days when administered to an organism. In some embodiments, the serum half-life of the conjugated activatable antibody is at least 5 days when administered to an organism. In some embodiments, the serum half-life of the conjugated activatable antibody is at least 4 days when administered to an organism. In some embodiments, the serum half-life of the conjugated activatable antibody is at least 3 days when administered to an organism. In some embodiments, the serum half-life of the conjugated activatable antibody is at least 2 days when administered to an organism. In some embodiments, the serum half-life of the conjugated activatable antibody is at least 24 hours when administered to an organism. In some embodiments, the serum half-life of the conjugated activatable antibody is at least 20 hours when administered to an organism. In some embodiments, the serum half-life of the conjugated activatable antibody is at least 18 hours when administered to an organism. In some embodiments, the serum half-life of the conjugated activatable antibody is at least 16 hours when administered to an organism. In some embodiments, the serum half-life of the conjugated activatable antibody is at least 14 hours when administered to an organism. In some embodiments, the serum half-life of the conjugated activatable antibody is at least 12 hours when administered to an organism. In some embodiments, the serum half-life of the conjugated activatable antibody is at least 10 hours when administered to an organism. In some embodiments, the serum half-life of the conjugated activatable antibody is at least 8 hours when administered to an organism. In some embodiments, the serum half-life of the conjugated activatable antibody is at least 6 hours when administered to an organism. In some embodiments, the serum half-life of the conjugated activatable antibody is at least 4 hours when administered to an organism. In some embodiments, the serum half-life of the conjugated activatable antibody is at least 3 hours when administered to an organism.

The invention provides partially reduced activatable antibodies in which at least one interchain disulfide bond in the activatable antibody has been reduced with a reducing agent without disturbing any intrachain disulfide bonds in the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. In some embodiments, one or more intrachain disulfide bond(s) of the activatable antibody is not disturbed by the reducing agent. In some embodiments, one or more intrachain disulfide bond(s) of the MM within the activatable antibody is not disturbed by the reducing agent. In some embodiments, the activatable antibody in the uncleaved state has the structural arrangement from N-terminus to C-terminus as follows: MM-CM-AB or AB-CM-MM. In some embodiments, reducing agent is TCEP.

The invention also provides partially reduced activatable antibodies in which at least one interchain disulfide bond in the activatable antibody has been reduced with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody. In some embodiments, one or more intrachain disulfide bond(s) of the activatable antibody is not disturbed by the reducing agent. In some embodiments, one or more intrachain disulfide bond(s) of the MM within the activatable antibody is not disturbed by the reducing agent. In some embodiments, the activatable antibody in the uncleaved state has the structural arrangement from N-terminus to C-terminus as follows: MM-CM-AB or AB-CM-MM. In some embodiments, reducing agent is TCEP.

In some embodiments, the partially reduced activatable antibody is conjugated to an agent through at least one interchain thiol. In some embodiments, the agent is selected from the group of agents listed in Table 4. In some embodiments, the agent is a toxin or fragment thereof. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or a derivative thereof. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof. In some embodiments, the agent is conjugated to the AB via a linker. In some embodiments, the linker is a cleavable linker. In some embodiments, the agent is a detectable moiety. In some embodiments, the detectable moiety is a diagnostic agent.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS), (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention also provides partially reduced activatable antibodies in which at least one disulfide bond between the activatable antibody and a second molecule has been reduced with a reducing agent without disturbing any intrachain disulfide bonds in the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. In some embodiments, the second molecule is cysteine. In some embodiments, the second molecule is glutathione.

The invention also provides partially reduced activatable antibodies in which at least one disulfide bond between the activatable antibody and a second molecule has been reduced with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody. In some embodiments, the second molecule is cysteine. In some embodiments, the second molecule is glutathione.

In some embodiments, the partial reduction method does not disturb one or more intrachain disulfide bonds of the activatable antibody. In some embodiments, the method does not disturb one or more intrachain disulfide bonds of the MM within the activatable antibody.

In some embodiments, the activatable antibody in the uncleaved state has the structural arrangement from N-terminus to C-terminus as follows: MM-CM-AB or AB-CM-MM. In some embodiments, reducing agent is TCEP.

In some embodiments, the partially reduced activatable antibody is conjugated to an agent through at least one thiol. In some embodiments, the agent is selected from the group of agents listed in Table 4. In some embodiments, the agent is a toxin or fragment thereof. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or a derivative thereof. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof. In some embodiments, the agent is conjugated to the AB via a linker. In some embodiments, the linker is a cleavable linker. In some embodiments, the agent is a detectable moiety. In some embodiments, the detectable moiety is a diagnostic agent.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS)ₙ (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention provides methods of selectively conjugating an agent to an activatable antibody. For example, the invention provides a method of partially reducing and conjugating an agent to an activatable antibody resulting in selectivity in the placement of the agent by partially reducing at least one interchain disulfide bond in the activatable antibody with a reducing agent without disturbing any intrachain disulfide bonds in the activatable antibody, and conjugating the agent to at least one interchain thiol, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease.

The invention also provides a method of partially reducing and conjugating an agent to an activatable antibody resulting in selectivity in the placement of the agent by partially reducing at least one interchain disulfide bond in the activatable antibody with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody.

In some embodiments, the method does not disturb one or more intrachain disulfide bonds of the activatable antibody. In some embodiments, the method does not disturb one or more intrachain disulfide bonds of the MM within the activatable antibody.

In some embodiments, the activatable antibody in the uncleaved state has the structural arrangement from N-terminus to C-terminus as follows: MM-CM-AB or AB-CM-MM. In some embodiments, reducing agent is TCEP. In some embodiments, the agent is selected from the group of agents listed in Table 4. In some embodiments, the agent is a toxin or fragment thereof. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or a derivative thereof. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof. In some embodiments, the agent is conjugated to the AB via a linker. In some embodiments, the linker is a cleavable linker. In some embodiments, the agent is a detectable moiety. In some embodiments, the detectable moiety is a diagnostic agent.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS)ₙ (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention provides a method of partially reducing and conjugating an agent to an activatable antibody resulting in selectivity in the placement of the agent by partially reducing at least one disulfide bond between the activatable antibody and a second molecule with a reducing agent without disturbing any intrachain disulfide bonds in the activatable antibody, and conjugating the agent to at least one thiol, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. In some embodiments, the second molecule is cysteine. In some embodiments, the second molecule is glutathione.

The invention also provides a method of partially reducing and conjugating an agent to an activatable antibody resulting in selectivity in the placement of the agent by partially reducing at least one disulfide bond between the activatable antibody and a second molecule with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody. In some embodiments, the second molecule is cysteine. In some embodiments, the second molecule is glutathione.

In some embodiments, the method does not disturb one or more intrachain disulfide bonds of the activatable antibody. In some embodiments, the method does not disturb one or more intrachain disulfide bonds of the MM within the activatable antibody.

In some embodiments, the activatable antibody in the uncleaved state has the structural arrangement from N-terminus to C-terminus as follows: MM-CM-AB or AB-CM-MM. In some embodiments, reducing agent is TCEP. In some embodiments, the agent is selected from the group of agents listed in Table 4. In some embodiments, the agent is a toxin or fragment thereof. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or a derivative thereof. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof. In some embodiments, the agent is conjugated to the AB via a linker. In some embodiments, the linker is a cleavable linker. In some embodiments, the agent is a detectable moiety. In some embodiments, the detectable moiety is a diagnostic agent.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS)ₙ (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention also provides a method of partially reducing an activatable antibody resulting in selectivity in the placement of one or more potential conjugation sites in the activatable antibody by partially reducing at least one interchain disulfide bond in the activatable antibody with a reducing agent without disturbing any intrachain disulfide bonds in the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease.

The invention also provides a method of partially reducing an activatable antibody resulting in selectivity in the placement of one or more potential conjugation sites in the activatable antibody by partially reducing at least one interchain disulfide bond in the activatable antibody with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody.

In some embodiments, the method does not disturb one or more intrachain disulfide bonds of the activatable antibody. In some embodiments, the method does not disturb one or more intrachain disulfide bonds of the MM within the activatable antibody.

In some embodiments, the activatable antibody in the uncleaved state has the structural arrangement from N-terminus to C-terminus as follows: MM-CM-AB or AB-CM-MM. In some embodiments, reducing agent is TCEP.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS), (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention also provides a method of partially reducing an activatable antibody resulting in selectivity in the placement of one or more potential conjugation sites in the activatable antibody by partially reducing at least one disulfide bond between the activatable antibody and a second molecule with a reducing agent without disturbing any intrachain disulfide bonds in the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. In some embodiments, the second molecule is cysteine. In some embodiments, the second molecule is glutathione.

The invention also provides a method of partially reducing an activatable antibody resulting in selectivity in the placement of one or more potential conjugation sites in the activatable antibody by partially reducing at least one disulfide bond between the activatable antibody and a second molecule with a reducing agent without disturbing or otherwise compromising the activity and/or efficacy of the activatable antibody, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease. The activity and/or efficacy of the activatable antibody is, by way of nonlimiting example, masking activity, activation of the activatable antibody, and/or binding activity of the activated activatable antibody. In some embodiments, the second molecule is cysteine. In some embodiments, the second molecule is glutathione.

In some embodiments, the method does not disturb one or more intrachain disulfide bonds of the activatable antibody. In some embodiments, the method does not disturb one or more intrachain disulfide bonds of the MM within the activatable antibody.

In some embodiments, the activatable antibody in the uncleaved state has the structural arrangement from N-terminus to C-terminus as follows: MM-CM-AB or AB-CM-MM. In some embodiments, reducing agent is TCEP. In some embodiments, the agent is selected from the group of agents listed in Table 4. In some embodiments, the agent is a toxin or fragment thereof. In some embodiments, the agent is a microtubule inhibitor. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or a derivative thereof. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof. In some embodiments, the agent is conjugated to the AB via a linker. In some embodiments, the linker is a cleavable linker. In some embodiments, the agent is a detectable moiety. In some embodiments, the detectable moiety is a diagnostic agent.

In some embodiments, the target is selected from the group of targets listed in Table 1. In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS)ₙ (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention also provides conjugated activatable antibodies that include an activatable antibody linked to monomethyl auristatin D (MMAD) payload, wherein the activatable antibody includes an antibody or an antigen binding fragment thereof (AB) that specifically binds to a target, a masking moiety (MM) that inhibits the binding of the AB of the activatable antibody in an uncleaved state to the target, and a cleavable moiety (CM) coupled to the AB, and the CM is a polypeptide that functions as a substrate for a protease.

In some embodiments, the MMAD-conjugated activatable antibody can be conjugated using any of several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB.

In some embodiments, the MMAD payload is conjugated to the AB via a linker. In some embodiments, the MMAD payload is conjugated to a cysteine in the AB via a linker. In some embodiments, the MMAD payload is conjugated to a lysine in the AB via a linker. In some embodiments, the MMAD payload is conjugated to another residue of the AB via a linker, such as those residues disclosed herein. In some embodiments, the linker is a thiol-containing linker. In some embodiments, the linker is a cleavable linker. In some embodiments, the linker is a non-cleavable linker. In some embodiments, the linker is selected from the group consisting of the linkers shown in Tables 5 and 6. In some embodiments, the activatable antibody and the MMAD payload are linked via a maleimide caproyl-valine-citrulline linker. In some embodiments, the activatable antibody and the MMAD payload are linked via a malcimide PEG-valine-citrulline linker. In some embodiments, the activatable antibody and the MMAD payload are linked via a malcimide caproyl-valine-citrulline-para-aminobenzyloxycarbonyl linker. In some embodiments, the activatable antibody and the MMAD payload are linked via a malcimide PEG-valine-citrulline-para-aminobenzyloxycarbonyl linker. In some embodiments, the MMAD payload is conjugated to the AB using the partial reduction and conjugation technology disclosed herein.

In some embodiments, the target is selected from the group of targets listed in Table 1. In some embodiments, the target is EGFR. In some embodiments, the target is a Jagged protein, e.g., Jagged 1 and/or Jagged 2. In some embodiments, the target is interleukin 6 receptor (IL-6R). In some embodiments, the AB is or is derived from an antibody selected from the group of antibodies listed in Table 2. In some embodiments, the antigen binding fragment thereof is selected from the group consisting of a Fab fragment, a F(ab′)₂ fragment, a scFv, a scAb, a dAb, a single domain heavy chain antibody, and a single domain light chain antibody. In some embodiments, the AB has an equilibrium dissociation constant of about 100 nM or less for binding to the target. In some embodiments, the MM has an equilibrium dissociation constant for binding to the AB that is greater than the equilibrium dissociation constant of the AB to the target. In some embodiments, the MM does not interfere or compete with the AB of the activatable antibody in a cleaved state for binding to the target. In some embodiments, the MM is a polypeptide of no more than 40 amino acids in length. In some embodiments, the MM polypeptide sequence is different from that of the target, and the MM polypeptide sequence is no more than 50% identical to any natural binding partner of the AB. In some embodiments, the MM does not include more than 25% amino acid sequence identity to the target. In some embodiments, the MM does not include more than 10% amino acid sequence identity to the target. In some embodiments, the CM is a polypeptide of up to 15 amino acids in length. In some embodiments, the protease is co-localized with the target in a tissue, and the protease cleaves the CM in the activatable antibody when the activatable antibody is exposed to the protease. In some embodiments, the activatable antibody includes a linking peptide between the MM and the CM. In some embodiments, the activatable antibody includes a linking peptide between the CM and the AB. In some embodiments, the activatable antibody includes a first linking peptide (LP1) and a second linking peptide (LP2), and the activatable antibody in an uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length. In some embodiments, at least one of LP1 or LP2 includes an amino acid sequence selected from the group consisting of (GS)ₙ, (GGS)ₙ, (GSGGS)ₙ (SEQ ID NO: 21) and (GGGS)ₙ (SEQ ID NO: 22), where n is an integer of at least one. In some embodiments, at least one of LP1 or LP2 includes an amino acid sequence selected from the group consisting of GGSG (SEQ ID NO: 23), GGSGG (SEQ ID NO: 24), GSGSG (SEQ ID NO: 25), GSGGG (SEQ ID NO: 26), GGGSG (SEQ ID NO: 27), and GSSSG (SEQ ID NO: 28). In some embodiments, the CM is a substrate for an enzyme selected from the group consisting of those shown in Table 3. In some embodiments, the activatable antibody in an uncleaved state includes a spacer, and the spacer is joined directly to the MM and has the structural arrangement from N-terminus to C-terminus of spacer-MM-CM-AB.

The invention provides methods of treating, preventing and/or delaying the onset or progression of, or alleviating a symptom of an indication, e.g., disease or disorder, associated with expression and/or activity of the target in a subject using a conjugated activatable antibody that in an activated state binds the target, particularly a conjugated activatable antibody that binds and neutralizes or otherwise inhibits at least one biological activity of the target. Suitable conjugated activatable antibodies for use in any of the methods and kits of the invention include any of the conjugated activatable antibodies described herein, including any partially conjugated activatable antibodies and/or partially reduced activatable antibodies described herein.

In some embodiments, the invention provides methods of treating, preventing and/or delaying the onset or progression of, or alleviating a symptom of an indication, e.g., disease or disorder, associated with a detectable level of expression and/or activity of the target in a subject using a conjugated activatable antibody that in an activated state binds the target, particularly a conjugated activatable antibody that binds and neutralizes or otherwise inhibits at least one biological activity of the target. In some embodiments, the conjugated activatable antibody in an activated state binds the target and is internalized. In some embodiments, the detectable level of expression and/or activity of the target is found in at least one intended site of therapy and/or diagnosis. In some embodiments, the detectable level of expression and/or activity of the target is found in normal, e.g., healthy, tissue, and the conjugated activatable antibody is activated at the intended site(s) of therapy and/or diagnosis but not in the normal, e.g., healthy, tissue. The conjugated activatable antibody is activated, for example, by a protease that is co-localized with the target at the intended site(s) of therapy and/or diagnosis. In some embodiments, the detectable level of expression and/or activity of the target is found in at least one intended site of therapy and/or diagnosis and in normal, e.g., healthy, tissue, and the conjugated activatable antibody is activated at the intended site(s) of therapy and/or diagnosis but not in the normal, e.g., healthy, tissue. The conjugated activatable antibody is activated, for example, by a protease that is co-localized with the target at the intended site(s) of therapy and/or diagnosis.

In some embodiments, the indication, e.g., disease or disorder, associated with expression and/or activity of the target is a cancer. In some embodiments, the indication, e.g., disease or disorder, associated with expression and/or activity of the target is an inflammatory disorder and/or an autoimmune disease.

The invention also provides methods of inhibiting angiogenesis in a subject by administering a therapeutically effective amount of a conjugated activatable antibody described herein to a subject in need thereof.

The conjugated activatable antibody can be administered at any stage of the disease. In some embodiments, a conjugated activatable antibody can be administered to a patient suffering cancer of any stage, from early to metastatic. In some embodiments, a conjugated activatable antibody can be administered to a patient suffering from an inflammatory disorder and/or autoimmune disease of any stage, from early onset to an advanced stage. It is to be understood that the terms subject and patient are used interchangeably herein.

The conjugated activatable antibodies are also useful in other therapeutic indications and treatment regimens. For example, the conjugated activatable antibodies of the embodiments provided herein can be used in a treatment regimen that includes neoadjuvant therapy.

In some embodiments, a conjugated activatable antibody is administered in combination with one or more additional agents such as, by way of non-limiting example, a chemotherapeutic agent, such as an alkylating agent, an anti-metabolite, an anti-microtubule agent, a topoisomerase inhibitor, a cytotoxic antibiotic, and any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art. In some embodiments, the conjugated activatable antibody and the additional agent(s) are formulated in a single composition. In some embodiments, the conjugated activatable antibody and the additional agent(s) are administered as two or more separate compositions. In some embodiments, the conjugated activatable antibody and the additional agent(s) are administered simultaneously. In some embodiments, the conjugated activatable antibody and the additional agent(s) are administered sequentially.

In some embodiments, the subject is a mammal. In some embodiments, the subject is a human. In some embodiments, the subject is a non-human mammal, such as a non-human primate, companion animal (e.g., cat, dog, horse), farm animal, work animal, or zoo animal. In some embodiments, the subject is a rodent. In some embodiments, the subject is a human. In some embodiments, the subject is a companion animal. In some embodiments, the subject is an animal in the care of a veterinarian.

The conjugated activatable antibody and therapeutic formulations thereof are administered to a subject suffering from or susceptible to a disease or disorder associated with expression and/or activity of the target. A subject suffering from or susceptible to a disease or disorder associated with expression and/or activity of the target is identified using any of a variety of methods known in the art. For example, subjects suffering from cancer or other neoplastic condition are identified using any of a variety of clinical and/or laboratory tests such as, physical examination and blood, urine and stool analysis to evaluate health status.

Administration of a conjugated activatable antibody to a patient suffering from a disease or disorder associated with target expression and/or activity is considered successful if any of a variety of laboratory or clinical objectives is achieved. For example, administration of a conjugated activatable antibody to a patient suffering from a disease or disorder associated with target expression and/or activity is considered successful if one or more of the symptoms associated with the disease or disorder is alleviated, reduced, inhibited or does not progress to a further, i.e., worse, state. Administration of a conjugated activatable antibody to a patient suffering from a disease or disorder associated with target expression and/or activity is considered successful if the disease enters remission or does not progress to a further, i.e., worse, state.

The invention also provides methods of using conjugated activatable antibodies that bind the target in a variety of diagnostic and/or prophylactic indications, as well as kits for use in these methods. In some embodiments of these methods and/or kits, the conjugated activatable antibody includes a detectable label. In some embodiments of these methods and/or kits, the detectable label includes an imaging agent, a contrasting agent, an enzyme, a fluorescent label, a chromophore, a dye, one or more metal ions, or a ligand-based label. In some embodiments of these methods and/or kits, the imaging agent comprises a radioisotope. In some embodiments of these methods, the radioisotope is indium or technetium. In some embodiments of these methods, the radioisotope is or is derived from iodine. In some embodiments of these methods, the radioisotope is ¹²⁵I or ¹³³I. In some embodiments of these methods and/or kits, the contrasting agent comprises iodine, gadolinium or iron oxide. In some embodiments of these methods and/or kits, the enzyme comprises horseradish peroxidase, alkaline phosphatase, or β-galactosidase. In some embodiments of these methods and/or kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and/or kits, the luminescent label comprises an N-methylacrydium derivative. In some embodiments of these methods and/or kits, the label comprises an Alexa Fluor® label, such as Alex Fluor® 680 or Alexa Fluor® 750. In some embodiments of these methods and/or kits, the ligand-based label comprises biotin, avidin, streptavidin or one or more haptens.

In some embodiments of these methods and/or kits, the subject is a mammal. In some embodiments of these methods and/or kits, the subject is a human. In some embodiments, the subject is a non-human mammal, such as a non-human primate, companion animal (e.g., cat, dog, horse), farm animal, work animal, or zoo animal. In some embodiments, the subject is a rodent. In some embodiments, the subject is a human. In some embodiments, the subject is a companion animal. In some embodiments, the subject is an animal in the care of a veterinarian.

The invention also provides methods of using the conjugated activatable antibodies (i.e., activatable antibody conjugates) in a variety of diagnostic and/or prophylactic indications. For example, the invention provides methods of detecting presence or absence of a cleaving agent and a target of interest in a subject or a sample by (i) contacting a subject or sample with a conjugated activatable antibody and (ii) measuring a level of conjugated activatable antibody in the subject or sample, wherein a detectable level of activated conjugated activatable antibody in the subject or sample indicates that the cleaving agent and the target are present in the subject or sample and wherein no detectable level of activated conjugated activatable antibody in the subject or sample indicates that the cleaving agent, the target or both the cleaving agent and the target are absent in the subject or sample. In some embodiments an unconjugated activatable antibody corresponding to the activatable antibody conjugated in the conjugated activatable antibody is used to contact the subject or sample.

The invention also provides methods of detecting presence or absence of a cleaving agent in a subject or a sample by (i) contacting a subject or sample with a conjugated activatable antibody in the presence of the target, and (ii) measuring a level of activated conjugated activatable antibody in the subject or sample, wherein a detectable level of activated conjugated activatable antibody in the subject or sample indicates that the cleaving agent is present in the subject or sample and wherein no detectable level of conjugated activatable antibody in the subject or sample indicates that the cleaving agent is absent in the subject or sample. In some embodiments an unconjugated activatable antibody corresponding to the activatable antibody conjugated in the conjugated activatable antibody is used to contact the subject or sample.

The invention also provides methods of detecting presence or absence of a cleaving agent in a subject or a sample by (i) contacting a subject or sample with a conjugated activatable antibody; and (ii) measuring a level of detectable label in the subject or sample, wherein a detectable level of the detectable label in the subject or sample indicates that the cleaving agent is absent in the subject or sample and wherein no detectable level of the detectable label in the subject or sample indicates that the cleaving agent is present in the subject or sample. In some embodiments an unconjugated activatable antibody corresponding to the activatable antibody conjugated in the conjugated activatable antibody is used to contact the subject or sample.

In some embodiments of these methods, the conjugated activatable antibody or corresponding unconjugated activatable antibody includes a detectable label selected from the group consisting of an imaging agent, a contrasting agent, an enzyme, a fluorescent label, a chromophore, a dye, one or more metal ions, and a ligand-based label. In some embodiments of these methods, the imaging agent comprises a radioisotope. In some embodiments of these methods, the radioisotope is indium or technetium. In some embodiments of these methods, the contrasting agent comprises iodine, gadolinium or iron oxide. In some embodiments of these methods, the enzyme comprises horseradish peroxidase, alkaline phosphatase, or β-galactosidase. In some embodiments of these methods, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods, the luminescent label comprises an N-methylacrydium derivative. In some embodiments of these methods, the label comprises an Alexa Fluor® label, such as Alex Fluor® 680 or Alexa Fluor® 750. In some embodiments of these methods, the ligand-based label comprises biotin, avidin, streptavidin or one or more haptens.

In some embodiments of these methods, the subject is a mammal. In some embodiments of these methods, the subject is a human. In some embodiments, the subject is a non-human mammal, such as a non-human primate, companion animal (e.g., cat, dog, horse), farm animal, work animal, or zoo animal. In some embodiments, the subject is a rodent. In some embodiments, the subject is a human. In some embodiments, the subject is a companion animal. In some embodiments, the subject is an animal in the care of a veterinarian.

In some embodiments of these methods, the method is an in vivo method. In some embodiments of these methods, the method is an in situ method. In some embodiments of these methods, the method is an ex vivo method. In some embodiments of these methods, the method is an in vitro method.

In some embodiments of the methods, the method is used to identify or otherwise refine a patient population suitable for treatment with a conjugated activatable antibody of the disclosure. For example, patients that test positive for both the target and a protease that cleaves the substrate in the cleavable moiety (CM) of the conjugated activatable antibody being tested in these methods are identified as suitable candidates for treatment with such a conjugated activatable antibody comprising such a CM. Likewise, patients that test negative for either or both of the target and the protease that cleaves the substrate in the CM in the conjugated activatable antibody or corresponding unconjugated activatable antibody being tested using these methods might be identified as suitable candidates for another form of therapy. In some embodiments, such patients can be tested with other activatable antibodies and/or conjugated activatable antibodies until a suitable conjugated activatable antibody for treatment is identified (e.g., a conjugated activatable antibody comprising a CM that is cleaved by the patient at the site of disease).

In some embodiments of the methods, the method is used to identify or otherwise refine a patient population suitable for treatment with a conjugated activatable antibody of the disclosure followed by treatment by administering that conjugated activatable antibody to a subject in need thereof. For example, patients that test positive for both the target and a protease that cleaves the substrate in the cleavable moiety (CM) of the conjugated activatable antibody or corresponding unconjugated activatable antibody being tested in these methods are identified as suitable candidates for treatment with such a conjugated activatable antibody comprising such a CM, and the patient is then administered a therapeutically effective amount of the conjugated activatable antibody. Likewise, patients that test negative for either or both of the target and the protease that cleaves the substrate in the CM in the conjugated activatable antibody or corresponding unconjugated activatable antibody being tested using these methods might be identified as suitable candidates for another form of therapy. In some embodiments, such patients can be tested with other activatable antibodies and/or conjugated activatable antibodies until a conjugated activatable antibody for treatment is identified (e.g., a conjugated activatable antibody comprising a CM that is cleaved by the patient at the site of disease). In some embodiments, the patient is then administered a therapeutically effective amount of the conjugated activatable antibody for which the patient tested positive.

The invention also provides conjugated activatable antibodies that in an activated state bind a target, wherein the conjugated activatable antibody includes an antibody or an antigen binding fragment thereof (AB) that specifically binds to the target, wherein the AB is conjugated to monomethyl auristatin D (MMAD); a masking moiety (MM) that inhibits the binding of the AB to the target when the activatable antibody is in an uncleaved state; and a cleavable moiety (CM) coupled to the AB, wherein the CM is a polypeptide that functions as a substrate for a protease.

In some embodiments, the activatable antibody in the uncleaved state has the structural arrangement from N-terminus to C-terminus as follows: MM-CM-AB or AB-CM-MM. In some embodiments, the activatable antibody comprises a linking peptide between the MM and the CM. In some embodiments, the activatable antibody comprises a linking peptide between the CM and the AB. In some embodiments, the activatable antibody comprises a first linking peptide (LP1) and a second linking peptide (LP2), and wherein the activatable antibody in the uncleaved state has the structural arrangement from N-terminus to C-terminus as follows: MM-LP1-CM-LP2-AB or AB-LP2-CM-LP1-MM. In some embodiments, the two linking peptides need not be identical to each other. In some embodiments, each of LP1 and LP2 is a peptide of about 1 to 20 amino acids in length.

In some embodiments, the MM has an equilibrium dissociation constant for binding to the AB which is greater than the equilibrium dissociation constant of the AB to the target. In some embodiments, the MM does not interfere or compete with the AB for binding to the target when the activatable antibody is in a cleaved state. In some embodiments, the MM is a polypeptide of about no more than 40 amino acids in length. In some embodiments, the MM polypeptide sequence is different from that of the target and wherein the MM polypeptide sequence is no more than 50% identical to any natural binding partner of the AB.

In some embodiments, the protease is co-localized with the target in a tissue, and wherein the protease cleaves the CM in the activatable antibody when the activatable antibody is exposed to the protease. In some embodiments, the CM is a polypeptide of up to 15 amino acids in length. In some embodiments, the CM is a substrate for an enzyme selected from the group consisting of those shown in Table 3.

In some embodiments, the antigen binding fragment thereof is selected from the group consisting of a Fab fragment, a F(ab′)₂ fragment, a scFv, a scab, a dAb, a single domain heavy chain antibody, and a single domain light chain antibody. In some embodiments, the target for the AB is selected from the group consisting of the targets listed in Table 1. In some embodiments, the AB is or is derived from an antibody listed in Table 2.

In some embodiments, the MMAD is conjugated to the AB via a linker. In some embodiments, linker is a cleavable linker. In some embodiments, the linker is a non-cleavable linker. In some embodiments, the linker is selected from the group consisting of the linkers shown in Tables 5 and 6. In some embodiments, the activatable antibody and the MMAD payload are linked via a maleimide caproyl-valine-citrulline linker. In some embodiments, the activatable antibody and the MMAD payload are linked via a maleimide PEG-valine-citrulline linker. In some embodiments, the activatable antibody and the MMAD payload are linked via a malcimide caproyl-valine-citrulline-para-aminobenzyloxycarbonyl linker. In some embodiments, the activatable antibody and the MMAD payload are linked via a maleimide PEG-valine-citrulline-para-aminobenzyloxycarbonyl linker. In some embodiments, the MMAD payload is conjugated to the AB using the partial reduction and conjugation technology disclosed herein.

Pharmaceutical compositions according to the invention can include a conjugated antibody of the invention and a carrier. These pharmaceutical compositions can be included in kits, such as, for example, diagnostic kits for use in the methods disclosed herein.

### BRIEF DESCRIPTION OF THE DRAWINGS

FIG. 1 is a graph demonstrating that partial reduction of an activatable anti-EGFR antibody using three different TCEP-to-activatable antibody ratios (i.e., ratios of 1.5:1, of 2:1, and of 4:1), and subsequent thiol conjugation of a fluorescent dye (Alexa 680) to such partially reduced activatable anti-EGFR antibody with a cleavable moiety (3954-1204-c225v5) using the methods provided herein successfully conjugates the dye to the activatable antibody, while maintaining the masking efficiency of the masking moiety of the activatable anti-EGFR antibody. As used in these figures, “(1.5)”, “(2)” and “(4)” signify the ratios of TCEP-to-activatable antibody used in the TCEP reduction step.

FIG. 2 is a graph demonstrating that partial reduction of a masked anti-EGFR antibody with a noncleavable moiety using three different TCEP-to-masked antibody ratios (i.e., ratios of 1.5:1, of 2:1, and of 4:1), and subsequent thiol conjugation of Alexa 680 to such partially reduced masked anti-EGFR antibody with a noncleavable moiety (3954-NSUB-c225v5) using the methods provided herein successfully conjugates the dye to the noncleavable masked antibody, while maintaining the masking efficiency of the masking moiety of the masked anti-EGFR antibody. As used in these figures, “(1.5)”, “(2)” and “(4)” signify the ratios of TCEP-to-masked antibody used in the TCEP reduction step.

FIGS. 3A and 3B are an illustration and a graph demonstrating the non-reduced analysis by LabChip® of TCEP reduction of 3954-1204-c225v5, Alexa 680 thiol-conjugation and activation by the protease uPA. As shown in these figures using two different TCEP-to-activatable antibody ratios (i.e., ratios of 1.5:1 and of 4:1), partial reduction, subsequent thiol conjugation of Alexa 680 to the partially reduced activatable anti-EGFR antibody 3954-1204-c225v5 and activation by uPA does not disturb or otherwise negatively affect the activation and/or masking efficiency of the activatable antibody. As used in these figures, “1.5” and “4” signify the ratios of TCEP-to-activatable antibody used in the TCEP reduction step; and “(U)” signifies that the activatable antibody has been activated, i.e., cleaved, by incubation with uPA.

FIGS. 4A and 4B are an illustration and a graph demonstrating the reduced analysis by LabChip® of TCEP reduction of 3954-1204-c225v5, Alexa 680 thiol-conjugation and activation by the protease uPA. As shown in these figures using two different TCEP-to-activatable antibody ratios (i.e., ratios of 1.5:1 and of 4:1), partial reduction, subsequent thiol conjugation of Alexa 680 to the partially reduced activatable anti-EGFR antibody 3954-1204-c225v5 and activation by uPA does not disturb or otherwise negatively affect the activation and/or masking efficiency of the activatable antibody. As used in these figures, “1.5” and “4” signify the ratios of TCEP-to-activatable antibody used in the TCEP reduction step; “(U)” signifies that the activatable antibody has been activated, i.e., cleaved, by incubation with uPA, and “R” signifies reduced analysis.

FIGS. 5A and 5B are an illustration and a graph demonstrating the non-reduced analysis by LabChip® of TCEP reduction of 3954-NSUB-c225v5, Alexa 680 thiol-conjugation and activation by the protease uPA. As shown in these figures using two different TCEP-to-masked antibody ratios (i.e., ratios of 1.5:1 and of 4:1), partial reduction, subsequent thiol conjugation of Alexa 680 to the partially reduced masked anti-EGFR antibody 3954-NSUB-c225v5 and activation by uPA does not disturb or otherwise negatively affect the activation and/or masking efficiency of the noncleavable masked antibody. As used in these figures, “1.5” and “4” signify the ratios of TCEP-to-masked antibody used in the TCEP reduction step; and “(U)” signifies that the noncleavable masked antibody was not activated, i.e., not cleaved, by incubation with uPA.

FIGS. 6A and 6B are an illustration and a graph demonstrating the reduced analysis by LabChip® of TCEP reduction of 3954-NSUB-c225v5, Alexa 680 thiol-conjugation and activation by the protease uPA. As shown in these figures using two different TCEP-to-masked antibody ratios (i.e., ratios of 1.5:1 and of 4:1), partial reduction, subsequent thiol conjugation of Alexa 680 to the partially reduced masked anti-EGFR antibody 3954-NSUB-c225v5 and activation by uPA does not disturb or otherwise negatively affect the activation and/or masking efficiency of the noncleavable masked antibody. As used in these figures, “1.5” and “4” signify the ratios of TCEP-to-masked antibody used in the TCEP reduction step; “(U)” signifies that the noncleavable masked antibody was not been activated, i.e., not cleaved by incubation with uPA, and “R” signifies reduced analysis.

FIG. 7 is a table and a photograph depicting reduction of the activatable anti-Jagged antibody 5342-1204-4D11 at a ratio of TCEP to activatable antibody equaling 4:1 using a 120-minute reduction time. For the data shown, reduction was followed by conjugation to a fluorescent dye, Alexa 680.

FIG. 8 is a graph depicting varying degrees of Alexa 680 conjugation to an anti-Jagged antibody 4D11 or anti-Jagged activatable antibody 5342-1204-4D11 using thiol conjugatable Alexa 680 as a surrogate for thiol conjugatable toxin. This figure also demonstrates that such conjugation can be effected so as to maintain activation of anti-Jagged activatable antibody by uPA.

FIG. 9A is a graph demonstrating the binding activities of anti-Jagged antibody 4D11, antibody conjugate 4D11-vc-MMAD, activatable antibody conjugate 5342-1204-4D11-vc-MMAD, uPA-activated activatable antibody conjugate 5342-1204-4D11-vc-MMAD, and Synagis to the pancreatic adenocarcinoma cell line BxPC3. Cells were incubated with the respective compositions and then stained with AF-647-labelled anti human IgG.

FIG. 9B is a graph demonstrating cytotoxicity activities of anti-Jagged antibody conjugate 4D11-vc-MMAD, activatable antibody conjugate 5342-1204-4D11-vc-MMAD, uPA-activated activatable antibody conjugate 5342-1204-4D11-vc-MMAD, and rituximab antibody conjugated to linker payload vc-MMAD on BxPC3 cells. Viability was measured using Cell Titer Glo reagent and relative Luminescence units plotted against dose.

FIG. 10A is a graph demonstrating the binding activities of anti-Jagged antibody 4D11, antibody conjugate 4D11-vc-MMAE, activatable antibody 5342-1204-4D11, activatable antibody conjugate 5342-1204-4D11-vc-MMAE, uPA-activated activatable antibody conjugate 5342-1204-4D11-vc-MMAE, and Synagis conjugated to linker payload vc-MMAE to the pancreatic adenocarcinoma cell line BxPC3. Cells were incubated with the respective compositions and then stained with AF-647-labelled anti human IgG.

FIG. 10B is a graph demonstrating cytotoxicity activities of anti-Jagged antibody conjugate 4D11-vc-MMAE, activatable antibody conjugate 5342-1204-4D11-vc-MMAE, uPA-activated activatable antibody conjugate 5342-1204-4D11-vc-MMAE, and Synagis conjugated to linker payload vc-MMAE on BxPC3. Viability was measured using Cell Titer Glo reagent and relative Luminescence units plotted against dose.

### DETAILED DESCRIPTION OF THE INVENTION

The present invention provides conjugates that include an activatable antibody and methods of making these activatable antibody conjugates. Also provided are activatable antibodies having points of conjugation for receiving a drug or label. The conjugates can be used therapeutically, diagnostically (e.g., in vitro or in vivo), for in vivo imaging, and for any of a variety of other therapeutic, diagnostic and/or prophylactic uses.

Generally, the compositions and methods provided herein include an activatable antibody that includes an antibody or antibody fragment (AB) that specifically binds a target, where the AB is coupled to a masking moiety (MM) that decreases the ability of the AB to bind its target. In some embodiments, the activatable antibody further includes a cleavable moiety (CM) that is a substrate for a protease. The compositions and methods provided herein enable the attachment of one or more agents to one or more cysteine residues in the AB without compromising the activity (e.g., the masking, activating or binding activity) of the activatable antibody. In some embodiments, the compositions and methods provided herein enable the attachment of one or more agents to one or more cysteine residues in the AB without reducing or otherwise disturbing one or more intrachain disulfide bonds within the activatable antibody. In some embodiments, the compositions and methods provided herein enable the attachment of one or more agents to one or more cysteine residues in the AB without reducing or otherwise disturbing one or more disulfide bonds within the MM.

The compositions and methods provided herein produce an activatable antibody that is conjugated to one or more agents, e.g., any of a variety of therapeutic, diagnostic and/or prophylactic agents, without any of the agent(s) being conjugated to the MM of the activatable antibody. The compositions and methods provided herein produce conjugated activatable antibodies in which the MM retains the ability to effectively mask the AB of the activatable antibody. In addition, such a conjugated activatable antibody retains the ability to be activated, and the activated AB retains the ability to bind to its target.

In some embodiments, the compositions and methods provided herein do not compromise the masking activity and/or masking efficiency of the MM in the activatable antibody. For example, in situations where the compositions and methods might be found to negatively impact the masking activity and/or masking efficiency of the MM in the activatable antibody, the compositions and methods decrease or otherwise disturb the masking activity and/or masking efficiency of the activatable antibody by no more than 50%, no more than 40%, no more than 30%, no more than 25%, no more than 20%, no more than 15%, no more than 10%, no more than 9%, no more than 8%, no more than 7%, no more than 6%, no more than 5%, no more than 4%, no more than 3%, no more than 2% or no more than 1%, as compared to the level of masking activity and/or masking efficiency of the activatable antibody prior to conjugation or in the absence of any conjugation.

In some embodiments, the compositions and methods provided herein do not compromise the activating activity and/or activating efficiency of the activatable antibody. For example, in situations where the compositions and methods might be found to negatively impact the activating activity and/or activating efficiency, the compositions and methods decrease or otherwise disturb the activating activity and/or activating efficiency of the activatable antibody by no more than 50%, no more than 40%, no more than 30%, no more than 25%, no more than 20%, no more than 15%, no more than 10%, no more than 9%, no more than 8%, no more than 7%, no more than 6%, no more than 5%, no more than 4%, no more than 3%, no more than 2% or no more than 1%, as compared to the level of activating activity and/or activating efficiency of the activatable antibody prior to conjugation or in the absence of any conjugation.

In some embodiments, the compositions and methods provided herein do not compromise the binding activity of the activatable antibody. For example, in situations where the compositions and methods might be found to negatively impact the binding activity, the compositions and methods decrease or otherwise disturb the binding activity of the activatable antibody by no more than 50%, no more than 40%, no more than 30%, no more than 25%, no more than 20%, no more than 15%, no more than 10%, no more than 9%, no more than 8%, no more than 7%, no more than 6%, no more than 5%, no more than 4%, no more than 3%, no more than 2% or no more than 1%, as compared to the level of binding activity of the activatable antibody prior to conjugation or in the absence of any conjugation.

The compositions and methods provided herein determine the combination of reagents and reaction conditions to produce the desired partial reduction followed by conjugation. When reduction and subsequent conjugation is not controlled properly, activatable antibodies will be completely reduced, and the masking efficiency of the activatable antibody is/will be compromised.

The conjugated activatable antibodies include an antibody or antigen-binding fragment thereof (AB) that specifically binds a target, and the AB is coupled to a masking moiety (MM), such that coupling of the MM to the AB decreases the ability of the antibody or antigen-binding fragment thereof to bind the target. In some embodiments, the MM is coupled to the AB via a cleavable moiety (CM) that includes a substrate for a protease, for example, a protease that is co-localized with the target at a treatment site in a subject. Numerous studies have demonstrated the correlation of aberrant protease levels, e.g., uPA, legumain, MT-SP1, matrix metalloproteases (MMPs), in solid tumors. (See e.g., Murthy R V, et al. “Legumain expression in relation to clinicopathologic and biological variables in colorectal cancer.” Clin Cancer Res. 11 (2005): 2293-2299; Nielsen B S, et al. “Urokinase plasminogen activator is localized in stromal cells in ductal breast cancer.” Lab Invest 81 (2001): 1485-1501; Mook O R, et al. “In situ localization of gelatinolytic activity in the extracellular matrix of metastases of colon cancer in rat liver using quenched fluorogenic DQ-gelatin.” J Histochem Cytochem. 51 (2003): 821-829).

The conjugated activatable antibodies provided herein include a substrate for a protease, which is useful in leveraging the protease activity in tumor cells for targeted conjugated antibody activation at the site of treatment and/or diagnosis. The substrate selection process is used to identify substrates that have a number of desirable characteristics. For example, the selected substrates are systemically stable (i.e., stable in the systemic circulation of a subject), are generally not susceptible to cleavage by circulating proteases such as plasmin, thrombin, tissue plasminogen activator (tPA) or a kallikrein (KLK) such as KLK-5 and/or KLK-7, are non-toxic, are generally not susceptible to cleavage at potential sites of toxicity such as the skin by proteases such as ADAM 9, ADAM 10, ADAM 17 and/or kallikreins, such as KLK-5 and KLK-7, and are active at an intended site of treatment and/or diagnosis. In some embodiments, the identified substrates are selected for proteases that are dysregulated, due to, for example, being overexpressed or showing excess activity, or being less susceptible to protease inhibition (due, e.g., to underexpression of the corresponding inhibitor or reduction in inhibitor activity) at an intended site of therapy and/or diagnosis but are not typically expressed at or in normal, healthy or otherwise non-diseased or damaged tissue, and then the selected substrates are subsequently counter-screened against proteases expressed in normal, e.g., non-diseased, tissue.

As a non-limiting example, the AB is a binding partner for any target listed in Table 1.

TABLE 1
Exemplary Targets

| | | | | | |
|---|---|---|---|---|---|
| 1-92-LFA-3 | CD52 | DL44 | ICOS | LAG-3 | TAPA1 |
| Alpha-4 | CD56 | DLL4 | IFNalpha | LIF-R | TGFb eta |
| integrin | | | | | |
| Alpha-V | CD64 | DPP-4 | IFNbeta | LIGHT | TIGIT |
| integrin | | | | | |
| alpha4beta1 | CD70 | EGFR | IFNgamma | MRP4 | TIM-3 |
| integrin | | | | | |
| alpha4beta7 | CD74 | Endothelin B | IgE | MUC1 | TLR2 |
| integrin | | receptor | | | |
| | | (ETBR) | | | |
| AGR2 | CD80 | EpCAM | IgE Receptor | Mucin-16 | TLR4 |
| | | | (FceRI) | | |
| Anti-Lewis-Y | CD81 | EPHA2 | IGF | Na/K | TLR6 |
| | | | | ATPase | |
| Apelin J | CD86 | ERBB3 | IGF1R | Neutrophil | TLR7 |
| receptor | | | | elastase | |
| APRIL | CD95 | F protein of | IL1B | NGF | TLR8 |
| | | RSV | | | |
| B7-H4 | CD117 | FAP | IL1R | Nicastrin | TLR9 |
| BAFF | CD125 | FGF-2 | IL2 | Notch | TMEM31 |
| | | | | Receptors | |
| BTLA | CD132 | FGF8 | IL11 | Notch 1 | TNFalpha |
| | (IL-2RG) | | | | |
| C5 | CD133 | FGFR1 | IL12 | Notch 2 | TNFR |
| complement | | | | | |
| C-242 | CD137 | FGFR2 | IL12p40 | Notch 3 | TNFRS12A |
| CD2 | CD138 | FGFR3 | IL-12R, | Notch 4 | TRAIL-R1 |
| | | | IL-12Rbeta1 | | |
| CD3 | CD166 | FGFR4 | IL13 | NOV | TRAIL-R2 |
| CD6 | CD172A | Folate | IL13R | OSM-R | Transferrin |
| | | receptor | | | |
| CD9 | CD248 | G-CSF | IL15 | OX-40 | Transferrin |
| | | | | | receptor |
| CD11a | CEACAM5 | G-CSFR | IL17 | PAR2 | TRK-A |
| | (CEA) | | | | |
| CD19 | CEACAM6 | GD2 | IL18 | PDGF-AA | TRK-B |
| | (NCA-90) | | | | |
| CD20 | CLAUDIN- | GITR | IL21 | PDGF-BB | uPAR |
| | 3 | | | | |
| CD22 | CLAUDIN- | GLUT1 | IL23 | PDGFRalpha | VAP1 |
| | 4 | | | | |
| CD24 | cMet | GLUT4 | IL23R | PDGFRbeta | VCAM-1 |
| CD25 | Collagen | GM-CSF | IL27/IL27R | PD-1 | VEGF |
| | | | (wsx1) | | |
| CD27 | Cripto | GM-CSFR | IL29 | PD-L1 | VEGF-A |
| CD28 | CSFR | GP Ilb/IIIa | IL-31R | PD-L2 | VEGF-B |
| | | receptors | | | |
| CD30 | CSFR-1 | Gp130 | IL31/IL31R | Phosphatidyl- | VEGF-C |
| | | | | serine | |
| CD33 | CTLA-4 | GPIIB/IIIA | IL2R | P1GF | VEGF-D |
| CD38 | CTGF | GPNMB | IL4 | PSCA | VEGFR1 |
| CD40 | CXCL10 | GRP78 | IL4R | PSMA | VEGFR2 |
| CD40L | CXCL13 | HER2/neu | IL6, IL6R | RAAG12 | VEGFR3 |
| CD41 | CXCR1 | HGF | Insulin | RAGE | VISTA |
| | | | Receptor | | |
| CD44 | CXCR2 | hGH | Jagged | SLC44A4 | WISP-1 |
| | | | Ligands | | |
| CD47 | CXCR4 | HVEM | Jagged 1 | Sphingosine WISP-2 | |
| | | | | 1 Phosphate | |
| CD51 | CYR61 | Hyaluronidase | Jagged 2 | STEAP1 | WISP-3 |

As a nonlimiting example, the AB is or is derived from an antibody listed in Table 2.

TABLE 2
Exemplary sources for Abs

| Antibody Trade Name | |
|---|---|
| (antibody name) | Target |
| Avastin ™ (bevacizumab) | VEGF |
| Lucentis ™ (ranibizumab) | VEGF |
| Erbitux ™ (cetuximab) | EGFR |
| Vectibix ™ (panitumumab) | EGFR |
| Remicade ™ (infliximab) | TNFα |
| Humira ™ (adalimumab) | TNFα |
| Tysabri ™ (natalizumab) | Integrinα4 |
| Simulect ™ (basiliximab) | IL2R |
| Soliris ™ (eculizumab) | Complement C5 |
| Raptiva ™ (efalizumab) | CD11a |
| Bexxar ™ (tositumomab) | CD20 |
| Zevalin ™ (ibritumomab tiuxetan) | CD20 |
| Rituxan ™ (rituximab) | CD20 |
| Ocrelizumab | CD20 |
| Arzerra ™ (ofatumumab) | CD20 |
| Obinutuzumab | CD20 |
| Zenapax ™ (daclizumab) | CD25 |
| Adcetris ™ (brentuximab vedotin) | CD30 |
| Myelotarg ™ (gemtuzumab) | CD33 |
| Mylotarg ™ (gemtuzumab ozogamicin) | CD33 |
| Campath ™ (alemtuzumab) | CD52 |
| ReoPro ™ (abiciximab) | Glycoprotein receptor IIb/IIIa |
| Xolair ™ (omalizumab) | IgE |
| Herceptin ™ (trastuzumab) | Her2 |
| Kadcyla ™ (trastuzumab emtansine) | Her2 |
| Synagis ™ (palivizumab) | F protein of RSV |
| (ipilimumab) | CTLA-4 |
| (tremelimumab) | CTLA-4 |
| Hu5c8 | CD40L |
| (pertuzumab) | Her2-neu |
| (ertumaxomab) | CD3/Her2-neu |
| Orencia ™ (abatacept) | CTLA-4 |
| (tanezumab) | NGF |
| (bavituximab) | Phosphatidylserine |
| (zalutumumab) | EGFR |
| (mapatumumab) | EGFR |
| (matuzumab) | EGFR |
| (nimotuzumab) | EGFR |
| ICR62 | EGFR |
| mAb 528 | EGFR |
| CH806 | EGFR |
| MDX-447 | EGFR/CD64 |
| (edrecolomab) | EpCAM |
| RAV12 | RAAG12 |
| huJ591 | PSMA |
| Enbrel ™ (etanercept) | TNF-R |
| Amevive ™ (alefacept) | 1-92-LFA-3 |
| Antril ™, Kineret ™ (ankinra) | IL-1Ra |
| GC1008 | TGFbeta |
| | Notch, e.g., Notch 1 |
| | Jagged 1 or Jagged 2 |
| (adecatumumab) | EpCAM |
| (figitumumab) | IGF1R |
| (tocilizumab) | IL-6 receptor |
| Stelara ™ (ustekinumab) | IL-12/IL-23 |
| Prolia ™ (denosumab) | RANKL |

In some embodiments, the AB binds Epidermal Growth Factor Receptor (EGFR). In some embodiments, the AB that binds EGFR includes one or more of the heavy chain and/or light chain sequences shown below.

C225v5 Antibody Heavy Chain Nucleotide Sequence:
(SEQ ID NO: 1)
CAGGTGCAGCTGAAACAGAGCGGCCCGGGCCTGGTGCAGCCGAGCCAGA
GCCTGAGCATTACCTGCACCGTGAGCGGCTTTAGCCTGACCAACTATGG
CGTGCATTGGGTGCGCCAGAGCCCGGGCAAAGGCCTGGAATGGCTGGGC
GTGATTTGGAGCGGCGGCAACACCGATTATAACACCCCGTTTACCAGCC
GCCTGAGCATTAACAAAGATAACAGCAAAAGCCAGGTGTTTTTTAAAAT
GAACAGCCTGCAAAGCCAGGATACCGCGATITATTATTGCGCGCGCGCG
CTGACCTATTATGATTATGAATTIGCGTATTGGGGCCAGGGCACCCTGG
TGACCGTGAGCGCGGCTAGCACCAAGGGCCCATCGGTCTTCCCCCTGGC
ACCCTCCTCCAAGAGCACCTCTGGGGGCACAGCGGCCCTGGGCTGCCTG
GTCAAGGACTACTTCCCCGAACCGGTGACGGTGTCGTGGAACTCAGGCG
CCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGC
ACCCAGACCTACATCTGCAACGTGAATCACAAGCCCAGCAACACCAAGG
TGGACAAGAAAGTTGAGCCCAAATCTTGTGACAAAACTCACACATGCCC
ACCGTGCCCAGCACCTGAACTCCTGGGGGGACCGTCAGTCTTCCTCTTC
CCCCCAAAACCCAAGGACACCCTCATGATCTCCCGGACCCCTGAGGTCA
CATGCGTGGTGGTGGACGTGAGCCACGAAGACCCTGAGGTCAAGTTCAA
CTGGTACGTGGACGGCGTGGAGGTGCATAATGCCAAGACAAAGCCGCGG
GAGGAGCAGTACAACAGCACGTACCGTGTGGTCAGCGTCCTCACCGTCC
TGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGG
CAGCCCCGAGAACCACAGGTGTACACCCTGCCCCCATCCCGGGATGAAC
TGACCAAGAACCAGGTCAGCCTGACCTGCCTGGTCAAAGGCTTCTATCC
CAGCGACATCGCCGTGGAGTGGGAGAGCAATGGGCAGCCGGAGAACAAC
TACAAGACCACGCCTCCCGTGCTGGACTCCGACGGCTCCTTCTTCCTCT
ACAGCAAGCTCACCGTGGACAAGAGCAGGTGGCAGCAGGGGAACGTCTT
CTCATGCTCCGTGATGCATGAGGCTCTGCACAACCACTACACGCAGAAG
AGCCTCTCCCTGTCTCCGGGTAAATGA
C225v5 Antibody Heavy Chain Amino Acid Sequence
(SEQ ID NO: 2)
QVQLKQSGPGLVQPSQSLSITCTVSGFSLTNYGVHWVRQSPGKGLEWLG
VIWSGGNTDYNTPFTSRLSINKDNSKSQVFFKMNSLQSQDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK*
C225v5 Antibody Light Chain Nucleotide Sequence:
(SEQ ID NO: 10)
CAGATCTTGCTGACCCAGAGCCCGGTGATTCTGAGCGTGAGCCCGGGCG
AACGTGTGAGCTTTAGCTGCCGCGCGAGCCAGAGCATTGGCACCAACAT
TCATTGGTATCAGCAGCGCACCAACGGCAGCCCGCGCCTGCTGATTAAA
TATGCGAGCGAAAGCATTAGCGGCATTCCGAGCCGCTTTAGCGGCAGCG
GCAGCGGCACCGATTTTACCCTGAGCATTAACAGCGTGGAAAGCGAAGA
TATTGCGGATTATTATTGCCAGCAGAACAACAACTGGCCGACCACCTTT
GGCGCGGGCACCAAACTGGAACTGAAACGTACGGTGGCTGCACCATCTG
TCTTCATCTTCCCGCCATCTGATGAGCAGTTGAAATCTGGAACTGCCTC
TGTTGTGTGCCTGCTGAATAACTTCTATCCCAGAGAGGCCAAAGTACAG
TGGAAGGTGGATAACGCCCTCCAATCGGGTAACTCCCAGGAGAGTGTCA
CAGAGCAGGACAGCAAGGACAGCACCTACAGCCTCAGCAGCACCCTGAC
GCTGAGCAAAGCAGACTACGAGAAACACAAAGTCTACGCCTGCGAAGTC
ACCCATCAGGGCCTGAGCTCGCCCGTCACAAAGAGCTTCAACAGGGGAG
AGTGTTAG
C225v5 Antibody Light Chain Amino Acid Sequence:
(SEQ ID NO: 16)
QILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC*
C225v4 Antibody Heavy Chain Nucleotide Sequence:
(SEQ ID NO: 238)
CAGGTGCAGCTGAAACAGAGCGGCCCGGGCCTGGTGCAGCCGAGCCAGA
GCCTGAGCATTACCTGCACCGTGAGCGGCTTTAGCCTGACCAACTATGG
CGTGCATTGGGTGCGCCAGAGCCCGGGCAAAGGCCTGGAATGGCTGGGC
GTGATTTGGAGCGGCGGCAACACCGATTATAACACCCCGTTTACCAGCC
GCCTGAGCATTAACAAAGATAACAGCAAAAGCCAGGTGTTTTTTAAAAT
GAACAGCCTGCAAAGCAACGATACCGCGATTTATTATTGCGCGCGCGCG
CTGACCTATTATGATTATGAATTTGCGTATTGGGGCCAGGGCACCCTGG
TGACCGTGAGCGCGGCTAGCACCAAGGGCCCATCGGTCTTCCCCCTGGC
ACCCTCCTCCAAGAGCACCTCTGGGGGCACAGCGGCCCTGGGCTGCCTG
GTCAAGGACTACTTCCCCGAACCGGTGACGGTGTCGTGGAACTCAGGCG
CCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGC
ACCCAGACCTACATCTGCAACGTGAATCACAAGCCCAGCAACACCAAGG
TGGACAAGAAAGTTGAGCCCAAATCTTGTGACAAAACTCACACATGCCC
ACCGTGCCCAGCACCTGAACTCCTGGGGGGACCGTCAGTCTTCCTCTTC
CCCCCAAAACCCAAGGACACCCTCATGATCTCCCGGACCCCTGAGGTCA
CATGCGTGGTGGTGGACGTGAGCCACGAAGACCCTGAGGTCAAGTTCAA
CTGGTACGTGGACGGCGTGGAGGTGCATAATGCCAAGACAAAGCCGCGG
GAGGAGCAGTACAACAGCACGTACCGTGTGGTCAGCGTCCTCACCGTCC
TGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGG
CAGCCCCGAGAACCACAGGTGTACACCCTGCCCCCATCCCGGGATGAAC
TGACCAAGAACCAGGTCAGCCTGACCTGCCTGGTCAAAGGCTTCTATCC
CAGCGACATCGCCGTGGAGTGGGAGAGCAATGGGCAGCCGGAGAACAAC
TACAAGACCACGCCTCCCGTGCTGGACTCCGACGGCTCCTTCTTCCTCT
ACAGCAAGCTCACCGTGGACAAGAGCAGGTGGCAGCAGGGGAACGTCTT
CTCATGCTCCGTGATGCATGAGGCTCTGCACAACCACTACACGCAGAAG
AGCCTCTCCCTGTCTCCGGGTAAATGA
C225v4 Antibody Heavy Chain Amino Acid Sequence:
(SEQ ID NO: 239)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK*
C225v6 Antibody Heavy Chain Nucleotide Sequence:
(SEQ ID NO: 240)
CAGGTGCAGCTGAAACAGAGCGGCCCGGGCCTGGTGCAGCCGAGCCAGA
GCCTGAGCATTACCTGCACCGTGAGCGGCTTTAGCCTGACCAACTATGG
CGTGCATTGGGTGCGCCAGAGCCCGGGCAAAGGCCTGGAATGGCTGGGC
GTGATTTGGAGCGGCGGCAACACCGATTATAACACCCCGTTTACCAGCC
GCCTGAGCATTAACAAAGATAACAGCAAAAGCCAGGTGTTTTTTAAAAT
GAACAGCCTGCAAAGCCAGGATACCGCGATTTATTATTGCGCGCGCGCG
CTGACCTATTATGATTATGAATTTGCGTATTGGGGCCAGGGCACCCTGG
TGACCGTGAGCGCGGCTAGCACCAAGGGCCCATCGGTCTTCCCCCTGGC
ACCCTCCTCCAAGAGCACCTCTGGGGGCACAGCGGCCCTGGGCTGCCTG
GTCAAGGACTACTTCCCCGAACCGGTGACGGTGTCGTGGAACTCAGGCG
CCCTGACCAGCGGCGTGCACACCTTCCCGGCTGTCCTACAGTCCTCAGG
ACTCTACTCCCTCAGCAGCGTGGTGACCGTGCCCTCCAGCAGCTTGGGC
ACCCAGACCTACATCTGCAACGTGAATCACAAGCCCAGCAACACCAAGG
TGGACAAGAAAGTTGAGCCCAAATCTTGTGACAAAACTCACACATGCCC
ACCGTGCCCAGCACCTGAACTCCTGGGGGGACCGTCAGTCTTCCTCTTC
CCCCCAAAACCCAAGGACACCCTCATGATCTCCCGGACCCCTGAGGTCA
CATGCGTGGTGGTGGACGTGAGCCACGAAGACCCTGAGGTCAAGTTCAA
CTGGTACGTGGACGGCGTGGAGGTGCATAATGCCAAGACAAAGCCGCGG
GAGGAGCAGTACGCCAGCACGTACCGTGTGGTCAGCGTCCTCACCGTCC
TGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGG
CAGCCCCGAGAACCACAGGTGTACACCCTGCCCCCATCCCGGGATGAAC
TGACCAAGAACCAGGTCAGCCTGACCTGCCTGGTCAAAGGCTTCTATCC
CAGCGACATCGCCGTGGAGTGGGAGAGCAATGGGCAGCCGGAGAACAAC
TACAAGACCACGCCTCCCGTGCTGGACTCCGACGGCTCCTTCTTCCTCT
ACAGCAAGCTCACCGTGGACAAGAGCAGGTGGCAGCAGGGGAACGTCTT
CTCATGCTCCGTGATGCATGAGGCTCTGCACAACCACTACACGCAGAAG
AGCCTCTCCCTGTCTCCGGGTAAATGA]
C225v6 Antibody Heavy Chain Amino Acid Sequence
(SEQ ID NO: 241)
QVQLKQSGPGLVQPSQSLSITCTVSGFSLTNYGVHWVRQSPGKGLEWLG
VIWSGGNTDYNTPFTSRLSINKDNSKSQVFFKMNSLQSQDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYASTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK*

In some embodiments, the AB binds interleukin 6 receptor (IL-6R). In some embodiments, the AB that binds IL-6R includes one or more of the heavy chain and/or light chain sequences shown below.

Av1 Antibody Heavy Chain Amino Acid Sequence:
(SEQ ID NO: 242)
QVQLQESGPGLVRPSQTLSLTCTVSGYSITSDHAWSWVRQPPGRGLEWIG
YISYSGITTYNPSLKSRVTISRDNSKNTLYLQMNSLRAEDTAVYYCARSL
ARTTAMDYWGQGSLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTIPPVL
DSDGSFFLYSKLTVDKSRWQQGNVFSCSVMHEALHNHYTQKSLSLSPGK
Av1 Antibody Light Chain Amino Acid Sequence:
(SEQ ID NO: 243)
DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, the AB binds a Jagged target, e.g., Jagged 1, Jagged 2 or both Jagged 1 and Jagged 2. In some embodiments, the AB that binds a Jagged target includes one or more of the heavy chain and/or light chain sequences shown below.

4D11 Light Chain sequence:
(SEQ ID NO: 244)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
4D11 Heavy Chain sequence:
(SEQ ID NO: 245)
EVQLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
4D11v2 Heavy Chain sequence
(SEQ ID NO: 246)
EVHLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
4D11v2 Light Chain Sequence
(SEQ ID NO: 247)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKRTVAAPSVFIFPPSDEQLKSGTASVVCLLNNFYPREAKVQWKV
DNALQSGNSQESVTEQDSKDSTYSLSSTLTLXKADYEKHKVYACEVTHQG
LSSPVTKSFNRGEC

In some embodiments, the AB that binds a Jagged target includes one or more of the variable heavy chain and/or variable light chain sequences shown below.

Variable Light Chain Amino Sequence Lc4
(SEQ ID NO: 248)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc4
(SEQ ID NO: 249)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKDI
GGRSAFDYWGQGTLVTVSS
Variable Light Chain Amino Sequence Lc5
(SEQ ID NO: 250)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc5
(SEQ ID NO: 251)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PYHGQFDYWGQGTLVTVSS
Variable Light Chain Amino Sequence Lc7
(SEQ ID NO: 252)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc7
(SEQ ID NO: 253)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PFFGQFDYWGQGTLVTVSS
Variable Light Chain Amino Sequence Lc8
(SEQ ID NO: 254)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc8
(SEQ ID NO: 255)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKHI
GRTNPFDYWGQGTLVTVSS
Variable Light Chain Amino Sequence Lc13
(SEQ ID NO: 256)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc13
(SEQ ID NO: 257)
EVQLLESGGGLVQPGGSLRLSCAASGFTFSSYAMSWVRQAPGKGLEWVSS
IEQMGWQTEYADSVKGRFTISRDNSKNTLYLQMNSLRAEDTAVYYCAKSA
AAFDYWGQGTLVTVSS
Variable Light Chain Amino Sequence Lc16
(SEQ ID NO: 258)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc16
(SEQ ID NO: 259)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PYYGQFDYWGQGTLVTVSS
Variable Light Chain Amino Sequence Lc19
(SEQ ID NO: 260)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc19
(SEQ ID NO: 261)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PFFGQFDYWGQGTLVTVSS
Variable Light Chain Amino Sequence Lc21
(SEQ ID NO: 262)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc21
(SEQ ID NO: 263)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKDI
GGRSAFDYWGQGTLVTVSS
Variable Light Chain Amino Sequence Lc24
(SEQ ID NO: 264)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc24
(SEQ ID NO: 265)
EVQLLESGGGLVQPGGSLRLSCAASGFTFSSYAMSWVRQAPGKGLEWVSS
IEEMGWQTLYADSVKGRFTISRDNSKNTLYLQMNSLRAEDTAVYYCAKSA
AAFDYWGQGTLVTVSS
Variable Light Chain Amino Sequence Lc26
(SEQ ID NO: 266)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc26
(SEQ ID NO: 267)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKDI
GGRSAFDYWGQGTLVTVSS
Variable Light Chain Amino Sequence Lc27
(SEQ ID NO: 268)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc27
(SEQ ID NO: 269)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PFYGQFDYWGQGTLVTVSS
Variable Light Chain Amino Sequence Lc28
(SEQ ID NO: 270)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc28
(SEQ ID NO: 271)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PFFGQFDYWGQGTLVTVSS
Variable Light Chain Amino Sequence Lc30
(SEQ ID NO: 272)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc30
(SEQ ID NO: 273)
EVQLLESGGGLVQPGGSLRLSCAASGFTFSSYAMSWVRQAPGKGLEWVSS
IEEMGWQTLYADSVKGRFTISRDNSKNTLYLQMNSLRAEDTAVYYAKSAA
AFDYWGQGTLVTVSS
Variable Light Chain Amino Sequence Lc31
(SEQ ID NO: 274)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc31
(SEQ ID NO: 275)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKDI
GGRSAFDYWGQGTLVTVSS
Variable Light Chain Amino Sequence Lc32
(SEQ ID NO: 276)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc32
(SEQ ID NO: 277)
EVQLLESGGGLVQPGGSLRLSCAASGFTFSSYAMSWVRQAPGKGLEWVSS
IDPEGWQTYYADSVKGRFTISRDNSKNTLYLQMNSLRAEDTAVYYCAKSA
AAFDYWGQGTLVTVSS
Variable Light Chain Amino Sequence Lc37
(SEQ ID NO: 278)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc37
(SEQ ID NO: 279)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PHNGQFDYWGQGTLVTVSS
Variable Light Chain Amino Sequence Lc39
(SEQ ID NO: 280)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc39
(SEQ ID NO: 281)
EVQLLESGGGLVQPGGSLRLSCAASGFTFSSYAMSWVRQAPGKGLEWVSS
IEQMGWQTEYADSVKGRFTISRDNSKNTLYLQMNSLRAEDTAVYYCAKSA
AAFDYWGQGTLVTVSS
Variable Light Chain Amino Sequence Lc40
(SEQ ID NO: 282)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Heavy Chain Amino Sequence Hc40
(SEQ ID NO: 283)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKSP
PFFGQFDYWGQGTLVTVSS
Variable Light Chain Amino Sequence Lc47
(SEQ ID NO: 284)
DIQMTQSPSSLSASVGDRVTITCRASQSISSYLNWYQQKPGKAPKLLIYA
ASSLQSGVPSRFSGSGSGTDFTLTISSLQPEDFATYYCQQSVVAPLTFGQ
GTKVEIKR
Variable Heavy Chain Amino Sequence Hc47
(SEQ ID NO: 285)
EVQLLESGGGLVQPGGSLRLSCAASGFTFSSYAMSWVRQAPGKGLEWVSS
IDEMGWQTEYADSVKGRFTISRDNSKNTLYLQMNSLRAEDTAVYYCAKSA
AAFDYWGQGTLVTVSS
Variable 4B2 Light Chain
(SEQ ID NO: 286)
DIQMTQSPSSLSASVGDRVTITCRASQSISSYLNWYQQKPGKAPKLLIYA
ASSLQSGVPSRFSGSGSGTDFTLTISSLQPEDFATYYCQQTLDAPPQFGQ
GTKVEIKR
Variable 4B2 Heavy Chain
(SEQ ID NO: 287)
EVQLLESGGGLVQPGGSLRLSCAASGFTFSSYAMSWVRQAPGKGLEWVSS
IEQMGWQTYYADSVKGRFTISRDNSKNTLYLQMNSLRAEDTAVYYCAKDI
GGRSAFDYWGQGTLVTVSS
Variable 4D11 Light Chain
(SEQ ID NO: 288)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKR
Variable 4D11 Heavy Chain
(SEQ ID NO: 289)
EVQLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSS
Variable 4E7 Light Chain
(SEQ ID NO: 290)
DIQMTQSPSSLSASVGDRVTITCRASQSISSYLNWYQQKPGKAPKLLIYA
ASSLQSGVPSRFSGSGSGTDFTLTISSLQPEDFATYYCQQSLVAPLTFGQ
GTKVEIKR
Variable 4E7 Heavy Chain
(SEQ ID NO: 291)
EVQLLESGGGLVQPGGSLRLSCAASGFTFSSYAMSWVRQAPGKGLEWVSS
IEEMGWQTKYADSVKGRFTISRDNSKNTLYLQMNSLRAEDTAVYYCAKSA
AAFDYWGQGTLVTVSS
Variable 4E11 Light Chain
(SEQ ID NO: 292)
DIQMTQSPSSLSASVGDRVTITCRASQSISSYLNWYQQKPGKAPKLLIYA
ASSLQSGVPSRFSGSGSGTDFTLTISSLQPEDFATYYCQQALDAPLMFGQ
GTKVEIKR
Variable 4E11 Heavy Chain
(SEQ ID NO: 293)
EVQLLESGGGLVQPGGSLRLSCAASGFTFSSYAMSWVRQAPGKGLEWVSS
IEPMGQLTEYADSVKGRFTISRDNSKNTLYLQMNSLRAEDTAVYYCAKDI
GGRSAFDYWGQGTLVTVSS
Variable 6B7 Light Chain
(SEQ ID NO: 294)
DIQMTQSPSSLSASVGDRVTITCRASQSISSYLNWYQQKPGKAPKLLIYA
ASSLQSGVPSRFSGSGSGTDFTLTISSLQPEDFATYYCQQALVAPLTFGQ
GTKVEIKR
Variable 6B7 Heavy Chain
(SEQ ID NO: 295)
EVQLLESGGGLVQPGGSLRLSCAASGFTFSSYAMSWVRQAPGKGLEWVSS
IDEMGWQTYYADSVKGRFTISRDNSKNTLYLQMNSLRAEDTAVYYCAKSA
AAFDYWGQGTLVTVSS
Variable 6F8 Light Chain
(SEQ ID NO: 296)
DIQMTQSPSSLSASVGDRVTITCRASQSISSYLNWYQQKPGKAPKLLIYA
ASSLQSGVPSRFSGSGSGTDFTLTISSLQPEDFATYYCQQALVAPLTFGQ
GTKVEIKR
Variable 6F8 Heavy Chain
(SEQ ID NO: 297)
EVQLLESGGGLVQPGGSLRLSCAASGFTFSSYAMSWVRQAPGKGLEWVSS
IDEMGWQTYYADSVKGRFTISRDNSKNTLYLQMNSLRAEDTAVYYCAKSA
AAFDYWGQGTLVTVSS

By way of non-limiting example, the CM includes an amino acid sequence that is a substrate or is derived from a substrate that is cleaved by one or more of the following enzymes or proteases listed in Table 3.

TABLE 3
Exemplary Enzymes/Proteases
ADAMS, ADAMTS, e.g.
ADAM8
ADAM9
ADAM10
ADAM12
ADAM15
ADAM17/TACE
ADAMDEC1
ADAMTS1
ADAMTS4
ADAMTS5
Aspartate proteases, e.g.,
BACE
Renin
Aspartic cathepsins, e.g.,
Cathepsin D
Cathepsin E
Caspases, e.g.,
Caspase 1
Caspase 2
Caspase 3
Caspase 4
Caspase 5
Caspase 6
Caspase 7
Caspase 8
Caspase 9
Caspase 10
Caspase 14
Cysteine cathepsins, e.g.,
Cathepsin B
Cathepsin C
Cathepsin K
Cathepsin L
Cathepsin S
Cathepsin V/L2
Cathepsin X/Z/P
Cysteine proteinases, e.g.,
Cruzipain
Legumain
Otubain-2
KLKs, e.g.,
KLK4
KLK5
KLK6
KLK7
KLK8
KLK10
KLK11
KLK13
KLK14
Metallo proteinases, e.g.,
Meprin
Neprilysin
PSMA
BMP-1
MMPs, e.g.,
MMP1
MMP2
MMP3
MMP7
MMP8
MMP9
MMP10
MMP11
MMP12
MMP13
MMP14
MMP15
MMP16
MMP17
MMP19
MMP20
MMP23
MMP24
MMP26
MMP27
Serine proteases, e.g.,
activated protein C
Cathepsin A
Cathepsin G
Chymase
coagulation factor proteases
(e.g., FVIIa, FIXa, FXa, FXIa,
FXIIa)
Elastase
Granzyme B
Guanidinobenzoatase
HtrA1
Human Neutrophil Elastase
Lactoferrin
Marapsin
NS3/4A
PACE4
Plasmin
PSA
tPA
Thrombin
Tryptase
uPA
Type II Transmembrane
Serine Proteases (TTSPs), e.g.,
DESC1
DPP-4
FAP
Hepsin
Matriptase-2
MT-SP1 /Matriptase
TMPRSS2
TMPRSS3
TMPRSS4

The conjugated activatable antibodies provided herein include a masking moiety. In some embodiments, the masking moiety is an amino acid sequence that is coupled or otherwise attached to the activatable antibody and is positioned within the activatable antibody construct such that the masking moiety decreases the ability of the antibody to specifically bind the target. Suitable masking moieties are identified using any of a variety of known techniques. For example, peptide masking moieties are identified using the methods described in U.S. Pat. No. 8,293,685 by Daugherty et al., the contents of which are hereby incorporated by reference in their entirety.

In some embodiments, the masking moiety is selected for use with a specific antibody or antibody fragment. For example, suitable masking moieties for use with antibodies that bind EGFR include MMs that include the sequence CISPRG (SEQ ID NO: 29). By way of non-limiting examples, the MM can include a sequence such as CISPRGCG (SEQ ID NO: 30); CISPRGCPDGPYVMY (SEQ ID NO: 31); CISPRGCPDGPYVM (SEQ ID NO: 32), CISPRGCEPGTYVPT (SEQ ID NO: 33) and CISPRGCPGQIWHPP (SEQ ID NO: 34). Other suitable masking moieties include any of the EGFR-specific masks disclosed in PCT Publication No. WO 2010/081173, such as, by way of non-limiting example, GSHCLIPINMGAPSC (SEQ ID NO: 35); CISPRGCGGSSASQSGQGSHCLIPINMGAPSC (SEQ ID NO: 36); CNHHYFYTCGCISPRGCPG (SEQ ID NO: 37); ADHVFWGSYGCISPRGCPG (SEQ ID NO: 38); CHHVYWGHCGCISPRGCPG (SEQ ID NO: 39); CPHFTTTSCGCISPRGCPG (SEQ ID NO: 40); CNHHYHYYCGCISPRGCPG (SEQ ID NO: 41); CPHVSFGSCGCISPRGCPG (SEQ ID NO: 42); CPYYTLSYCGCISPRGCPG (SEQ ID NO: 43); CNHVYFGTCGCISPRGCPG (SEQ ID NO: 44); CNHFTLTTCGCISPRGCPG (SEQ ID NO: 45); CHHFTLTTCGCISPRGCPG (SEQ ID NO: 46); YNPCATPMCCISPRGCPG (SEQ ID NO: 47); CNHHYFYTCGCISPRGCG (SEQ ID NO: 48); CNHHYHYYCGCISPRGCG (SEQ ID NO: 49); CNHVYFGTCGCISPRGCG (SEQ ID NO: 50); CHHVYWGHCGCISPRGCG (SEQ ID NO: 51); CPHFTTTSCGCISPRGCG (SEQ ID NO: 52); CNHFTLTTCGCISPRGCG (SEQ ID NO: 53); CHHFTLTTCGCISPRGCG (SEQ ID NO: 54); CPYYTLSYCGCISPRGCG (SEQ ID NO: 55); CPHVSFGSCGCISPRGCG (SEQ ID NO: 56); ADHVFWGSYGCISPRGCG (SEQ ID NO: 57); YNPCATPMCCISPRGCG (SEQ ID NO: 58); CHHVYWGHCGCISPRGCG (SEQ ID NO: 59); C(N/P) H(H/V/F) (Y/T) (F/W/T/L) (Y/G/T/S) (T/S/Y/H) CGCISPRGCG (SEQ ID NO: 60); CISPRGCGQPIPSVK (SEQ ID NO: 61); CISPRGCTQPYHVSR (SEQ ID NO: 62); and/or CISPRGCNAVSGLGS (SEQ ID NO: 63).

Suitable masking moieties for use with antibodies that bind a Jagged target, e.g., Jagged 1 and/or Jagged 2, include, by way of non-limiting example, masking moieties that include a sequence such as QGQSGQCNIWLVGGDCRGWQG (SEQ ID NO: 232); QGQSGQGQQQWCNIWINGGDCRGWNG (SEQ ID NO: 64); PWCMQRQDFLRCPQP (SEQ ID NO: 65); QLGLPAYMCTFECLR (SEQ ID NO: 66); CNLWVSGGDCGGLQG (SEQ ID NO: 67); SCSLWTSGSCLPHSP (SEQ ID NO: 68); YCLQLPHYMQAMCGR (SEQ ID NO: 69); CFLYSCTDVSYWNNT (SEQ ID NO: 70); PWCMQRQDYLRCPQP (SEQ ID NO: 71); CNLWISGGDCRGLAG (SEQ ID NO: 72); CNLWVSGGDCRGVQG (SEQ ID NO: 73); CNLWVSGGDCRGLRG (SEQ ID NO: 74); CNLWISGGDCRGLPG (SEQ ID NO: 75); CNLWVSGGDCRDAPW (SEQ ID NO: 76); CNLWVSGGDCRDLLG (SEQ ID NO: 77); CNLWVSGGDCRGLQG (SEQ ID NO: 78); CNLWLHGGDCRGWQG (SEQ ID NO: 79); CNIWLVGGDCRGWQG (SEQ ID NO: 80); CTTWFCGGDCGVMRG (SEQ ID NO: 81); CNIWGPSVDCGALLG (SEQ ID NO: 82); CNIWVNGGDCRSFEG (SEQ ID NO: 83); YCLNLPRYMQDMCWA (SEQ ID NO: 84); YCLALPHYMQADCAR (SEQ ID NO: 85); CFLYSCGDVSYWGSA (SEQ ID NO: 86); CYLYSCTDSAFWNNR (SEQ ID NO: 87); CYLYSCNDVSYWSNT (SEQ ID NO: 88); CFLYSCTDVSYW (SEQ ID NO: 89); CFLYSCTDVAYWNSA (SEQ ID NO: 90); CFLYSCTDVSYWGDT (SEQ ID NO: 91); CFLYSCTDVSYWGNS (SEQ ID NO: 92); CFLYSCTDVAYWNNT (SEQ ID NO: 93); CFLYSCGDVSYWGNPGLS (SEQ ID NO: 94); CFLYSCTDVAYWSGL (SEQ ID NO: 95); CYLYSCTDGSYWNST (SEQ ID NO: 96); CFLYSCSDVSYWGNI (SEQ ID NO: 97); CFLYSCTDVAYW (SEQ ID NO: 98); CFLYSCTDVSYWGST (SEQ ID NO: 99); CFLYSCTDVAYWGDT (SEQ ID NO: 100); GCNIWLNGGDCRGWVDPLQG (SEQ ID NO: 101); GCNIWLVGGDCRGWIGDTNG (SEQ ID NO: 102); GCNIWLVGGDCRGWIEDSNG (SEQ ID NO: 103); GCNIWANGGDCRGWIDNIDG (SEQ ID NO: 104); GCNIWLVGGDCRGWLGEAVG (SEQ ID NO: 105); GCNIWLVGGDCRGWLEEAVG (SEQ ID NO: 106); GGPALCNIWLNGGDCRGWSG (SEQ ID NO: 107); GAPVFCNIWLNGGDCRGWMG (SEQ ID NO: 108); GQQQWCNIWINGGDCRGWNG (SEQ ID NO: 109); GKSEFCNIWLNGGDCRGWIG (SEQ ID NO: 110); GTPGGCNIWANGGDCRGWEG (SEQ ID NO: 111); GASQYCNLWINGGDCRGWRG (SEQ ID NO: 112); GCNIWLVGGDCRPWVEGG (SEQ ID NO: 113); GCNIWAVGGDCRPFVDGG (SEQ ID NO: 114); GCNIWLNGGDCRAWVDTG (SEQ ID NO: 115); GCNIWIVGGDCRPFINDG (SEQ ID NO: 116); GCNIWLNGGDCRPVVFGG (SEQ ID NO: 117); GCNIWLSGGDCRMFMNEG (SEQ ID NO: 118); GCNIWVNGGDCRSFVYSG (SEQ ID NO: 119); GCNIWLNGGDCRGWEASG (SEQ ID NO: 120); GCNIWAHGGDCRGFIEPG (SEQ ID NO: 121); GCNIWLNGGDCRTFVASG (SEQ ID NO: 122); GCNIWAHGGDCRGFIEPG (SEQ ID NO: 123); GFLENCNIWLNGGDCRTG (SEQ ID NO: 124); GIYENCNIWLNGGDCRMG (SEQ ID NO: 125); and/or GIPDNCNIWINGGDCRYG (SEQ ID NO: 126).

Suitable masking moieties for use with antibodies that bind an interleukin 6 target, e.g., interleukin 6 receptor (IL-6R), include, by way of non-limiting example, masking moieties that include a sequence such as QGQSGQYGSCSWNYVHIFMDC (SEQ ID NO: 127); QGQSGQGDFDIPFPAHWVPIT (SEQ ID NO: 128); QGQSGQMGVPAGCVWNYAHIFMDC (SEQ ID NO: 129); YRSCNWNYVSIFLDC (SEQ ID NO: 130); PGAFDIPFPAHWVPNT (SEQ ID NO: 131); ESSCVWNYVHIYMDC (SEQ ID NO: 132); YPGCKWNYDRIFLDC (SEQ ID NO: 133); YRTCSWNYVGIFLDC (SEQ ID NO: 134); YGSCSWNYVHIFMDC (SEQ ID NO: 135); YGSCSWNYVHIFLDC (SEQ ID NO: 136); YGSCNWNYVHIFLDC (SEQ ID NO: 137); YTSCNWNYVHIFMDC (SEQ ID NO: 138); YPGCKWNYDRIFLDC (SEQ ID NO: 139); WRSCNWNYAHIFLDC (SEQ ID NO: 140); WSNCHWNYVHIFLDC (SEQ ID NO: 141); DRSCTWNYVRISYDC (SEQ ID NO: 142); SGSCKWDYVHIFLDC (SEQ ID NO: 143); SRSCIWNYAHIHLDC (SEQ ID NO: 144); SMSCYWQYERIFLDC (SEQ ID NO: 145); YRSCNWNYVSIFLDC (SEQ ID NO: 146); YGSCSWNYVHIFMDC (SEQ ID NO: 147); SGSCKWDYVHIFLDC (SEQ ID NO: 148); YKSCHWDYVHIFLDC (SEQ ID NO: 149); YGSCTWNYVHIFMEC (SEQ ID NO: 150); FSSCNWNYVHIFLDC (SEQ ID NO: 151); WRSCNWNYAHIFLDC (SEQ ID NO: 152); YGSCQWNYVHIFLDC (SEQ ID NO: 153); YRSCNWNYVHIFLDC (SEQ ID NO: 154); NMSCHWDYVHIFLDC (SEQ ID NO: 155); FGPCTWNYARISWDC (SEQ ID NO: 156); XXsCXWXYvhIfXdC (SEQ ID NO: 157); MGVPAGCVWNYAHIFMDC (SEQ ID NO: 158); RDTGGQCRWDYVHIFMDC (SEQ ID NO: 159); AGVPAGCTWNYVHIFMEC (SEQ ID NO: 160); VGVPNGCVWNYAHIFMEC (SEQ ID NO: 161); DGGPAGCSWNYVHIFMEC (SEQ ID NO: 162); AVGPAGCWWNYVHIFMEC (SEQ ID NO: 163); CTWNYVHIFMDCGEGEGP (SEQ ID NO: 164); GGVPEGCTWNYAHIFMEC (SEQ ID NO: 165); AEVPAGCWWNYVHIFMEC (SEQ ID NO: 166); AGVPAGCTWNYVHIFMEC (SEQ ID NO: 167); SGASGGCKWNYVHIFMDC (SEQ ID NO: 168); MGVPAGCVWNYAHIFMDC (SEQ ID NO: 169); TPGCRWNYVHIFMECEAL (SEQ ID NO: 170); VGVPNGCVWNYAHIFMEC (SEQ ID NO: 171); PGAFDIPFPAHWVPNT (SEQ ID NO: 172); RGACDIPFPAHWIPNT (SEQ ID NO: 173); QGDFDIPFPAHWVPIT (SEQ ID NO: 174); XGafDIPFPAHWvPnT (SEQ ID NO: 175); RGDGNDSDIPFPAHWVPRT (SEQ ID NO: 176); SGVGRDRDIPFPAHWVPRT (SEQ ID NO: 177); WAGGNDCDIPFPAHWIPNT (SEQ ID NO: 178); WGDGMDVDIPFPAHWVPVT (SEQ ID NO: 179); AGSGNDSDIPFPAHWVPRT (SEQ ID NO: 180); ESRSGYADIPFPAHWVPRT (SEQ ID NO: 181); and/or RECGRCGDIPFPAHWVPRT (SEQ ID NO: 182).

In some embodiments, the masking moiety is selected for use with any antibody or antibody fragment. For example, in some embodiments, the masking moiety is a non-binding steric moiety (NB) or a binding partner (BP) for a non-binding steric moiety, where the BP recruits or otherwise attracts the NB to the activatable antibody. For example, in some embodiments, the NB is a soluble, globular protein. In some embodiments, the NB is a protein that circulates in the bloodstream. In some embodiments, the NB is selected from the group consisting of albumin, fibrinogen, fibronectin, hemoglobin, transferrin, an immunoglobulin domain, and other serum proteins. In some embodiments, the BP is selected from the group consisting of an albumin binding peptide, a fibrinogen binding peptide, a fibronectin binding peptide, a hemoglobin binding peptide, a transferrin binding peptide, an immunoglobulin domain binding peptide, and other serum protein binding peptides. In some embodiments, the activatable antibody has the structural arrangement from N-terminus to C-terminus as follows in the uncleaved state: NB-CM-AB, AB-CM-NB, BP-CM-AB or AB-CM-BP. In embodiments where the activatable antibody includes a BP and the activatable antibody is in the presence of the corresponding NB, the activatable antibody has a structural arrangement from N-terminus to C-terminus as follows in the uncleaved state: NB:BP-CM-AB or AB-CM-BP:NB, where “:” represents an interaction, e.g., binding, between the NB and BP. In some embodiments, the activatable antibody has the structural arrangement from N-terminus to C-terminus as follows in the uncleaved state: NB-LP1-CM-LP2-AB, AB-LP2-CM-LP1-NB, BP-LP1-CM-LP2-AB or AB-LP2-CM-LP1-BP. In embodiments where the activatable antibody includes a BP and the activatable antibody is in the presence of the corresponding NB, the activatable antibody has a structural arrangement from N-terminus to C-terminus as follows in the uncleaved state: NB:BP-LP1-CM-LP2-AB or AB-LP2-CM-LP1-BP:NB, where “:” represents an interaction, e.g., binding, between the NB and BP.

The conjugated activatable antibodies provided herein include a cleavable moiety. In some embodiments, the cleavable moiety includes an amino acid sequence that is a substrate for a protease, usually an extracellular protease. Suitable substrates are identified using any of a variety of known techniques. For example, peptide substrates are identified using the methods described in U.S. Pat. No. 7,666,817 by Daugherty et al., the contents of which are hereby incorporated by reference in their entirety. (See also Boulware et al. “Evolutionary optimization of peptide substrates for proteases that exhibit rapid hydrolysis kinetics.” Biotechnol Bioeng. 106.3 (2010): 339-46).

In some embodiments, the CM is selected for use with a specific protease. In some embodiments, the CM is a substrate for at least one protease selected from the group consisting of an ADAM 17, a BMP-1, a cysteine protease such as a cathepsin, a HtrAl, a legumain, a matriptase (MT-SP1), a matrix metalloprotease (MMP), a neutrophil clastase, a TMPRSS, such as TMPRSS3 or TMPRSS4, a thrombin, and a u-type plasminogen activator (uPA, also referred to as urokinase).

In some embodiments, the CM is a substrate for an ADAM17. In some embodiments, the CM is a substrate for a BMP-1. In some embodiments, the CM is a substrate for a cathepsin. In some embodiments, the CM is a substrate for a cysteine protease. In some embodiments, the CM is a substrate for a HtrAl. In some embodiments, the CM is a substrate for a legumain. In some embodiments, the CM is a substrate for a MT-SP1. In some embodiments, the CM is a substrate for a MMP. In some embodiments, the CM is a substrate for a neutrophil clastase. In some embodiments, the CM is a substrate for a thrombin. In some embodiments, the CM is a substrate for a TMPRSS. In some embodiments, the CM is a substrate for TMPRSS3. In some embodiments, the CM is a substrate for TMPRSS4. In some embodiments, the CM is a substrate for uPA.

In some embodiments, the cleavable moiety is selected for use with a specific protease, for example a protease that is known to be co-localized with the target of the activatable antibody. For example, suitable cleavable moieties for use in the activatable antibodies of the disclosure include the sequence TGRGPSWV (SEQ ID NO: 183); SARGPSRW (SEQ ID NO: 184); TARGPSFK (SEQ ID NO: 185); LSGRSDNH (SEQ ID NO: 186); GGWHTGRN (SEQ ID NO: 187); HTGRSGAL (SEQ ID NO: 188); PLTGRSGG (SEQ ID NO: 189); AARGPAIH (SEQ ID NO: 190); RGPAFNPM (SEQ ID NO: 191); SSRGPAYL (SEQ ID NO: 192); RGPATPIM (SEQ ID NO: 193); RGPA (SEQ ID NO: 194); GGQPSGMWGW (SEQ ID NO: 195); FPRPLGITGL (SEQ ID NO: 196); VHMPLGFLGP (SEQ ID NO: 197); SPLTGRSG (SEQ ID NO: 198); SAGFSLPA (SEQ ID NO: 199); LAPLGLQRR (SEQ ID NO: 200); SGGPLGVR (SEQ ID NO: 201); and/or PLGL (SEQ ID NO: 202).

In some embodiments, the CM is a substrate for at least one matrix metalloprotease (MMP). Examples of MMPs include MMP1; MMP2; MMP3; MMP7; MMP8; MMP9; MMP10; MMP11; MMP12; MMP13; MMP14; MMP15; MMP16; MMP17; MMP19; MMP20; MMP23; MMP24; MMP26; and MMP27. In some embodiments, the CM is a substrate for MMP9, MMP14, MMP1, MMP3, MMP13, MMP17, MMP11, and MMP19. In some embodiments, the CM is a substrate for MMP7. In some embodiments the CM is a substrate for MMP9. In some embodiments, the CM is a substrate for MMP14. In some embodiments, the CM is a substrate for two or more MMPs. In some embodiments, the CM is a substrate for at least MMP9 and MMP14. In some embodiments, the CM comprises two or more substrates for the same MMP. In some embodiments, the CM comprises at least two or more MMP9 substrates. In some embodiments, the CM comprises at least two or more MMP14 substrates.

In some embodiments, the CM is a substrate for an MMP and includes the sequence ISSGLLSS (SEQ ID NO: 298); QNQALRMA (SEQ ID NO: 299); AQNLLGMV (SEQ ID NO: 300); STFPFGMF (SEQ ID NO: 301); PVGYTSSL (SEQ ID NO: 302); DWLYWPGI (SEQ ID NO: 303); MIAPVAYR (SEQ ID NO: 304); RPSPMWAY (SEQ ID NO: 305); WATPRPMR (SEQ ID NO: 306); FRLLDWQW (SEQ ID NO: 307); LKAAPRWA (SEQ ID NO: 308); GPSHLVLT (SEQ ID NO: 309); LPGGLSPW (SEQ ID NO: 310); MGLFSEAG (SEQ ID NO: 311); SPLPLRVP (SEQ ID NO: 312); RMHLRSLG (SEQ ID NO: 313); LAAPLGLL (SEQ ID NO: 314); AVGLLAPP (SEQ ID NO: 315); LLAPSHRA (SEQ ID NO: 316); PAGLWLDP (SEQ ID NO: 317); and/or ISSGLSS (SEQ ID NO: 318).

In some embodiments, activatable antibodies for use in the conjugated activatable antibodies of the disclosure may be made biosynthetically using recombinant DNA technology and expression in eukaryotic or prokaryotic species. The cDNAs encoding the masking moiety, linker sequence (that may include a cleavable moiety (CM), and antibody chain (heavy or light)) can be linked in an 5′ to 3′ (N- to C-terminal in the translated product) sequence to create the nucleic acid construct, which is expressed as the activatable antibody protein following a conventional antibody expression process. In some embodiments, the activatable antibody could be semi-synthetically produced by expressing a CM-antibody and then coupling the mask chemically at or near the N-terminus of the protein. In some embodiments, the activatable antibody could be produced by expressing an antibody and then coupling the mask and the CM chemically at or near the N-terminus of the protein such that the activatable antibody in the uncleaved state has the structural arrangement from N-terminus to C-terminus as follows: MM-CM-AB or AB-CM-MM.

The conjugated activatable antibodies described herein also include an agent conjugated to the activatable antibody. In some embodiments, the conjugated agent is a therapeutic agent, such as an antineoplastic agent. In some embodiments, the agent is conjugated to a sulfhydryl group of the antibody or antigen-binding fragment in the activatable antibody. In some embodiments, the agent is a thiol-containing agent. In some embodiments, the agent is engineered to include one or more thiol groups.

In some embodiments, the agent is a cytotoxic agent such as a toxin (e.g., an enzymatically active toxin of bacterial, fungal, plant, or animal origin, or fragments thereof), or a radioactive isotope (i.e., a radioconjugate). Suitable cytotoxic agents include, for example, any of the cytotoxic agents listed in Table 4.

In some embodiments, the agent is a thiol-containing agent. In some embodiments, the agent is engineered to include one or more thiol groups. In some embodiments, the agent is a microtubule inhibitor. In some embodiments, the agent is a dolastatin or a derivative thereof (e.g. auristatin E, AFP, MMAF, MMAE, MMAD, DMAF, DMAE). In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a nucleic acid damaging agent. In some embodiments, the agent is a duocarmycin or derivative thereof. In some embodiments, the agent is a calicheamicin or derivative thereof.

In some embodiments, the agent is linked to the AB using a maleimide caproyl-valine-citrulline linker or a maleimide PEG-valine-citrulline linker. In some embodiments, the agent is linked to the AB using a maleimide caproyl-valine-citrulline linker. In some embodiments, the agent is linked to the AB using a maleimide PEG-valine-citrulline linker In some embodiments, the agent is monomethyl auristatin D (MMAD) linked to the AB using a maleimide PEG-valine-citrulline-para-aminobenzyloxycarbonyl linker, and this linker payload construct is referred to herein as “vc-MMAD.” In some embodiments, the agent is monomethyl auristatin E (MMAE) linked to the AB using a maleimide PEG-valine-citrulline-para-aminobenzyloxycarbonyl linker, and this linker payload construct is referred to herein as “vc-MMAE.” The structures of vc-MMAD and vc-MMAE are shown below:

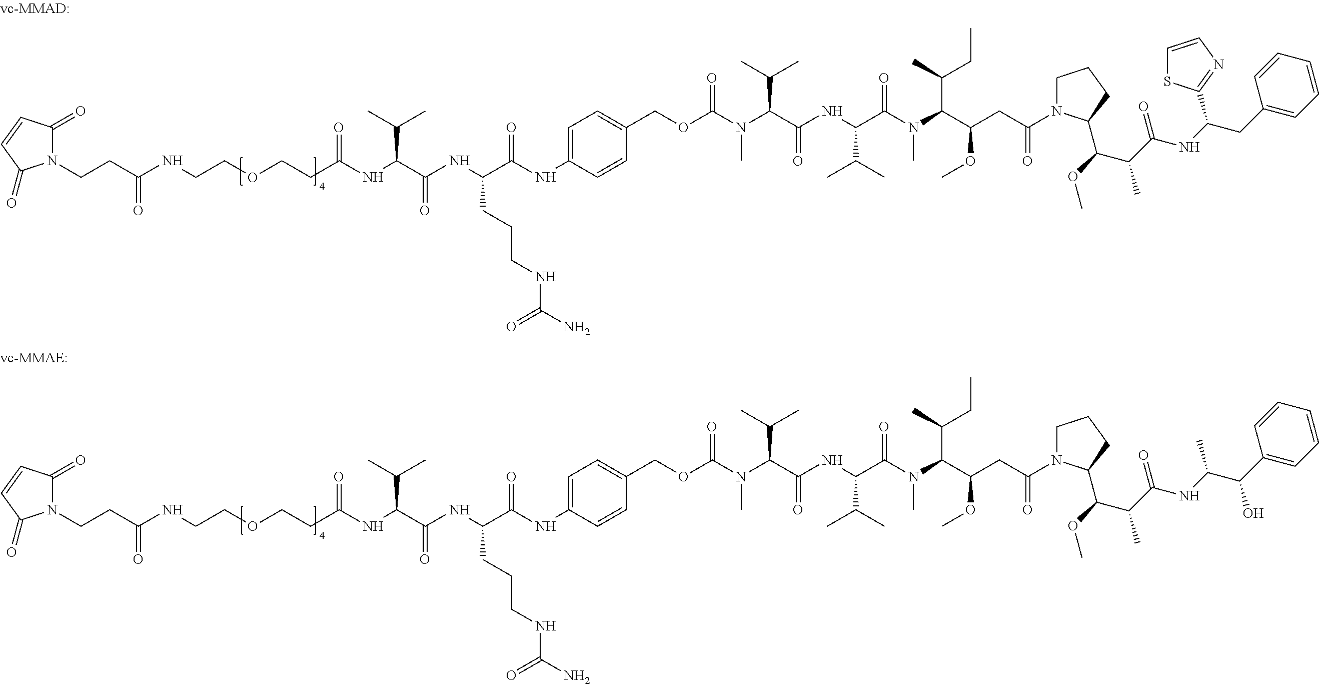

In some embodiments, in addition to the compositions and methods provided herein, the conjugated activatable antibody can also be modified for site-specific conjugation through modified amino acid sequences inserted or otherwise included in the activatable antibody sequence. These modified amino acid sequences are designed to allow for controlled placement and/or dosage of the conjugated agent within a conjugated activatable antibody. For example, the activatable antibody can be engineered to include cysteine substitutions at positions on light and heavy chains that provide reactive thiol groups and do not negatively impact protein folding and assembly, nor alter antigen binding. In some embodiments, the activatable antibody can be engineered to include or otherwise introduce one or more non-natural amino acid residues within the activatable antibody to provide suitable sites for conjugation. In some embodiments, the activatable antibody can be engineered to include or otherwise introduce enzymatically activatable peptide sequences within the activatable antibody sequence.

In some embodiments, the agent is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. (See WO94/11026).

Those of ordinary skill in the art will recognize that a large variety of possible moieties can be coupled to the resultant antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Table 4 lists some of the exemplary pharmaceutical agents that may be employed in the herein described invention but in no way is meant to be an exhaustive list.

TABLE 5
Exemplary Pharmaceutical Agents for Conjugation
CYTOTOXIC AGENTS
Auristatins
Auristatin E
Monomethyl auristatin D (MMAD)
Monomethyl auristatin E (MMAE)
Desmethyl auristatin E (DMAE)
Auristatin F
Monomethyl auristatin F (MMAF)
Desmethyl auristatin F (DMAF)
Auristatin derivatives, e.g., amides thereof
Auristatin tyramine
Auristatin quinoline
Dolastatins
Dolastatin derivatives
Dolastatin 16 DmJ
Dolastatin 16 Dpv
Maytansinoids, e.g. DM-1; DM-4
Maytansinoid derivatives
Duocarmycin
Duocarmycin derivatives
Alpha-amanitin
Anthracyclines
Doxorubicin
Daunorubicin
Bryostatins
Camptothecin
Camptothecin derivatives
7-substituted Camptothecin
10,11-
Difluoromethylenedioxycamptothecin
Combretastatins
Debromoaplysiatoxin
Kahalalide-F
Discodermolide
Ecteinascidins
ANTIVIRALS
Acyclovir
Vira A
Symmetrel
ANTIFUNGALS
Nystatin
ADDITIONAL ANTI-NEOPLASTICS
Adriamycin
Cerubidine
Bleomycin
Alkeran
Velban
Oncovin
Fluorouracil
Methotrexate
Thiotepa
Bisantrene
Novantrone
Thioguanine
Procarabizine
Cytarabine
ANTI-BACTERIALS
Aminoglycosides
Streptomycin
Neomycin
Kanamycin
Amikacin
Gentamicin
Tobramycin
Streptomycin B
Spectinomycin
Ampicillin
Sulfanilamide
Polymyxin
Chloramphenicol
Turbostatin
Phenstatins
Hydroxyphenstatin
Spongistatin 5
Spongistatin 7
Halistatin 1
Halistatin 2
Halistatin 3
Modified Bryostatins
Halocomstatins
Pyrrolobenzimidazoles (PBI)
Cibrostatin6
Doxaliform
Anthracyclins analogues
Anthracyclins analogues
Cemadotin analogue (CemCH2-SH)
Pseudomonas toxin A (PE38) variant
Pseudomonas toxin A (ZZ-PE38) variant
ZJ-101
OSW-1
4-Nitrobenzyloxycarbonyl Derivatives of
O6-Benzylguanine
Topoisomerase inhibitors
Hemiasterlin
Cephalotaxine
Homoharringtonine
Pyrrolobenzodiazepine dimers (PBDs)
Functionalized pyrrolobenzodiazepenes
Calicheamicins
Podophyllotoxins
Taxanes
Vinca alkaloids
CONJUGATABLE DETECTION
REAGENTS
Fluorescein and derivatives thereof
Fluorescein isothiocyanate (FITC)
RADIOISOTOPES
¹²⁵I
¹³¹I
⁸⁹Zr
111In
¹²³I
¹³¹I
⁹⁹mTc
²⁰¹Tl
¹³³Xe
¹¹C
⁶²Cu
¹⁸F
⁶⁸Ga
¹³N
¹⁵O
³⁸K
⁸²Rb
⁹⁹mTc (Technetium)
HEAVY METALS
Barium
Gold
Platinum
ANTI-MYCOPLASMALS
Tylosine
Spectinomycin

In some embodiments, in addition to the compositions and methods provided herein, the conjugated activatable antibody can also be coupled using any chemical reaction that will bind the two molecules so long as the antibody and the other moiety retain their respective activities. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules, such as the activatable antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Suitable linkers include: (i) SMPT (4-succinimidyloxycarbonyl-alpha-methyl-alpha-(2-pridyl-dithio)-toluene (Pierce Chem. Co., Cat. (21558G); (ii) SPDP (succinimidyl-6 [3-(2-pyridyldithio) propionamido|hexanoate (Pierce Chem. Co., Cat #21651G); and (iii) Sulfo-LC-SPDP (sulfosuccinimidyl 6 [3-(2-pyridyldithio)-propianamide]hexanoate (Pierce Chem. Co. Cat. #2165-G. Additional linkers include, but are not limited to, SMCC, sulfo-SMCC, SPDB, or sulfo-SPDB.

The linkers described above contain components that have different attributes, thus leading to conjugates with differing physio-chemical properties. For example, the linker SMPT contains a sterically hindered disulfide bond, and can form conjugates with increased stability. Disulfide linkages, are in general, less stable than other linkages because the disulfide linkage is cleaved in vitro, resulting in less conjugate available.

The reagent EDC (1-ethyl-3-(3-dimethylaminopropyl) carbodiimide hydrochloride is useful to create a carboxamide starting with a carboxylic acid and a primary or secondary amine. Thus, EDC may be used to link lysine residues in an antibody with a carboxylic acid in a linker or toxin, or to link aspartate or glutamate residues in an antibody with an amine in a linker or toxin. Such conjugation reactions utilizing EDC may be enhanced by addition of NHS (N-hydroxysuccinimide) or sulfo-NHS (N-hydroxy-3-oxysulfonylsuccinimide). Addition of NHS or sulfo-NHS to such conjugation reactions may enhance the rate, completeness, selectivity, and/or reproducibility of the conjugation reactions.

In some embodiments, the linkers are cleavable. In some embodiments, the linkers are non-cleavable. In some embodiments, two or more linkers are present. The two or more linkers are all the same, e.g., cleavable or non-cleavable, or the two or more linkers are different, e.g., at least one cleavable and at least one non-cleavable.

In some embodiments, in addition to the compositions and methods provided herein, the conjugated activatable antibody can also be further conjugated using any of several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB. According to the invention, ABs may be covalently attached to an agent through an intermediate linker having at least two reactive groups, one to react with AB and one to react with the agent. The linker, which may include any compatible organic compound, can be chosen such that the reaction with AB (or agent) does not adversely affect AB reactivity and selectivity. Furthermore, the attachment of linker to agent might not destroy the activity of the agent. Suitable linkers for reaction with oxidized antibodies or oxidized antibody fragments include those containing an amine selected from the group consisting of primary amine, secondary amine, hydrazine, hydrazide, hydroxylamine, phenylhydrazine, semicarbazide and thiosemicarbazide groups. Such reactive functional groups may exist as part of the structure of the linker, or may be introduced by suitable chemical modification of linkers not containing such groups.

According to the present invention, suitable linkers for attachment to reduced ABs include those having certain reactive groups capable of reaction with a sulfhydryl group of a reduced antibody or fragment. Such reactive groups include, but are not limited to: reactive haloalkyl groups (including, for example, haloacetyl groups), p-mercuribenzoate groups and groups capable of Michael-type addition reactions (including, for example, maleimides and groups of the type described by Mitra and Lawton, 1979, J. Amer. Chem. Soc. 101:3097-3110).

According to the present invention, suitable linkers for attachment to neither oxidized nor reduced ABs include those having certain functional groups capable of reaction with the primary amino groups present in unmodified lysine residues in the AB. Such reactive groups include, but are not limited to, NHS carboxylic or carbonic esters, sulfo-NHS carboxylic or carbonic esters, 4-nitrophenyl carboxylic or carbonic esters, pentafluorophenyl carboxylic or carbonic esters, acyl imidazoles, isocyanates, and isothiocyanates.

According to the present invention, suitable linkers for attachment to neither oxidized nor reduced ABs include those having certain functional groups capable of reaction with the carboxylic acid groups present in aspartate or glutamate residues in the AB, which have been activated with suitable reagents. Suitable activating reagents include EDC, with or without added NHS or sulfo-NHS, and other dehydrating agents utilized for carboxamide formation. In these instances, the functional groups present in the suitable linkers would include primary and secondary amines, hydrazines, hydroxylamines, and hydrazides.

The agent may be attached to the linker before or after the linker is attached to the AB. In certain applications it may be desirable to first produce an AB-linker intermediate in which the linker is free of an associated agent. Depending upon the particular application, a specific agent may then be covalently attached to the linker. In some embodiments the AB is first attached to the MM, CM and associated linkers and then attached to the linker for conjugation purposes.

Branched Linkers: In specific embodiments, branched linkers that have multiple sites for attachment of agents are utilized. For multiple site linkers, a single covalent attachment to an AB would result in an AB-linker intermediate capable of binding an agent at a number of sites. The sites may be aldehyde or sulfhydryl groups or any chemical site to which agents can be attached.

In some embodiments, higher specific activity (or higher ratio of agents to AB) can be achieved by attachment of a single site linker at a plurality of sites on the AB. This plurality of sites may be introduced into the AB by either of two methods. First, one may generate multiple aldehyde groups and/or sulfhydryl groups in the same AB. Second, one may attach to an aldehyde or sulfhydryl of the AB a “branched linker” having multiple functional sites for subsequent attachment to linkers. The functional sites of the branched linker or multiple site linker may be aldehyde or sulfhydryl groups, or may be any chemical site to which linkers may be attached. Still higher specific activities may be obtained by combining these two approaches, that is, attaching multiple site linkers at several sites on the AB.

Cleavable Linkers: Peptide linkers that are susceptible to cleavage by enzymes of the complement system, such as but not limited to urokinase, tissue plasminogen activator, trypsin, plasmin, or another enzyme having proteolytic activity may be used in one embodiment of the present invention. According to one method of the present invention, an agent is attached via a linker susceptible to cleavage by complement. The antibody is selected from a class that can activate complement. The antibody-agent conjugate, thus, activates the complement cascade and releases the agent at the target site. According to another method of the present invention, an agent is attached via a linker susceptible to cleavage by enzymes having a proteolytic activity such as a urokinase, a tissue plasminogen activator, plasmin, or trypsin. These cleavable linkers are useful in conjugated activatable antibodies that include an extracellular toxin, e.g., by way of non-limiting example, any of the extracellular toxins shown in Table 4.

Non-limiting examples of cleavable linker sequences are provided in Table 5.

TABLE 5
Exemplary Linker Sequences for Conjugation

| Types of | |
|---|---|
| Cleavable Sequences | Amino Acid Sequence |
| Plasmin | |
| cleavable sequences | |
| Pro-urokinase | PRFKIIGG (SEQ ID NO: 203) |
| | PRFRIIGG (SEQ ID NO: 204) |
| TGFβ | SSRHRRALD |
| | (SEQ ID NO: 205) |
| Plasminogen | RKSSIIIRMRDVVL |
| | (SEQ ID NO: 206) |
| Staphylokinase | SSSFDKGKYKKGDDA |
| | (SEQ ID NO: 207) |
| | SSSFDKGKYKRGDDA |
| | (SEQ ID NO: 208) |
| Factor Xa cleavable | IEGR (SEQ ID NO: 209) |
| sequences | IDGR (SEQ ID NO: 210) |
| | GGSIDGR (SEQ ID NO: 211) |
| MMP cleavable sequences | |
| Gelatinase A | PLGLWA (SEQ ID NO: 212) |
| Collagenase | |
| cleavable sequences | |
| Calf skin collagen | GPQGIAGQ (SEQ ID NO: 213) |
| (α1(I) chain) | |
| Calf skin collagen | GPQGLLGA (SEQ ID NO: 214) |
| (α2(I) chain) | |
| Bovine cartilage | GIAGQ (SEQ ID NO: 215) |
| collagen (α1(II) chain) | |
| Human liver collagen | GPLGIAGI (SEQ ID NO: 216) |
| (α1(III) chain) | |
| Human α₂M | GPEGLRVG (SEQ ID NO: 217) |
| Human PZP | YGAGLGVV (SEQ ID NO: 218) |
| | AGLGVVER (SEQ ID NO: 219) |
| | AGLGISST (SEQ ID NO: 220) |
| Rat α₁M | EPQALAMS (SEQ ID NO: 221) |
| | QALAMSAI (SEQ ID NO: 222) |
| Rat α₂M | AAYHLVSQ (SEQ ID NO: 223) |
| | MDAFLESS (SEQ ID NO: 224) |
| Rat α₁I₃(2J) | ESLPVVAV (SEQ ID NO: 225) |
| Rat α₁I₃(27J) | SAPAVESE (SEQ ID NO: 226) |
| Human fibroblast | DVAQFVLT (SEQ ID NO: 227) |
| collagenase (autolytic | VAQFVLTE (SEQ ID NO: 228) |
| cleavages) | AQFVLTEG (SEQ ID NO: 229) |
| | PVQPIGPQ (SEQ ID NO: 230) |

In addition, agents may be attached via disulfide bonds (for example, the disulfide bonds on a cysteine molecule) to the AB. Since many tumors naturally release high levels of glutathione (a reducing agent) this can reduce the disulfide bonds with subsequent release of the agent at the site of delivery. In certain specific embodiments the reducing agent that would modify a CM would also modify the linker of the conjugated activatable antibody.

Spacer Elements and Cleavable Elements: In some embodiments, it may be necessary to construct the linker in such a way as to optimize the spacing between the agent and the AB of the activatable antibody. This may be accomplished by use of a linker of the general structure:

W—(CH₂)n-Q
wherein
W is either —NH—CH₂— or —CH₂—;
Q is an amino acid, peptide; and
n is an integer from 0 to 20.

In some embodiments, the linker may comprise a spacer element and a cleavable element. The spacer element serves to position the cleavable element away from the core of the AB such that the cleavable element is more accessible to the enzyme responsible for cleavage. Certain of the branched linkers described above may serve as spacer elements.

Throughout this discussion, it should be understood that the attachment of linker to agent (or of spacer element to cleavable element, or cleavable element to agent) need not be effected by a particular mode of attachment or reaction. Any reaction providing a product of suitable stability and biological compatibility is acceptable.

Serum Complement and Selection of Linkers: According to one method of the present invention, when release of an agent is desired, an AB that is an antibody of a class that can activate complement is used. The resulting conjugate retains both the ability to bind antigen and activate the complement cascade. Thus, according to this embodiment of the present invention, an agent is joined to one end of the cleavable linker or cleavable element and the other end of the linker group is attached to a specific site on the AB. For example, if the agent has an hydroxy group or an amino group, it may be attached to the carboxy terminus of a peptide, amino acid or other suitably chosen linker via an ester or amide bond, respectively. For example, such agents may be attached to the linker peptide via a carbodimide reaction. If the agent contains functional groups that would interfere with attachment to the linker, these interfering functional groups can be blocked before attachment and deblocked once the product conjugate or intermediate is made. The opposite or amino terminus of the linker is then used either directly or after further modification for binding to an AB that is capable of activating complement.

Linkers (or spacer elements of linkers) may be of any desired length, one end of which can be covalently attached to specific sites on the AB of the activatable antibody. The other end of the linker or spacer element may be attached to an amino acid or peptide linker.

Thus when these conjugates bind to antigen in the presence of complement the amide or ester bond that attaches the agent to the linker will be cleaved, resulting in release of the agent in its active form. These conjugates, when administered to a subject, will accomplish delivery and release of the agent at the target site, and are particularly effective for the in vivo delivery of pharmaceutical agents, antibiotics, antimetabolites, antiproliferative agents and the like as presented in but not limited to those in Table 4.

Linkers for Release without Complement Activation: In yet another application of targeted delivery, release of the agent without complement activation is desired since activation of the complement cascade will ultimately lyse the target cell. Hence, this approach is useful when delivery and release of the agent should be accomplished without killing the target cell. Such is the goal when delivery of cell mediators such as hormones, enzymes, corticosteroids, neurotransmitters, genes or enzymes to target cells is desired. These conjugates may be prepared by attaching the agent to an AB that is not capable of activating complement via a linker that is mildly susceptible to cleavage by serum proteases. When this conjugate is administered to an individual, antigen-antibody complexes will form quickly whereas cleavage of the agent will occur slowly, thus resulting in release of the compound at the target site.

Biochemical Cross Linkers: In some embodiments, the activatable antibody may be conjugated to one or more therapeutic agents using certain biochemical cross-linkers. Cross-linking reagents form molecular bridges that tie together functional groups of two different molecules. To link two different proteins in a step-wise manner, hetero-bifunctional cross-linkers can be used that eliminate unwanted homopolymer formation.

Peptidyl linkers cleavable by lysosomal proteases are also useful, for example, Val-Cit, Val-Ala or other dipeptides. In addition, acid-labile linkers cleavable in the low-pH environment of the lysosome may be used, for example: bis-sialyl ether. Other suitable linkers include cathepsin-labile substrates, particularly those that show optimal function at an acidic pH. Exemplary hetero-bifunctional cross-linkers are referenced in Table 6.

TABLE 6
Exemplary Hetero-Bifunctional Cross Linkers
HETERO-BIFUNCTIONAL CROSS-LINKERS

| | | | Spacer Arm |
|---|---|---|---|
| | | | Length after |
| | | | cross-linking |
| Linker | Reactive Toward | Advantages and Applications | (Angstroms) |
| SMPT | Primary amines | Greater stability | 11.2 Å |
| | Sulfhydryls | | |
| SPDP | Primary amines | Thiolation | 6.8 Å |
| | Sulfhydryls | Cleavable cross-linking | |
| LC-SPDP | Primary amines | Extended spacer arm | 15.6 Å |
| | Sulfhydryls | | |
| Sulfo-LC-SPDP | Primary amines | Extender spacer arm | 15.6 Å |
| | Sulfhydryls | Water-soluble | |
| SMCC | Primary amines | Stable maleimide reactive | 11.6 Å |
| | | group | |
| | Sulfhydryls | Enzyme-antibody conjugation | |
| | | Hapten-carrier protein | |
| | | conjugation | |
| Sulfo-SMCC | Primary amines | Stable maleimide reactive | 11.6 Å |
| | | group | |
| | Sulfhydryls | Water-soluble | |
| | | Enzyme-antibody conjugation | |
| MBS | Primary amines | Enzyme-antibody conjugation | 9.9 Å |
| | Sulfhydryls | Hapten-carrier protein | |
| | | conjugation | |
| Sulfo-MBS | Primary amines | Water-soluble | 9.9 Å |
| | Sulfhydryls | | |
| SIAB | Primary amines | Enzyme-antibody conjugation | 10.6 Å |
| | Sulfhydryls | | |
| Sulfo-SIAB | Primary amines | Water-soluble | 10.6 Å |
| | Sulfhydryls | | |
| SMPB | Primary amines | Extended spacer arm | 14.5 Å |
| | Sulfhydryls | Enzyme-antibody conjugation | |
| Sulfo-SMPB | Primary amines | Extended spacer arm | 14.5 Å |
| | Sulfhydryls | Water-soluble | |
| EDE/Sulfo-NHS | Primary amines | Hapten-Carrier conjugation | 0 |
| | Carboxyl groups | | |
| ABH | Carbohydrates | Reacts with sugar groups | 11.9 Å |
| | Nonselective | | |

Non-Cleavable Linkers or Direct Attachment: In some embodiments of the invention, the conjugate may be designed so that the agent is delivered to the target but not released. This may be accomplished by attaching an agent to an AB either directly or via a non-cleavable linker.

These non-cleavable linkers may include amino acids, peptides, D-amino acids or other organic compounds that may be modified to include functional groups that can subsequently be utilized in attachment to ABs by the methods described herein. A-general formula for such an organic linker could be

W—(CH₂)n-Q
wherein
W is either —NH—CH₂— or —CH₂—;
Q is an amino acid, peptide; and
n is an integer from 0 to 20.

Non-Cleavable Conjugates: In some embodiments, a compound may be attached to ABs that do not activate complement. When using ABs that are incapable of complement activation, this attachment may be accomplished using linkers that are susceptible to cleavage by activated complement or using linkers that are not susceptible to cleavage by activated complement.

### Reducing Agents

Reducing agent: Examples of reducing agents suitable for use in the compositions and methods of the disclosure include, by way of non-limiting example, BMS (bis(2-mercaptoethyl) sulfone), cysteamine, cysteine, DMH (dimethyl-bis-mercaptoacetyl hydrazine), DTBA (dithiobutylamine), DTT (dithiothreitol), GILT (gamma interferon inducible lysosomal thiol reductase; for enzymatic reduction), glutathione, β-mercaptoethanol, MEA (2-mercaptoethylamine), pyridine-2-thione, sodium borohydride, sodium phosphorothioate, TCEP ((tris(2-carboxyethyl) phosphine)), and thiopropyl-agarose. In some embodiments, the reducing agent is DTT, β-mercaptoethanol or TCEP.

The studies provided herein use the reducing agent TCEP (tris(2-carboxyethyl) phosphine), which has the following structure:

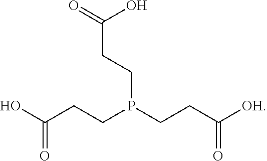 [SMILES: O=C(O)CCP(CCC(=O)O)CCC(=O)O]

TCEP is often used as a reducing agent to cleave disulfide bonds within and between proteins. TCEP is very selective and does not react toward other functional groups found within proteins. TCEP does not react with buried disulfides.

Compared to the other two most common agents used for this purpose (DTT and β-mercaptoethanol), TCEP has the advantages of being odorless, a more powerful reducing agent, an irreversible reducing agent, more hydrophilic, and more resistant to oxidation in air. Unlike DTT, TCEP is active at both alkaline and acidic conditions. TCEP is particularly useful when labeling cysteine residues with maleimides. TCEP can keep the cysteines from forming disulfide bonds, and unlike DTT and β-mercaptoethanol, it will not react as readily with the maleimide.

The ratio of reduction agent to activatable antibody will vary depending on the activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, domain antibody, single chain, Fab, and F(ab′)₂ fragments, scFvs, and an Fab expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences that are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is said to specifically bind an antigen when the dissociation constant is ≤1 μM; in some embodiments, the dissociation constant is ≤100 nM; in some embodiments, the dissociation constant is ≤10 nM.

As used herein, the terms “specific binding,” “immunological binding,” and “immunological binding properties” refer to the non-covalent interactions of the type that occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to a target, when the dissociation binding constant (K_{d}) is ≤1 μM as measured by assays such as radioligand binding assays or similar assays known to those skilled in the art. In some embodiments, the K_{d} is ≤100 nM. In some embodiments, the K_{d} is ≤ 10 nM. In some embodiments, the K_{d} is ≤1 nM. In some embodiments, the K_{d} is ≤100 pM to about 1 pM.

The compositions and methods provided herein enable the attachment of one or more agents to one or more cysteine residues in the AB without compromising the activity (e.g., the masking, activating or binding activity) of the activatable antibody.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide that it is not linked to in nature, or (3) does not occur in nature as part of a larger sequence. Polynucleotides in accordance with the invention include the nucleic acid molecules encoding the heavy chain immunoglobulin molecules shown herein, and nucleic acid molecules encoding the light chain immunoglobulin molecules shown herein.

The term “isolated protein” referred to herein means a protein expressed from cDNA or recombinant RNA, or a protein of synthetic origin or some combination thereof, which by virtue of its origin, or source of derivation, the “isolated protein” (1) is not associated with proteins found in nature, (2) is free of other proteins from the same source, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules shown herein, and the light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences that are necessary to effect the expression and processing of coding sequences to which they are ligated. The nature of such control sequences differs depending upon the host organism: in prokaryotes and eukaryotes, such control sequences generally include promoter, ribosomal binding site, and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In some embodiments, oligonucleotides are 10 to 60 bases in length. In some embodiments, the oligonucleotides are 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the invention are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

As used herein, the twenty conventional amino acids and their abbreviations follow conventional usage. See Immunology—A Synthesis (2nd Edition, E. S. Golub and D. R. Gren, Eds., Sinauer Associates, Sunderland 7 Mass. (1991)). Stercoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction. Sequence regions on the DNA strand having the same sequence as the RNA and that are 5′ to the 5′ end of the RNA transcript are referred to as “upstream sequences”. Sequence regions on the DNA strand having the same sequence as the RNA and that are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity. In some embodiments, the two peptide sequences share at least 90 percent sequence identity. In some embodiments, the two peptide sequences share at least 95 percent sequence identity. In some embodiments, the two peptide sequences share at least 99 percent sequence identity.

In some embodiments, residue positions that are not identical differ by conservative amino acid substitutions.

As discussed herein, minor variations in the amino acid sequences of antibodies or immunoglobulin molecules are contemplated as being encompassed by the present invention, providing that the variations in the amino acid sequence maintain at least 75% amino acid sequence identity to a reference sequence (e.g., the wild-type sequence). In some embodiments, the variations in the amino acid sequence maintain at least 80%, 90%, 95%, or 99% amino acid identity to the reference sequence. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or other properties of the resulting molecule, for example, in situations where the replacement does not involve an amino acid within a complementarity determining region (CDR) or other variable region. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. In some embodiments, amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

In some embodiments, amino acid substitutions are those that: (1) decrease susceptibility to proteolysis, (2) decrease susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (in some embodiments, conservative amino acid substitutions) may be made in the naturally-occurring sequence (in some embodiments, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion and/or one or more internal deletion(s), but where the remaining amino acid sequence is identical to the corresponding positions in the naturally-occurring sequence deduced, for example, from a full length cDNA sequence. Fragments typically are at least 2, 3, 4, 5, 6, 7, 8, 9 or 10 amino acids long. In some embodiments, the fragment is an antibody fragment that is at least 14 amino acids long. In some embodiments, the fragment is a fragment of the AB that is least 20 amino acids long. In some embodiments, the fragment is a fragment of the AB that is at least 50 amino acids long. In some embodiments, the fragment is a fragment of the AB that is at least 70 amino acids long. The term “analog” as used herein refers to polypeptides that are comprised of a segment of at least 25 amino acids that has substantial identity to a portion of a deduced amino acid sequence and that has specific binding to a target, under suitable binding conditions. Typically, polypeptide analogs comprise a conservative amino acid substitution (or addition or deletion) with respect to the naturally-occurring sequence. Analogs typically are at least 20 amino acids long, in some embodiments, at least 50 amino acids long or longer, and can often be as long as a full-length naturally-occurring polypeptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to decrease potential steric hindrance. The term “pharmaceutical agent or drug” as used herein refers to a chemical compound or composition capable of inducing a desired therapeutic effect when properly administered to a patient.

The term “drug” as used herein means an element, compound, agent, or molecular entity, including, e.g., a pharmaceutical, therapeutic, or pharmacologic compound. Drugs can be natural or synthetic or a combination thereof. A “therapeutic drug” is an agent that exerts a therapeutic (e.g., beneficial) effect on cancer cells or immune cells (e.g., activated immune cells), cither alone or in combination with another agent (e.g., a prodrug converting enzyme in combination with a prodrug). Typically, therapeutic drugs useful in accordance with the methods and compositions described herein are those that exert a cytotoxic, cytostatic, or immunosuppressive effect. In certain embodiments, a drug is not a radioactive element. The drug can be a thiol-containing agent and/or the drug can be engineered to include one or more thiol groups.

“Cytotoxic agent,” in reference to the effect of an agent on a cell, means killing of the cell. “Cytostatic agent” means an inhibition of cell proliferation.

The term “interchain disulfide bond,” in the context of an antibody, refers to a disulfide bond between two heavy chains, or a heavy and a light chain.

The term “interchain thiol” refers to a thiol group of an antibody heavy or light chain that can participate in the formation of an interchain disulfide bond.

A protein is referred to as “fully-loaded” when all points of conjugation of a particular type and/or of similar reactivity are conjugated to drugs, resulting in a homogeneous population of protein-drug conjugate. A protein is referred to as “partially-loaded” when only some of the possible points of conjugation of a particular type and/or of a similar reactivity are conjugated to drugs, resulting in formation of a certain isomer or isomers of the protein-drug conjugate.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term patient includes human and veterinary subjects.

### Use of Conjugated Activatable Antibodies

It will be appreciated that administration of conjugated activatable antibodies in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include a conjugated activatable antibody, are used to prevent, treat or otherwise ameliorate a disease or disorder associated with expression and/or activity of a target. For example, therapeutic formulations of the invention are used to treat or otherwise ameliorate a cancer or other neoplastic condition. In some embodiments the cancer is a solid tumor or a hematologic malignancy where the target is expressed. In some embodiments the cancer is a solid tumor where the target is expressed. In some embodiments the cancer is a hematologic malignancy where the target is expressed. In some embodiments, the target is expressed on parenchyma (e.g., in cancer, the portion of an organ or tissue that often carries out function(s) of the organ or tissue). In some embodiments, the target is expressed on a cell, tissue, or organ. In some embodiments, the target is expressed on stroma (i.e., the connective supportive framework of a cell, tissue, or organ). In some embodiments, the target is expressed on an osteoblast. In some embodiments, the target is expressed on the endothelium (vasculature). In some embodiments, the target is expressed on a cancer stem cell. In some embodiments, the agent to which the activatable antibody is conjugated is a microtubule inhibitor. In some embodiments, the agent to which the activatable antibody is conjugated is a nucleic acid damaging agent.

Pathologies treated and/or prevented and/or for which the progression is delayed and/or for which a symptom is ameliorated using the conjugated activatable anti-EGFR antibodies of the invention include, for example, diseases or disorders associated with expression and/or activity of EGFR. In some embodiments, the disease or disorder associated with expression and/or activity of EGFR is a cancer. In some embodiments, the cancer is a breast cancer, e.g., by way of non-limiting example, the breast cancer is a triple-negative breast cancer. In some embodiments, the cancer is a triple-negative breast cancer. In some embodiments, the cancer is colorectal cancer. In some embodiments, the cancer is gastric cancer. In some embodiments, the cancer is glioblastoma. In some embodiments, the cancer is a head and neck cancer, e.g., by way of non-limiting example, esophageal cancer. In some embodiments, the cancer is an esophageal cancer. In some embodiments, the cancer is a lung cancer, e.g., by way of non-limiting example, non-small cell lung cancer. In some embodiments, the cancer is a non-small cell lung cancer. In some embodiments, the cancer is ovarian/endometrial cancer. In some embodiments, the cancer is ovarian cancer. In some embodiments, the cancer is endometrial cancer. In some embodiments, the cancer is pancreatic cancer. In some embodiments, the cancer is prostate cancer. In some embodiments, the cancer is a renal cancer. In some embodiments, the cancer is a sarcoma, e.g., by way of non-limiting example, osteosarcoma. In some embodiments, the cancer is an osteosarcoma. In some embodiments, the cancer is a skin cancer, e.g., by way of non-limiting example, squamous cell cancer, basal cell carcinoma, and/or melanoma. In some embodiments, the cancer is a squamous cell cancer. In some embodiments, the cancer is a skin squamous cell carcinoma. In some embodiments, the cancer is an esophageal squamous cell carcinoma. In some embodiments, the cancer is a head and neck squamous cell carcinoma. In some embodiments, the cancer is a lung squamous cell carcinoma. In some embodiments, the cancer is a basal cell carcinoma. In some embodiments, the cancer is a melanoma. In some embodiments, the agent to which the activatable antibody is conjugated is a microtubule inhibitor. In some embodiments, the agent to which the activatable antibody is conjugated is a nucleic acid damaging agent.

In some embodiments, the indication, e.g., disease or disorder associated with expression and/or activity of EGFR is an inflammatory disorder and/or an autoimmune disease. In some embodiments, the inflammatory and/or autoimmune disease is psoriasis. In some embodiments, the agent to which the activatable antibody is conjugated is a microtubule inhibitor. In some embodiments, the agent to which the activatable antibody is conjugated is a nucleic acid damaging agent.

Pathologies treated and/or prevented and/or for which the progression is delayed and/or for which a symptom is ameliorated using the conjugated activatable anti-Jagged antibodies of the invention include, for example, cancer. In some embodiments, the conjugated activatable anti-Jagged antibodies of the invention are used to treat, prevent, delay the progression of, and/or ameliorate a symptom of a pathology such as, for example, leukemias, including T-cell acute lymphoblastic leukemia (T-ALL) and chronic lymphocytic leukemia (CLL), lymphoblastic diseases including multiple myeloma, and solid tumors, including lung, colorectal, prostate, pancreatic and breast, including triple negative breast cancer. In addition, since notch signaling is important for the survival and growth of cancer stem cells, inhibition of Jagged dependent notch signaling would impact stem cell growth and survival.

In some embodiments, the conjugated activatable anti-Jagged antibodies of the invention are used to treat, prevent, delay the progression of, and/or ameliorate a symptom of a pathology such as, for example, bone disease or metastasis in cancer, regardless of primary tumor origin.

In some embodiments, the conjugated activatable anti-Jagged antibodies of the invention are used to treat, prevent, delay the progression of, and/or ameliorate a symptom of a pathology such as, for example, breast cancer, including by way of non-limiting example, ER/PR+ breast cancer, Her2+ breast cancer, triple-negative breast cancer.

In some embodiments, the conjugated activatable anti-Jagged antibodies of the invention are used to treat, prevent, delay the progression of, and/or ameliorate a symptom of a pathology such as, for example, colorectal cancer.

In some embodiments, the conjugated activatable anti-Jagged antibodies of the invention are used to treat, prevent, delay the progression of, and/or ameliorate a symptom of a pathology such as, for example, gastric cancer.

In some embodiments, the conjugated activatable anti-Jagged antibodies of the invention are used to treat, prevent, delay the progression of, and/or ameliorate a symptom of a pathology such as, for example, glioblastoma.

In some embodiments, the conjugated activatable anti-Jagged antibodies of the invention are used to treat, prevent, delay the progression of, and/or ameliorate a symptom of a pathology such as, for example, head and neck cancer.

In some embodiments, the conjugated activatable anti-Jagged antibodies of the invention are used to treat, prevent, delay the progression of, and/or ameliorate a symptom of a pathology such as, for example, lung cancer, such as by way of non-limiting example, non-small cell lung cancer.

In some embodiments, the conjugated activatable anti-Jagged antibodies of the invention are used to treat, prevent, delay the progression of, and/or ameliorate a symptom of a pathology such as, for example, multiple myeloma.

In some embodiments, the conjugated activatable anti-Jagged antibodies of the invention are used to treat, prevent, delay the progression of, and/or ameliorate a symptom of a pathology such as, for example, ovarian cancer.

In some embodiments, the conjugated activatable anti-Jagged antibodies of the invention are used to treat, prevent, delay the progression of, and/or ameliorate a symptom of a pathology such as, for example, pancreatic cancer.

In some embodiments, the conjugated activatable anti-Jagged antibodies of the invention are used to treat, prevent, delay the progression of, and/or ameliorate a symptom of a pathology such as, for example, prostate cancer.

In some embodiments, the conjugated activatable anti-Jagged antibodies of the invention are used to treat, prevent, delay the progression of, and/or ameliorate a symptom of a pathology such as, for example, sarcoma.

In some embodiments, the conjugated activatable anti-Jagged antibodies of the invention are used to treat, prevent, delay the progression of, and/or ameliorate a symptom of a pathology such as, for example, renal cancer, such as by way of nonlimiting example, renal cell carcinoma.

In some embodiments, the conjugated activatable anti-Jagged antibodies of the invention are used to treat, prevent, delay the progression of, and/or ameliorate a symptom of a pathology such as, for example, thyroid cancer.

In some embodiments, the conjugated activatable anti-Jagged antibodies of the invention are used to treat, prevent, delay the progression of, and/or ameliorate a symptom of a pathology such as, for example, a urogenital cancer, such as bladder cancer, kidney cancer, or uterine cancer. In some embodiments, the pathology is bladder cancer. In some embodiments, the pathology is kidney cancer. In some embodiments, the pathology is uterine cancer.

In some embodiments, the conjugated activatable anti-Jagged antibodies of the invention are used to treat, prevent, delay the progression of, and/or ameliorate a symptom of a pathology such as, for example, skin cancer, such as by way of nonlimiting example, skin squamous cell cancer, such as esophageal squamous cell carcinoma (also known as squamous cell cancer of the esophagus), head and neck squamous cell carcinoma (also known as squamous cell cancer of the head and neck) or lung squamous cell carcinoma (also known as squamous cell cancer of the lung), basal cell carcinoma, or melanoma. In some embodiments, the cancer is a squamous cell cancer. In some embodiments, the cancer is a skin squamous cell carcinoma. In some embodiments, the cancer is an esophageal squamous cell carcinoma. In some embodiments, the cancer is a head and neck squamous cell carcinoma. In some embodiments, the cancer is a lung squamous cell carcinoma. In some embodiments, the cancer is a basal cell carcinoma. In some embodiments, the cancer is a melanoma.

In some embodiments, the conjugated activatable anti-Jagged antibodies of the invention are used to treat, prevent, delay the progression of, and/or ameliorate a symptom of a pathology such as, for example, acute lymphoblastic leukemia (ALL), acute myelogenous leukemia (AML), chronic lymphoblastic leukemia (CLL) or myelodysplastic syndrome (MDS). In some embodiments, the pathology is ALL. In some embodiments, the pathology is AML. In some embodiments, the pathology is CLL. In some embodiments, the pathology is MDS.

In addition to cancer, Jagged-dependent notch signaling is critical to epithelial and fibroblast differentiation to myofibroblasts, cells with a central role in the development of fibrotic disease. Inhibition of Jagged dependent notch signaling, and therefore inhibition of the emergence of myofibroblasts, would be an effective treatment for fibrotic diseases of the kidney, liver, lung, and skin. In some embodiments, the conjugated activatable anti-Jagged antibodies are used to treat a fibrotic disorder, such as idiopathic pulmonary fibrosis (IPF).

In some embodiments, the conjugated activatable anti-Jagged antibodies of the invention are used to treat, prevent, delay the progression of, and/or ameliorate a symptom of a pathology such as, for example, fibrotic disease.

In some embodiments, the conjugated activatable anti-Jagged antibodies of the invention are used to treat, prevent, delay the progression of, and/or ameliorate a symptom of a pathology such as, for example, idiopathic pulmonary fibrosis, kidney fibrotic disease, liver fibrotic disease, peritoneal dialysis-induced fibrosis, scleroderma.

In some embodiments, the conjugated activatable anti-Jagged antibodies of the invention are used to treat, prevent, delay the progression of, and/or ameliorate a symptom of a pathology such as, for example, hearing loss.

In some embodiments, the conjugated activatable anti-Jagged antibodies of the invention that are used to treat, prevent, delay the progression of, and/or ameliorate a symptom of such pathologies are conjugated to a microtubule inhibitor agent. In some embodiments, the conjugated activatable anti-Jagged antibodies of the invention that are used to treat, prevent, delay the progression of, and/or ameliorate a symptom of such pathologies are conjugated to a nucleic acid damaging agent.

Pathologies treated and/or prevented and/or for which the progression is delayed and/or for which a symptom is ameliorated using the conjugated activatable anti-interleukin 6 receptor (IL-6) antibodies of the invention include, for example, diseases or disorders associated with expression and/or activity of IL-6R. In some embodiments, the disease or disorder associated with expression and/or activity of IL-6R is cancer. In some embodiments, the cancer is breast cancer, including but not limited to, triple negative breast cancer (TNBC). In some embodiments, the cancer is Castleman's disease. In some embodiments, the cancer is hepatocellular carcinoma. In some embodiments, the cancer is lung cancer. In some embodiments, the cancer is multiple myeloma. In some embodiments, the cancer is ovarian cancer. In some embodiments, the cancer is prostate cancer. In some embodiments, the agent to which the activatable antibody is conjugated is a microtubule inhibitor. In some embodiments, the agent to which the activatable antibody is conjugated is a nucleic acid damaging agent.

In some embodiments, the disease or disorder is inflammation and/or an inflammatory disorder. In some embodiments, the disease or disorder is an autoimmune disease. In some embodiments, the agent to which the activatable antibody is conjugated is a microtubule inhibitor. In some embodiments, the agent to which the activatable antibody is conjugated is a nucleic acid damaging agent.

Increased proteolysis is known to be a hallmark of cancer. (See e.g., Affara N I, et al. “Delineating protease functions during cancer development.” Methods Mol Biol. 539 (2009): 1-32). Progression, invasion and metastasis of tumors result from several interdependent processes in which proteases are implicated.

Efficaciousness of prevention, amelioration or treatment is determined in association with any known method for diagnosing or treating the disease or disorder associated with target expression and/or activity. Prolonging the survival of a subject or otherwise delaying the progression of the disease or disorder associated with target expression and/or activity in a subject indicates that the activatable antibody confers a clinical benefit.

Conjugated activatable antibodies can be administered in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

One embodiment of an activatable antibody fragment is the smallest fragment that specifically binds to the binding domain of the target protein. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90:7889-7893 (1993)). The formulation can also contain more than one active compound as necessary for the particular indication being treated, and in some embodiments, those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, where matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, the activatable antibody contains a detectable label. An intact antibody, or a fragment thereof (e.g., Fab, scFv, or F (ab) 2) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect a protein, polypeptide or peptide in a biological sample in vitro as well as in vivo. For example, in vitro techniques for detection of an analyte protein include enzyme linked immunosorbent assays (ELISAs), Western blots, immunoprecipitations, immunochemical staining, and immunofluorescence. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Diagnostic and Prophylactic Formulations

The conjugated activatable antibodies of the invention are used in diagnostic and prophylactic formulations. In one embodiment, a conjugated activatable antibody is administered to patients that are at risk of developing one or more of the aforementioned cancer or other disorders. A patient's or organ's predisposition to one or more of the aforementioned disorders can be determined using genotypic, serological or biochemical markers.

In some embodiments of the invention, a conjugated activatable antibody is administered to human individuals diagnosed with a clinical indication associated with one or more of the aforementioned disorders. Upon diagnosis, a conjugated activatable antibody is administered to mitigate or reverse the effects of the clinical indication.

Conjugated activatable antibodies of the invention are also useful in the detection of a target in patient samples and accordingly are useful as diagnostics. For example, the conjugated activatable antibodies of the invention are used in in vitro assays, e.g., ELISA, to detect target levels in a patient sample.

Conjugated activatable antibodies can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, a conjugated activatable antibody having an enzymatically cleavable CM can be used to detect the presence or absence of an enzyme that is capable of cleaving the CM. Such conjugated activatable antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated conjugated activatable antibodies (i.e., antibodies resulting from cleavage of a conjugated activatable antibody) in a given cell or tissue of a given host organism. Such accumulation of activated conjugated antibodies indicates not only that the tissue expresses enzymatic activity (or an increased reduction potential depending on the nature of the CM) but also that the tissue expresses target to which the activated antibody binds.

For example, the CM can be selected to be a protease substrate for a protease found at the site of a tumor, at the site of a viral or bacterial infection at a biologically confined site (e.g., such as in an abscess, in an organ, and the like), and the like. The AB can be one that binds a target antigen. Using methods as disclosed herein or, when appropriate, methods familiar to one skilled in the art, a detectable label (e.g., a fluorescent label or radioactive label or radiotracer) can be conjugated to an AB or other region of an activatable antibody. Suitable detectable labels are discussed in the context of the above screening methods and additional specific examples are provided below. Using an AB specific to a protein or peptide of the disease state, along with a protease whose activity is elevated in the disease tissue of interest, protease-activated activatable antibodies will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, conjugated activatable antibodies can be used to detect the presence or absence of a cleaving agent in a sample. For example, where the conjugated activatable antibodies contain a CM susceptible to cleavage by an enzyme, the conjugated activatable antibodies can be used to detect (either qualitatively or quantitatively) the presence of an enzyme in the sample. In another example, where the conjugated activatable antibodies contain a CM susceptible to cleavage by reducing agent, the conjugated activatable antibodies can be used to detect (either qualitatively or quantitatively) the presence of reducing conditions in a sample. To facilitate analysis in these methods, the conjugated activatable antibodies can be detectably labeled and can be bound to a support (e.g., a solid support, such as a slide or bead). The detectable label can be positioned on a portion of the activatable antibody that is not released following cleavage, for example, the detectable label can be a quenched fluorescent label or other label that is not detectable until cleavage has occurred. The assay can be conducted by, for example, contacting the immobilized, detectably labeled activatable antibodies with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the activatable antibodies prior to contacting with the sample e.g., the presence of and/or an increase in detectable signal due to cleavage of the activatable antibody by the cleaving agent in the sample.

Such detection methods can be adapted to also provide for detection of the presence or absence of a target that is capable of binding the AB of the conjugated activatable antibodies when cleaved. Thus, the assays can be adapted to assess the presence or absence of a cleaving agent and the presence or absence of a target of interest. The presence or absence of the cleaving agent can be detected by the presence of and/or an increase in detectable label of the activatable antibodies as described above, and the presence or absence of the target can be detected by detection of a target-AB complex e.g., by use of a detectably labeled anti-target antibody.

Conjugated activatable antibodies are also useful in in situ imaging for the validation of activatable antibody activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an activatable antibody is labeled with a detectable label. The detectable label may be a fluorescent dye, (e.g. Fluorescein Isothiocyanate (FITC), Rhodamine Isothiocyanate (TRITC), an Alexa Fluor® label, such as Alexa Fluor® 680 or Alexa Fluor® 750), a near infrared (NIR) dye (e.g., Qdot® nanocrystals), a colloidal metal, a hapten, a radioactive marker, biotin and an amplification reagent such as streptavidin, or an enzyme (e.g., horseradish peroxidase or alkaline phosphatase).

Detection of the label in a sample that has been incubated with the labeled, activatable antibody indicates that the sample contains the target and contains a protease that is specific for the CM of the activatable antibody. In some embodiments, the presence of the protease can be confirmed using broad spectrum protease inhibitors and/or using an agent that is specific for the protease, for example, an antibody such as A11, which is specific for the protease matriptase (MT-SP1) and inhibits the proteolytic activity of MT-SP1; see e.g., International Publication Number WO 2010/129609, published 11 Nov. 2010. The same approach of using broad spectrum protease inhibitors and/or by using a more selective inhibitory agent can be used to identify a protease or class of proteases specific for the CM of the activatable antibody. In some embodiments, the presence of the target can be confirmed using an agent that is specific for the target, e.g., another antibody, or the detectable label can be competed with unlabeled target. In some embodiments, unlabeled activatable antibody could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

In some embodiments, in situ imaging and/or in vivo imaging are useful in methods to identify which patients to treat. For example, in in situ imaging, the activatable antibodies are used to screen patient samples to identify those patients having the appropriate protease(s) and target(s) at the appropriate location, e.g., at a tumor site.

In some embodiments in situ imaging is used to identify or otherwise refine a patient population suitable for treatment with a conjugated activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative for either or both of the target and the protease that cleaves the substrate in the CM in the activatable antibody being tested using these methods might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the conjugated activatable antibody for which the patient tested positive.

In some embodiments in vivo imaging is used to identify or otherwise refine a patient population suitable for treatment with an activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the conjugated activatable antibody for which the patient tested positive.

### Pharmaceutical Compositions

The conjugated activatable antibodies of the invention (also referred to herein as “active compounds”), and derivatives, fragments, analogs and homologs thereof, can be incorporated into pharmaceutical compositions suitable for administration. Such compositions typically comprise the conjugated activatable antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be suitable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXAMPLES

### Example 1. Materials and Methods

The examples provided herein use an anti-EGFR activatable antibody referred to herein as activatable antibody 3954-1204-C225v5 (also referred to herein as 3954-1204-C225v5 activatable antibody or 3954-1204-C225v5) that includes an EGFR-binding sequence, a masking moiety (MM), and a cleavable moiety (CM) that is a substrate for a protease. These examples also use an activatable anti-EGFR antibody construct referred to herein as masked antibody 3954-NSUB-C225v5 (also referred to herein as 3954-NSUB-C225v5 masked antibody or 3954-NSUB-C225v5) that includes a non-cleavable moiety located between the MM and the EGFR-binding sequence. It is to be understood that while the examples provided herein use these anti-EGFR activatable antibody constructs, these methods are applicable to any activatable antibody having two or more cysteine residues, where it is desired that only a portion of the total number of cysteine residues in the activatable antibody be reduced prior to conjugation. This is referred to herein as “partial reduction.”

It should be further understood that the examples provided herein use a fluorescent agent, Alexa-680 Fluor® (also referred to herein as Alexa 680®), as the agent that is to be conjugated to an activatable antibody. This particular dye was chosen because it has a molecular weight that is similar to a known cytotoxic agent, MMAE. However, this fluorescent agent is merely used as an example, and the compositions and methods used herein are useful with any number of conjugated agents, including by way of non-limiting example, toxins and other payload agents. The compositions and methods are not limited to agents of any particular molecular weight, size or other such characteristic.

Anti-EGFR activatable antibody constructs: The 3954-1204-C225v5 activatable anti-EGFR antibody construct includes the following heavy and light chain sequences:

3954-1204-C225v5 Activatable Antibody Heavy
Chain Nucleotide Sequence:
[C225v5 (SEQ ID NO: 1)]
(SEQ ID NO: 1)
[caggtgcagctgaaacagagcggcccgggcctggtgcagccgagccaga
gcctgagcattacctgcaccgtgagcggctttagcctgaccaactatggc
gtgcattgggtgcgccagagcccgggcaaaggcctggaatggctgggcgt
gatttggagcggcggcaacaccgattataacaccccgtttaccagccgcc
tgagcattaacaaagataacagcaaaagccaggtgttttttaaaatgaac
agcctgcaaagccaggataccgcgatttattattgcgcgcgcgcgctgac
ctattatgattatgaatttgcgtattggggccagggcaccctggtgaccg
tgagcgcggctagcaccaagggcccatcggtcttccccctggcaccctcc
tccaagagcacctctgggggcacagcggccctgggctgcctggtcaagga
ctacttccccgaaccggtgacggtgtcgtggaactcaggcgccctgacca
gcggcgtgcacaccttcccggctgtcctacagtcctcaggactctactcc
ctcagcagcgtggtgaccgtgccctccagcagcttgggcacccagaccta
catctgcaacgtgaatcacaagcccagcaacaccaaggtggacaagaaag
ttgagcccaaatcttgtgacaaaactcacacatgcccaccgtgcccagca
cctgaactcctggggggaccgtcagtcttcctcttccccccaaaacccaa
ggacaccctcatgatctcccggacccctgaggtcacatgcgtggtggtgg
acgtgagccacgaagaccctgaggtcaagttcaactggtacgtggacggc
gtggaggtgcataatgccaagacaaagccgcgggaggagcagtacaacag
cacgtaccgtgtggtcagcgtcctcaccgtcctgcaccaggactggctga
atggcaaggagtacaagtgcaaggtctccaacaaagccctcccagccccc
atcgagaaaaccatctccaaagccaaagggcagccccgagaaccacaggt
gtacaccctgcccccatcccgggatgaactgaccaagaaccaggtcagcc
tgacctgcctggtcaaaggcttctatcccagcgacatcgccgtggagtgg
gagagcaatgggcagccggagaacaactacaagaccacgcctcccgtgct
ggactccgacggctccttcttcctctacagcaagctcaccgtggacaaga
gcaggtggcagcaggggaacgtcttctcatgctccgtgatgcatgaggct
ctgcacaaccactacacgcagaagagcctctccctgtctccgggtaaatg
a]
3954-1204-C225v5 Activatable Antibody Heavy
Chain Amino Acid Sequence:
[C225v5 (SEQ ID NO: 2)]
(SEQ ID NO: 2)
[QVQLKQSGPGLVQPSQSLSITCTVSGFSLTNYGVHWVRQSPGKGLEWLG
VIWSGGNTDYNTPFTSRLSINKDNSKSQVFFKMNSLQSQDTAIYYCARAL
TYYDYEFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K*]
3954-1204-C225v5 Activatable Antibody Light
Chain Nucleotide Sequence:
[Spacer (SEQ ID NO: 5)] [Mask (SEQ ID NO: 6)]
[Linker 1 (SEQ ID NO: 7)] [1204 Substrate (SEQ ID
NO: 8)] [Linker 2 (SEQ ID NO: 9)] [C225
(SEQ ID NO: 10)]
(SEQ ID NO: 3)
[caaggccagtctggccag][tgcatctcacctcgtggttgtccggacgg
cccatacgtcatgtac][ggctcgagcggtggcagcggtggctctggtgg
atccggt][ctgagcggccgttccgataatcat]
[ggcagtagcggtacc][cagatcttgctgacccagagcccggtgatt
ctgagcgtgagcccgggcgaacgtgtgagctttagctgccgcgcgagcca
gagcattggcaccaacattcattggtatcagcagcgcaccaacggcagcc
cgcgcctgctgattaaatatgcgagcgaaagcattagcggcattccgagc
cgctttagcggcagcggcagcggcaccgattttaccctgagcattaacag
cgtggaaagcgaagatattgcggattattattgccagcagaacaacaact
ggccgaccacctttggcgcgggcaccaaactggaactgaaacgtacggtg
gctgcaccatctgtcttcatcttcccgccatctgatgagcagttgaaatc
tggaactgcctctgttgtgtgcctgctgaataacttctatcccagagagg
ccaaagtacagtggaaggtggataacgccctccaatcgggtaactcccag
gagagtgtcacagagcaggacagcaaggacagcacctacagcctcagcag
caccctgacgctgagcaaagcagactacgagaaacacaaagtctacgcct
gcgaagtcacccatcagggcctgagctcgcccgtcacaaagagcttcaac
aggggagagtgttag]

| | |
|---|---|
| Bold: | Spacer |
| Underline: | Mask |
| Italics and Underline: | Linker 1 |
| Bold and Underline: | 1204 Substrate |
| Italics and Underline: | Linker 2 |
| Normal text: | anti-EGFR antibody |
| | derived sequence |

3954-1204-C225v5 Activatable Antibody Light
Chain Amino Acid Sequence:
[Spacer (SEQ ID NO: 11)] [Mask (SEQ ID NO: 12)]
[Linker 1 (SEQ ID NO: 13)] [1204 Substrate (SEQ ID
NO: 14)] [Linker 2 (SEQ ID NO: 15)] [C225
(SEQ ID NO: 16)]
(SEQ ID NO: 4)
[QGQSGQ][CISPRGCPDGPYVMY][GSSGGSGGSGGSG][LSGRSDNH]
[GSSGT][QILLTQSPVILSVSPGERVSFSCRASQSIGTNIHWYQQRTNG
SPRLLIKYASESISGIPSRFSGSGSGTDFTLSINSVESEDIADYYCQQNN
NWPTTFGAGTKLELKRTVAAPSVFIFPPSDEQLKSGTASVVCLLNNFYPR
EAKVQWKVDNALQSGNSQESVTEQDSKDSTYSLSSTLTLSKADYEKHKVY
ACEVTHQGLSSPVIKSFNRGEC*]

| | |
|---|---|
| Bold: | Spacer |
| Underline: | Mask |
| Italics and Underline: | Linker 1 |
| Bold and Underline: | 1204 Substrate |
| Bold, Italics and Underline: | Linker 2 |
| Normal text: | anti-EGFR antibody |
| | derived sequence |

In some embodiments, the spacer sequence for the light chain 3954-1204-C225v5 activatable antibody can include an N-terminal variant, such as for example, a spacer selected from the group consisting of GQSGQ (SEQ ID NO: 235), QSGQ (SEQ ID NO: 236), SGQ (SEQ ID NO: 237), GQ and Q. In these embodiments, all other elements of the 3954-1204-C225v5 activatable antibodies, e.g., the heavy chain sequence, the light chain sequence, the 3954 mask, linker 1, the 1204 substrate, and linker 2, all remain the same as shown above in SEQ ID NO: 4.

The 3954-NSUB-C225v5 masked anti-EGFR antibody construct includes the same heavy chain as the 3954-1204-C225v5 activatable anti-EGFR antibody shown above. The 3954-NSUB-C225v5 masked anti-EGFR antibody construct includes the following light chain sequence:

3954-NSUB-C225v5 Masked Antibody Light
Chain Nucleotide Sequence:
[Spacer (SEQ ID NO: 5)][Mask (SEQ ID NO: 6)]
[Linker 1-Noncleavable Substrate-Linker 2 (SEQ ID
NO: 19)][C225 (SEQ ID NO: 10)]
(SEQ ID NO: 17)
[caaggccagtctggccag][tgcatctcacctcgtggttgtccggacggc
ccatacgtcatgtac][ggctcgagcggtggcagcggtggctctggtggct
caggtggaggctcgggcggtgggagcggcggttct][cagatcttgctgac
ccagagcccggtgattctgagcgtgagcccgggcgaacgtgtgagctttag
ctgccgcgcgagccagagcattggcaccaacattcattggtatcagcagcg
caccaacggcagcccgcgcctgctgattaaatatgcgagcgaaagcattag
cggcattccgagccgctttagcggcagcggcagcggcaccgattttaccct
gagcattaacagcgtggaaagcgaagatattgcggattattattgccagca
gaacaacaactggccgaccacctttggcgcgggcaccaaactggaactgaa
acgtacggtggctgcaccatctgtcttcatcttcccgccatctgatgagca
gttgaaatctggaactgcctctgttgtgtgcctgctgaataacttctatcc
cagagaggccaaagtacagtggaaggtggataacgccctccaatcgggtaa
ctcccaggagagtgtcacagagcaggacagcaaggacagcacctacagcct
cagcagcaccctgacgctgagcaaagcagactacgagaaacacaaagtcta
cgcctgcgaagtcacccatcagggcctgagctcgcccgtcacaaagagctt
caacaggggagagtgttag]

| | |
|---|---|
| Bold: | Spacer |
| Underline: | Mask |
| Italics and Underline: | Linker 1-Noncleavable |
| | substrate-Linker 2 |
| Normal text: | anti-EGFR antibody |
| | derived sequence |

3954-NSUB-C225v5 Masked Antibody Light
Chain Amino Acid Sequence:
[Spacer (SEQ ID NO: 11)][Mask (SEQ ID NO: 12)]
[Linker 1-Noncleavable Substrate-Linker 2 (SEQ ID
NO: 20)][C225 (SEQ ID NO: 16)]
(SEQ ID NO: 18)
[QGQSGQ][CISPRGCPDGPYVMY][GSSGGSGGSGGSGGGSGGGSGGS]
[QILLTQSPVILSVSPGERVSFSCRASQSIGTNIHWYQQRTNGSPRLLIK
YASESISGIPSRFSGSGSGTDFTLSINSVESEDIADYYCQQNNNWPTTFG
AGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC*]

| | |
|---|---|
| Bold: | Spacer |
| Underline: | Mask |
| Italics and Underline: | Linker 1-Noncleavable |
| | substrate-Linker 2 |
| Normal text: | anti-EGFR antibody |
| | derived sequence |

Reducing agent: The studies provided herein use the reducing agent TCEP (tris(2-carboxyethyl) phosphine).

Protocol for TCEP partial reduction of Anti-EGFR activatable antibody and subsequent conjugation to Maleimide Alexa-680: Bond-Breaker® TCEP Solution (neutral pH solution, Thermo Scientific) is used at various molar ratios of TCEP to an activatable antibody that, in the cleaved state (i.e., activated state), binds Epidermal Growth Factor Receptor, and the anti-EGFR activatable antibody is formulated in PBS. For example, the ratio of reducing agent, e.g., TCEP, to activatable antibody to be tested can include a ratio in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1. It is to be understood that while the examples provided herein use an anti-EGFR activatable antibody referred to herein as 3954-1204-C225v5, these methods are applicable to any activatable antibody having two or more cysteine residues, where it is desired that only a portion of the total number of cysteine residues in the activatable antibody be reduced prior to conjugation. This is referred to herein as “partial reduction.”

Briefly, a TCEP solution at twice the final concentration was mixed 1:1 (volume:volume) with 3954-1204-C225v5 to result in the final TCEP:(3954-1204-C225v5) ratio desired. The final solution was then incubated at 37° C. for specified periods of time for the reduction reaction to progress. At the end of the reduction reaction, the solution was cooled to room temperature and Maleimide Alexa-680 (Invitrogen) was added into the solution (Maleimide Alexa-680 was used at half of the reduction volume and at a concentration equal to 10× molar concentration of TCEP during the reduction reaction; for example, if the original reduction reaction comprised 50 microliters (ul) of 13.2 uM 3954-1204-C225v5 and 50 ul of 52.8 uM TCEP, then 50 ul 264 uM Maleimide Alexa-680 would be used) to begin the Alexa-680 conjugation. The conjugation reaction proceeded for 2 hours at room temperature in a light tight container. After the 2-hour reaction, the solution was spun down and buffer exchanged into PBS using a PD-10 column (GE Healthcare) or equivalent using manufacturer's instructions. The final conjugated product was analyzed using a UV Spectrophotometer to determine final protein concentration and the degree of labeling of the Alexa-680 dye.

Protocol for the analysis of Maleimide Alexa-680 conjugated Anti-EGFR activatable antibody using LabChip GXII: A HT Protein Express LabChip (Perkin Elmer) was prepared according to manufacturer's instructions using either the Pico Protein Express protocol or the HT Protein Express protocol: the Pico Protein Express protocol was used to analyze the Alexa-680 conjugated portion of the TCEP reduced 3954-1204-C225v5; the HT Protein Express protocol was used to analyze the total protein in the TCEP reduced, Alexa-680 conjugated 3954-1204-C225v5. TCEP reduced, Alexa-680 conjugated 3954-1204-C225v5 was prepared for the GXII analysis using Perkin Elmer's instructions. The sample was analyzed using the 200 series of the LabChip GXII analysis protocol (High sensitivity for the HT protocol and Pico for the Pico protocol). Resulting data was analyzed using the LabChip GXII software.

Protocol for EGFR binding ELISA: NUNC Maxisorp flatbottom 96 well plates were coated with 50 ul/well, 2 ug/ml human EGFR-Fc fusion protein (R&D Systems) in Hank's Balanced Salt Solution (HBSS, Teknova) for 2 hours at room temperature. At the end of the 2 hour coating, the liquid contents of the plate were evacuated and 250 ul/well of HBSS containing 1% BSA was introduced and allowed to block the plate for 30 minutes at room temperature. At the end of the blocking period, liquid contents of the 96-well plate were removed and serially diluted samples (i.e., 3954-1204-C225v5, Alexa-680 conjugated 3954-1204-C225v5, uPA-activated 3954-1204-C225v5, uPA-activated Alexa-680 conjugated 3954-1204-C225v5, and C225 (a cetuximab antibody), starting at a concentration of 100 ug/ml and diluted by a factor of 3 per dilution step) were introduced at 50 ul/well. The plate was incubated at room temperature for 1 hour. At the end of the hour, the plate was washed with HBSS containing 0.05% Tween-20 using a BioTek ELx450 Select CW plate washer (300 ul/well wash volume, 6 cycles of aspiration and wash). Washed plates were tapped dry and 50 ul/well of 400 ng/ml Horse Radish Peroxidase conjugated Goat anti-Human IgG Fab′2 specific antibody (Jackson ImmunoResearch) were introduced and incubated for 30 minutes at room temperature. The plates were washed as previously stated and 100 ul/well of 1-Step TMB Substrate (Thermo Scientific) was introduced. Color change was observed and the reaction was stopped by the addition of 100 ul/well of 1 M HCl (Fisher Scientific). The reacted plate was analyzed using a BioTek EL800 plate reader at O.D. 450. Data were computed using Excel (Microsoft) and the result was plotted using Prism 6 (GraphPad).

### Example 2. TCEP-Mediated Reduction of Activatable Antibodies

The compositions and methods provided herein determine the combination of reagents and reaction conditions that produce the desired partial reduction followed by conjugation. When reduction and subsequent conjugation is not controlled properly, activatable antibodies will be completely reduced, and the masking efficiency of the activatable antibody is compromised. For example, when the reducing agent is used at a ratio of 20:1 (reducing agent to activatable antibody), the activatable antibody was completely reduced into free heavy chain and free light chain. Attempts to produce a milder reduction (i.e., less than complete reduction), for example, by immobilizing a reducing agent, were too mild and did not sufficiently reduce the activatable antibody to allow for subsequent conjugation. In these studies, bands corresponding to predominantly intact IgG (high molecular weight band >150 kDa) were observed in all reduction conditions.

Studies were conducted to determine the range of reducing agent to activatable antibody. At lower ratios, for example, in the range of 0.5:1 to 2:1 (reducing agent to activatable antibody), some reduction was achieved, and the activatable antibody integrity and masking efficiency were retained. At ratios of 1.5:1 to 5:1 (reducing agent to activatable antibody), reduction time from 30 minutes to 2 hours, there was an increasing amount of reduced activatable antibody species corresponding to the molecular weight of one heavy chain and one light chain activatable antibody. The partially reduced activatable antibody maintained the EGFR binding characteristics of the original non-reduced and masked activatable antibody demonstrating that the activatable antibody partially reduced under these conditions was capable of maintaining the original masking efficiency. At the identified ratio of reducing agent to activatable antibody and reduction time, an inter-chain disulfide-reduced activatable antibody can be produced to allow for subsequent maximum conjugation through free cysteines while maintaining the masking efficiency of the original, non-reduced activatable antibody.

At a reduction time of 2 hours, a ratio of reducing agent to activatable antibody ratio above 5:1 was too reductive to maintain the original masking efficiency of the tested activatable antibodies. The varied shift in masking efficiency loss and the varied amounts of partially reduced activatable antibody subspecies showed that the tested activatable antibodies have different tolerance to reducing agent-mediated reduction, for example, TCEP-mediated reduction. The varied combination of antibody, linkers, cleavable moiety (CM) and masking moiety (MM) results in a spectrum in the tolerance of the activatable antibody for reducing agent-mediated reduction, for example, TCEP-mediated reduction.

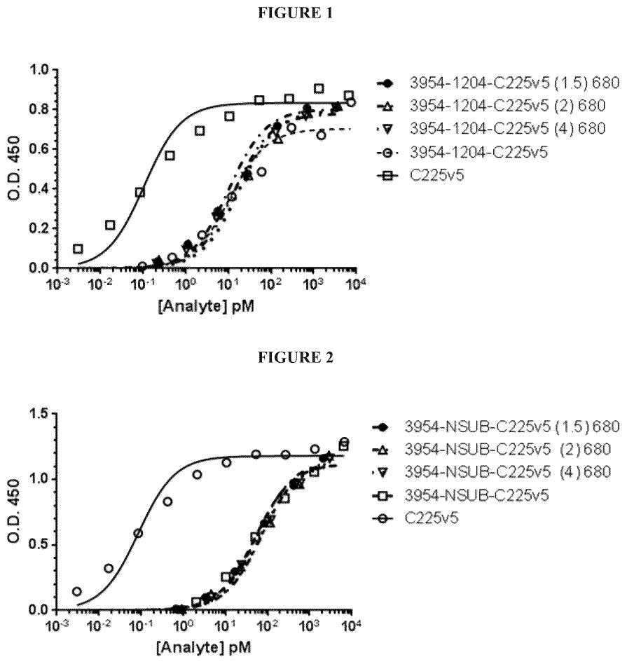

In one set of studies described herein, an activatable anti-EGFR antibody referred to as 3954-1204-C225v5 was reduced at various ratios of TCEP to activatable antibody (e.g., from about 1.5:1 to about 4:1) using a 90-minute reduction time. In some instances, reduction was followed by conjugation to a fluorescent dye, Alexa 680. The results of these studies (at TCEP to activatable antibody ratios of 1.5:1, 2:1, and 4:1) are shown in FIG. 1.

In another set of studies described herein, an anti-EGFR antibody construct that includes an antigen-binding portion that specifically binds EGFR, a masking moiety, and a non-cleavable linker (referred to as 3954-NSUB-C225v5) was reduced at various ratios of TCEP to activatable antibody (e.g., from about 1.5:1 to about 4:1) using a 90-minute reduction time. In some instances, reduction was followed by conjugation to Alexa 680. The results of these studies (at TCEP to activatable antibody ratios of 1.5:1, 2:1, and 4:1) are shown in FIG. 2.

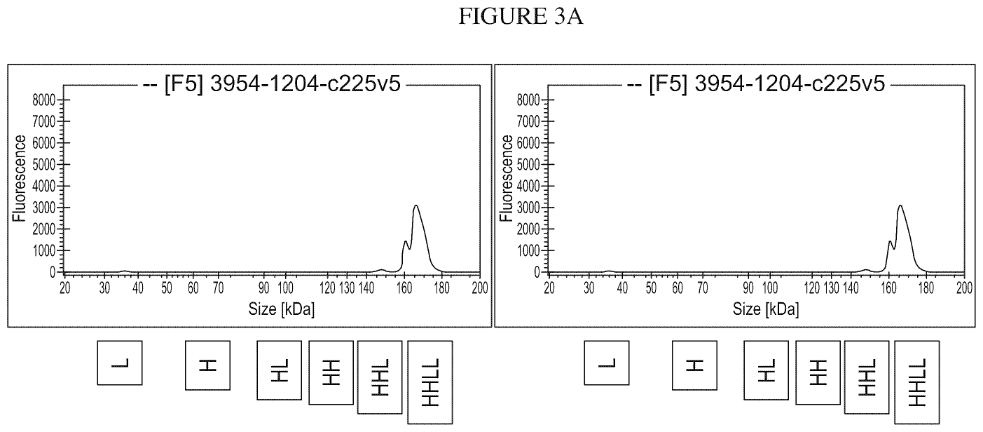

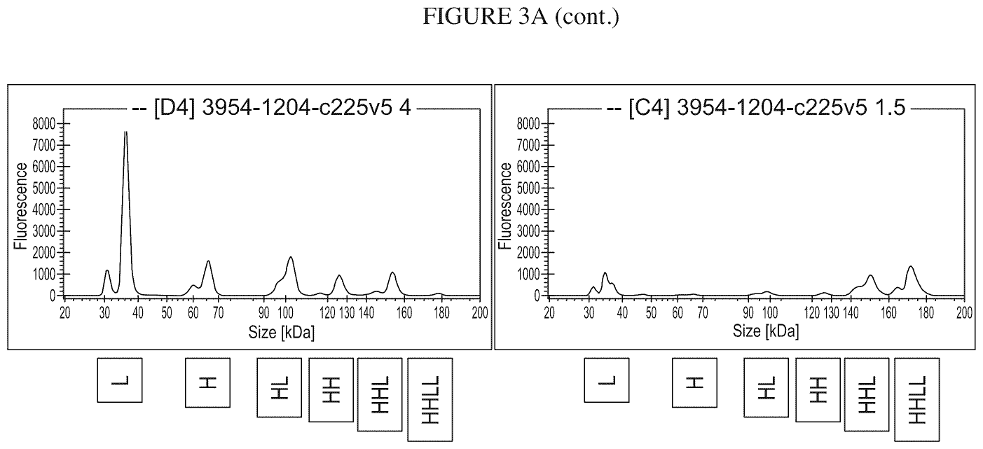

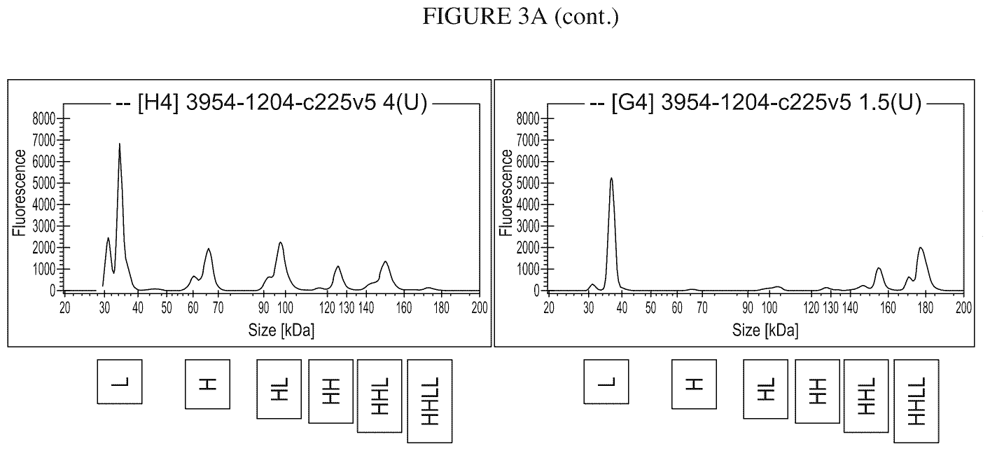

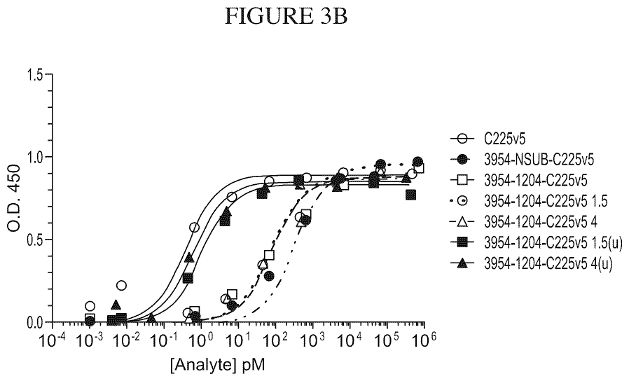

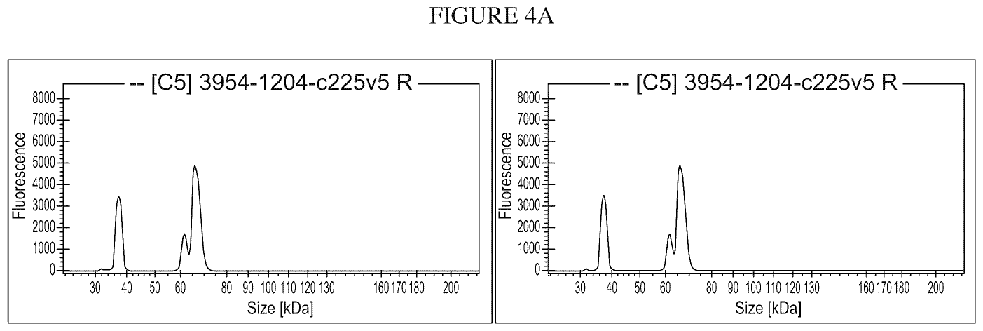

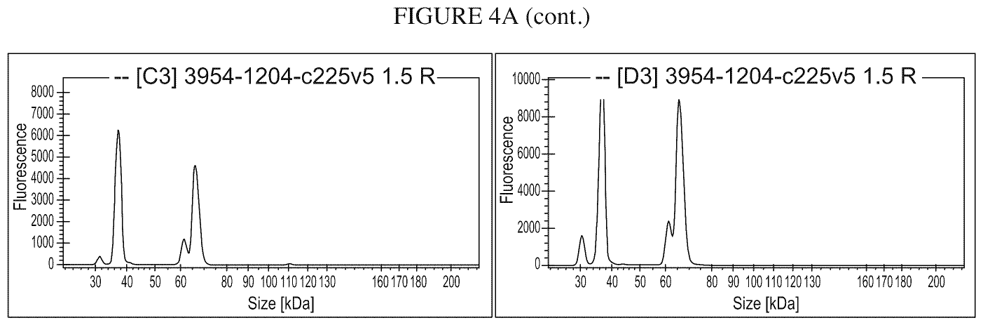

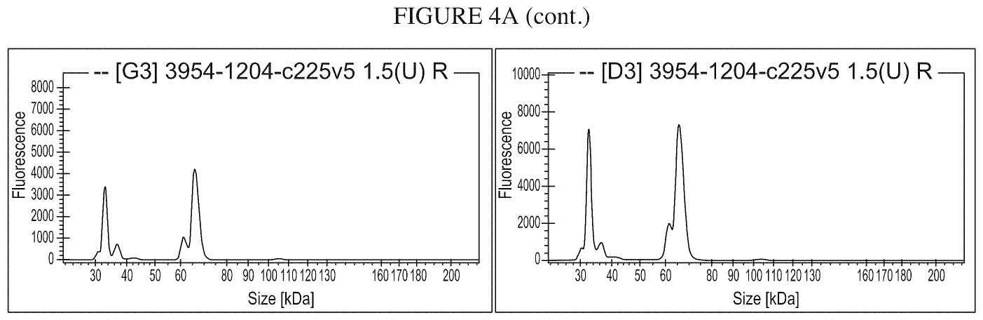

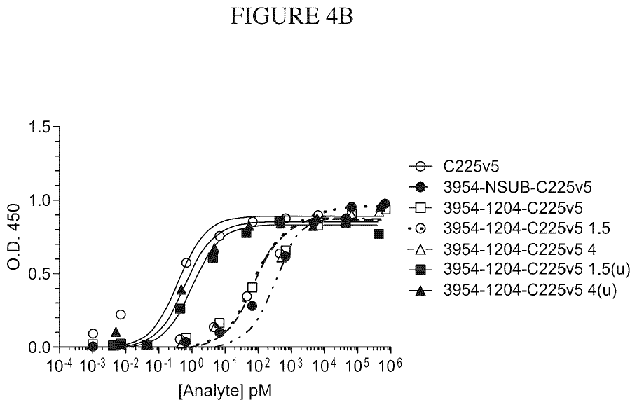

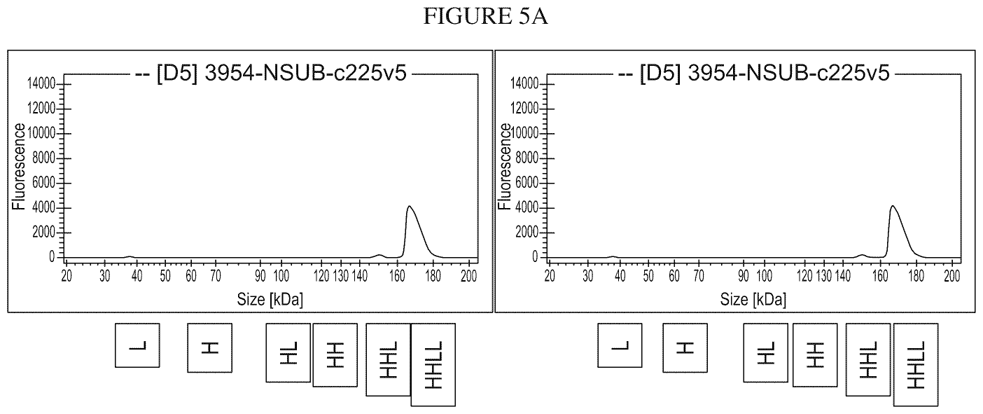

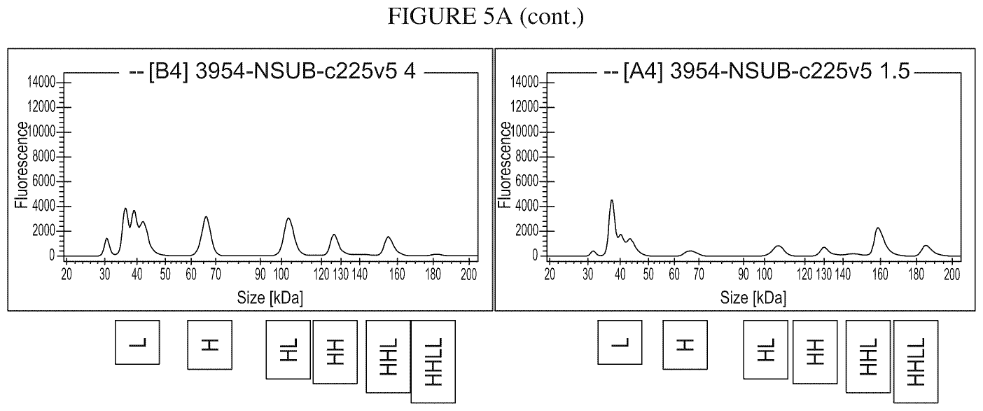

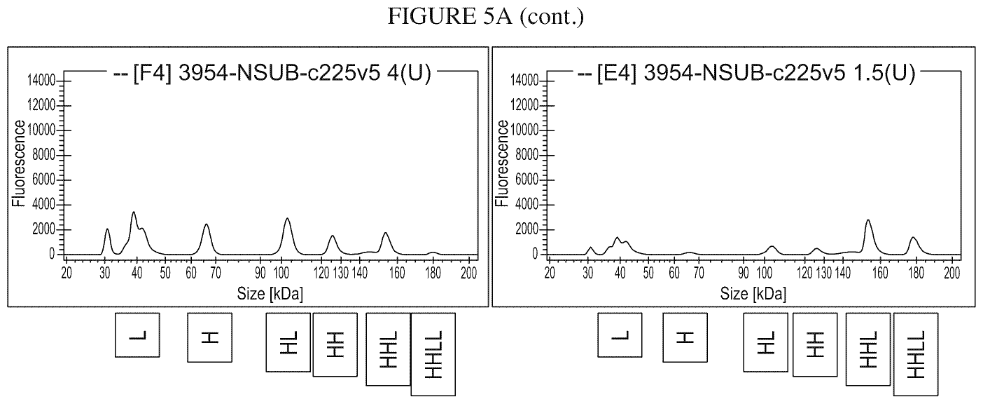

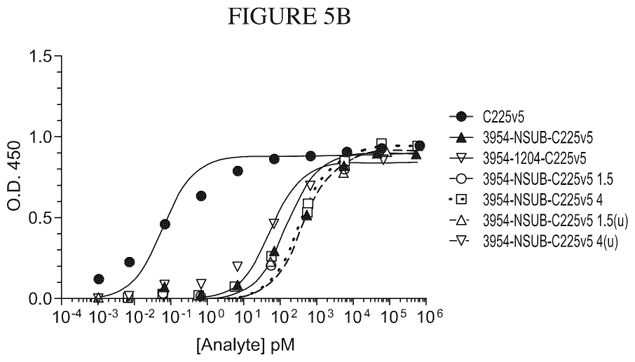

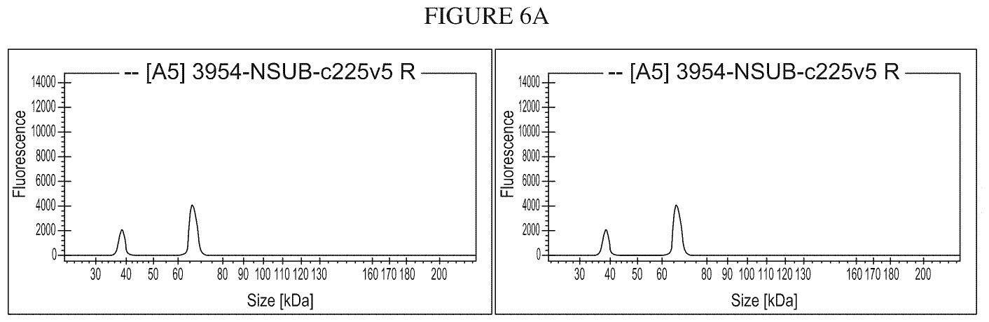

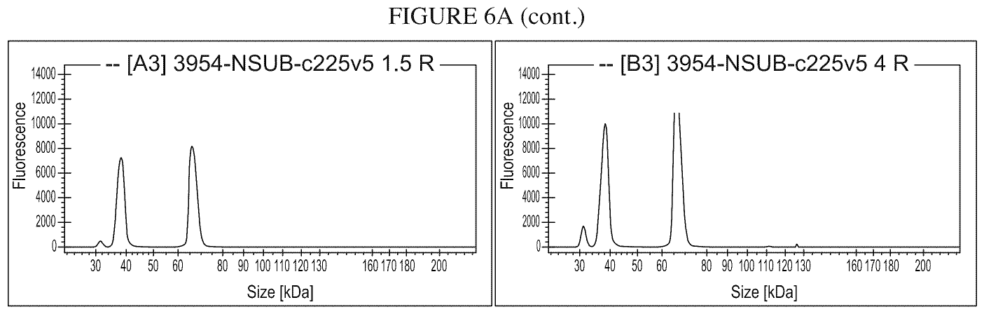

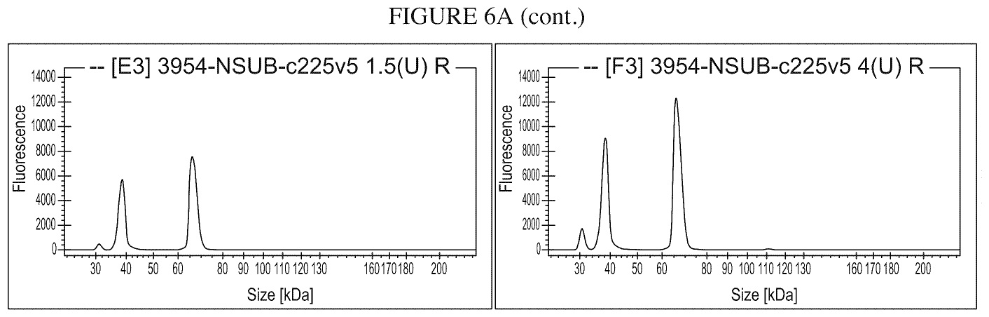

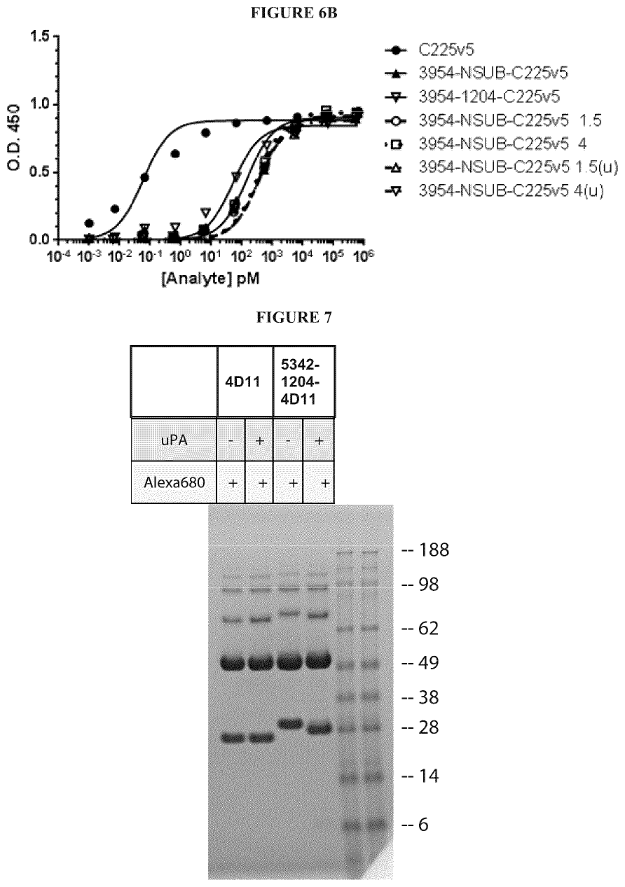

Using thiol conjugatable Alexa 680 as a surrogate for thiol conjugatable toxin, these studies demonstrate varying degrees of Alexa 680 conjugation dependent on both TCEP to activatable antibody ratio and time of reduction. The conjugation of thiol conjugatable Alexa 680 to TCEP-partially reduced 3954-1204-C225v5 or 3954-NSUB-C225v5 does not significantly change the titration profile of 3954-1204-C225v5 or 3954-NSUB-C225v5 to EGFR. Thus, partial reduction and subsequent thiol conjugation of Alexa 680 can be done in such a way as to maintain the masking efficiency of activatable antibodies. FIGS. 3 through 6 demonstrate that partial reduction and subsequent thiol conjugation of Alexa 680 Fluor® can be done in such a way as to also maintain activation of 3954-1204-C225v5 by uPA and not to lead to activation of 3954-NSUB-C225v5.

Further studies indicated that a degree of labeling (molar ratio of Alexa 680 Fluor® vs. activatable antibody) of 3.8 was achieved for 3954-1204-C225v5 and a degree of labeling of 3.5 was achieved for 3954-NSUB-C225v5.

### Example 3. Mass Spectrometry Analysis of Conjugated Activatable Antibodies

Molecular weight determination by MALDI Mass Spectrometry (MALDI MS): In MALDI MS, the dissolved sample is deposited on a metal target and the peptides and proteins are co-crystallized with a light-absorbing matrix. A laser beam is directed at the dry matrix sample, the sample molecules are desorbed and ionized and the masses are measured in a time-of-flight (TOF) mass analyzer. Proteins are observed in the mass spectrum (mass-over-charge spectrum m/z) as singly (m/z MH+) as well as multiple charged ions.

In the present analysis, the partially reduced and conjugated activatable antibodies (intact and DTT-reduced) were purified using C4 ziptips from Millipore. Each purified sample was mixed with 2,5-dihydroxyacetophononc/diammonium hydrogen citrate (DHAP/DAHC) matrix and spotted onto a Big Anchor target from Bruker. Mass spectra were obtained on an Autoflex Speed MALDI TOF/TOF mass spectrometer in linear mode using Compas 1.4 control and processing software. The mass spectra were calibrated by external quadratic calibration using Bruker Protein Calibration Standard 1 or 2. The sample mass was calculated from the least charged ion within the calibrated range of the mass spectrum.

Each Alexa-680 molecule added approximately 1000 dalton (˜1 kDa) of mass to the activatable antibody. A comparison of the molecular weight of the unconjugated 3954-1204-C225v5 to the conjugated 3954-1204-C225v5 enables estimation of the number of Alexa-680® molecules that has been conjugated upon 3954-1204-C225v5.

The MALDI-MW data indicated that up to four 1-kDa molecules were attached to the activatable anti-EGFR antibody 3954-1204-C225v5. From the reduced samples, it was determined that this modification was most likely heterogeneous, as the light chain was observed with 0 and 1 modification(s) and the heavy chain was observed with 0, 1 or 2 modifications.

It was determined from the MALDI-MS data on digested samples that all peptides containing cysteine residues were still observed in the conjugated sample (Cyt04-680 high). The MALDI-MW data also confirmed that the antibody was not fully labeled by Alexa-680®.

### Example 4. Materials and Methods

The examples provided herein use an anti-Jagged activatable antibody referred to herein as activatable antibody 5342-1204-4D11 (also referred to herein as 5342-1204-4D11 activatable antibody or 5342-1204-4D11) that includes a Jagged-binding sequence, a masking moiety (MM), and a cleavable moiety (CM) that is a substrate for a protease. It is to be understood that while the examples provided herein use these anti-Jagged activatable antibody constructs, these methods are applicable to any activatable antibody having two or more cysteine residues, where it is desired that only a portion of the total number of cysteine residues in the activatable antibody be reduced prior to conjugation. This is referred to herein as “partial reduction.”

It should be further understood that the examples provided herein use a fluorescent agent, Alexa-680 Fluor® (also referred to herein as Alexa 680®), as the agent that is to be conjugated to an activatable antibody. This particular dye was chosen because it has a molecular weight that is similar to a known cytotoxic agent, MMAE. However, this fluorescent agent is merely used as an example, and the compositions and methods used herein are useful with any number of conjugated agents, including by way of non-limiting example, toxins and other payload agents. The compositions and methods are not limited to agents of any particular molecular weight, size or other such characteristic.

Anti-Jagged activatable antibody constructs: The 5342-1204-4D11 activatable anti-Jagged antibody construct includes the following heavy and light chain sequences:

5342-1204-4D11 Activatable Antibody Heavy
Chain Nucleotide Sequence:
(SEQ ID NO: 231)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTGTCAAGT
ATTGACCCGGAAGGTCGGCAGACATATTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTATATTACTGTGCGAAAGACATC
GGCGGCAGGTCGGCCTTTGACTACTGGGGCCAGGGAACCCTGGTCACCGT
CTCCTCAGCTAGCACCAAGGGCCCATCGGTCTTCCCCCTGGCACCCTCCT
CCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGT
TGAGCCCAAATCTTGTGACAAAACTCACACATGCCCACCGTGCCCAGCAC
CTGAACTCCTGGGGGGACCGTCAGTCTTCCTCTTCCCCCCAAAACCCAAG
GACACCCTCATGATCTCCCGGACCCCTGAGGTCACATGCGTGGTGGTGGA
CGTGAGCCACGAAGACCCTGAGGTCAAGTTCAACTGGTACGTGGACGGCG
TGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTG
TACACCCTGCCCCCATCCCGGGAGGAGATGACCAAGAACCAGGTCAGCCT
GACCTGCCTGGTCAAAGGCTTCTATCCCAGCGACATCGCCGTGGAGTGGG
AGAGCAATGGGCAGCCGGAGAACAACTACAAGACCACGCCTCCCGTGCTG
GACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTC
TGCACAACCACTACACGCAGAAGAGCCTCTCCCTGTCTCCGGGTAAA
5342-1204-4D11 Activatable Antibody Heavy
Chain Amino Acid Sequence:
(SEQ ID NO: 245)
EVQLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
5342-1204-4D11 Activatable Antibody Light
Chain Nucleotide Sequence:
(SEQ ID NO: 233)
CAAGGCCAGTCTGGCCAGTGCAATATTTGGCTCGTAGGTGGTGATTGCAG
GGGCTGGCAGGGGGGCTCGAGCGGTGGCAGCGGTGGCTCTGGTGGTCTGA
GCGGCCGTTCCGATAATCATGGCGGCGGTTCTGACATCCAGATGACCCAG
TCTCCATCCTCCCTGTCTGCATCTGTAGGAGACAGAGTCACCATCACTTG
CCGGGCAAGTCAGAGCATTAGCAGCTATTTAAATTGGTATCAGCAGAAAC
CAGGGAAAGCCCCTAAGCTCCTGATCTATGCGGCATCCAGTTTGCAAAGT
GGGGTCCCATCAAGGTTCAGTGGCAGTGGATCTGGGACAGATTTCACTCT
CACCATCAGCAGTCTGCAACCTGAAGATTTTGCAACTTACTACTGTCAAC
AGACGGTTGTGGCGCCTCCGTTATTCGGCCAAGGGACCAAGGTGGAAATC
AAACGTACGGTGGCTGCACCATCTGTCTTCATCTTCCCGCCATCTGATGA
GCAGTTGAAATCTGGAACTGCCTCTGTTGTGTGCCTGCTGAATAACTTCT
ATCCCAGAGAGGCCAAAGTACAGTGGAAGGTGGATAACGCCCTCCAATCG
GGTAACTCCCAGGAGAGTGTCACAGAGCAGGACAGCAAGGACAGCACCTA
CAGCCTCAGCAGCACCCTGACGCTGAGCAAAGCAGACTACGAGAAACACA
AAGTCTACGCCTGCGAAGTCACCCATCAGGGCCTGAGCTCGCCCGTCACA
AAGAGCTTCAACAGGGGAGAGTGT
5342-1204-4D11 Activatable Antibody Light
Chain Amino Acid Sequence:
(SEQ ID NO: 234)
QGQSGQCNIWLVGGDCRGWQGGSSGGSGGSGGLSGRSDNHGGGSDIQMTQ
SPSSLSASVGDRVTITCRASQSISSYLNWYQQKPGKAPKLLIYAASSLQS
GVPSRFSGSGSGTDFTLTISSLQPEDFATYYCQQTVVAPPLFGQGTKVEI
KRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVT
KSFNRGEC

In some embodiments, the spacer sequence for the light chain 5342-1204-4D11 activatable antibody can include an N-terminal variant, such as for example, a spacer selected from the group consisting of GQSGQ (SEQ ID NO: 235), QSGQ (SEQ ID NO: 236), SGQ (SEQ ID NO: 237), GQ and Q. In these embodiments, all other elements of the 5342-1204-4D11 activatable antibodies, e.g., the heavy chain sequence, the light chain sequence, the 5342 mask, linker 1, the 1204 substrate, and linker 2, all remain the same as shown above in SEQ ID NO: 234.

Reducing agent: The studies provided herein use the reducing agent TCEP (tris(2-carboxyethyl) phosphine).

Protocol for TCEP partial reduction of Anti-Jagged activatable antibody and subsequent conjugation to Maleimide Alexa-680: Bond-Breaker® TCEP Solution (neutral pH solution, Thermo Scientific) is used at various molar ratios of TCEP to an activatable antibody that, in the cleaved state (i.e., activated state), binds Epidermal Growth Factor Receptor, and the anti-Jagged activatable antibody is formulated in PBS. For example, the ratio of reducing agent, e.g., TCEP, to activatable antibody to be tested can include a ratio in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1. It is to be understood that while the examples provided herein use an anti-Jagged activatable antibody referred to herein as 5342-1204-4D11, these methods are applicable to any activatable antibody having two or more cysteine residues, where it is desired that only a portion of the total number of cysteine residues in the activatable antibody be reduced prior to conjugation. This is referred to herein as “partial reduction.”

Briefly, a TCEP solution at twice the final concentration was mixed 1:1 (volume:volume) with 5342-1204-4D11 to result in the final TCEP:(5342-1204-4D11) ratio desired. The final solution was then incubated at 37° C. for specified periods of time for the reduction reaction to progress. At the end of the reduction reaction, the solution was cooled to room temperature and Maleimide Alexa-680 (Invitrogen) was added into the solution (Maleimide Alexa-680 was used at half of the reduction volume and at a concentration equal to 10× molar concentration of TCEP during the reduction reaction; for example, if the original reduction reaction comprised 50 microliters (ul) of 13.2 uM 5342-1204-4D11 and 50 ul of 52.8 uM TCEP, then 50 ul 264 uM Maleimide Alexa-680 would be used) to begin the Alexa-680 conjugation. The conjugation reaction proceeded for 2 hours at room temperature in a light tight container. After the 2-hour reaction, the solution was spun down and buffer exchanged into PBS using a PD-10 column (GE Healthcare) or equivalent using manufacturer's instructions. The final conjugated product was analyzed using a UV Spectrophotometer to determine final protein concentration and the degree of labeling of the Alexa-680 dye.

Protocol for the analysis of Maleimide Alexa-680 conjugated Anti-Jagged activatable antibody using LabChip GXII: A HT Protein Express LabChip (Perkin Elmer) was prepared according to manufacturer's instructions using either the Pico Protein Express protocol or the HT Protein Express protocol: the Pico Protein Express protocol was used to analyze the Alexa-680 conjugated portion of the TCEP reduced 5342-1204-4D11; the HT Protein Express protocol was used to analyze the total protein in the TCEP reduced, Alexa-680 conjugated 5342-1204-4D11. TCEP reduced, Alexa-680 conjugated 5342-1204-4D11 was prepared for the GXII analysis using Perkin Elmer's instructions. The sample was analyzed using the 200 series of the LabChip GXII analysis protocol (High sensitivity for the HT protocol and Pico for the Pico protocol). Resulting data was analyzed using the LabChip GXII software.

Protocol for Jagged binding ELISA: NUNC Maxisorp flatbottom 96 well plates were coated with 50 ul/well, 2 μg/ml human Jagged-Fc fusion protein (R&D Systems) in Hank's Balanced Salt Solution (HBSS, Teknova) for 2 hours at room temperature. At the end of the 2 hour coating, the liquid contents of the plate were evacuated and 250 ul/well of HBSS containing 1% BSA was introduced and allowed to block the plate for 30 minutes at room temperature. At the end of the blocking period, liquid contents of the 96-well plate were removed and serially diluted samples (i.e., 5342-1204-4D11, Alexa-680 conjugated 5342-1204-4D11, uPA-activated 5342-1204-4D11, uPA-activated Alexa-680 conjugated 5342-1204-4D11, and 4D11 (an anti-Jagged antibody), starting at a concentration of 100 μg/ml and diluted by a factor of 3 per dilution step) were introduced at 50 ul/well. The plate was incubated at room temperature for 1 hour. At the end of the hour, the plate was washed with HBSS containing 0.05% Tween-20 using a BioTek ELx450 Select CW plate washer (300 ul/well wash volume, 6 cycles of aspiration and wash). Washed plates were tapped dry and 50 ul/well of 400 ng/ml Horse Radish Peroxidase conjugated Goat anti-Human IgG Fab′2 specific antibody (Jackson ImmunoResearch) were introduced and incubated for 30 minutes at room temperature. The plates were washed as previously stated and 100 ul/well of 1-Step TMB Substrate (Thermo Scientific) was introduced. Color change was observed and the reaction was stopped by the addition of 100 ul/well of 1 M HCl (Fisher Scientific). The reacted plate was analyzed using a BioTek EL800 plate reader at O.D. 450. Data were computed using Excel (Microsoft) and the result was plotted using Prism 6 (GraphPad).

### Example 5. TCEP-Mediated Reduction of Activatable Antibodies

The compositions and methods provided herein determine the combination of reagents and reaction conditions that produce the desired partial reduction followed by conjugation. When reduction and subsequent conjugation is not controlled properly, activatable antibodies will be completely reduced, and the masking efficiency of the activatable antibody is compromised.

Studies were conducted to determine the range of reducing agent to activatable antibody. At lower ratios, for example, in the range of 0.5:1 to 2:1 (reducing agent to activatable antibody), some reduction was achieved, and the activatable antibody integrity and masking efficiency were retained. At ratios of 1.5:1 to 5:1 (reducing agent to activatable antibody), reduction time from 30 minutes to 2 hours, there was an increasing amount of reduced activatable antibody species corresponding to the molecular weight of one heavy chain and one light chain activatable antibody. The partially reduced activatable antibody maintained the Jagged binding characteristics of the original non-reduced and masked activatable antibody demonstrating that the activatable antibody partially reduced under these conditions was capable of maintaining the original masking efficiency. At the identified ratio of reducing agent to activatable antibody and reduction time, an inter-chain disulfide-reduced activatable antibody can be produced to allow for subsequent maximum conjugation through free cysteines while maintaining the masking efficiency of the original, non-reduced activatable antibody.

In one set of studies described herein, an activatable anti-Jagged antibody referred to as 5342-1204-4D11 was reduced at a ratio of TCEP to activatable antibody equaling 4; 1 using a 120-minute reduction time. In some instances, reduction was followed by conjugation to a fluorescent dye, Alexa 680. The results of these studies (at TCEP to activatable antibody ratios of 4:1) are shown in FIG. 7.

Using thiol conjugatable Alexa 680 as a surrogate for thiol conjugatable toxin, these studies demonstrate varying degrees of Alexa 680 conjugation dependent on both TCEP to activatable antibody ratio and time of reduction. The conjugation of thiol conjugatable Alexa 680 to TCEP-partially reduced 5342-1204-4D11 does not significantly change the titration profile of 5342-1204-4D11 to Jagged. Thus, partial reduction and subsequent thiol conjugation of Alexa 680 can be done in such a way as to maintain the masking efficiency of activatable antibodies. FIG. 8 demonstrates that partial reduction and subsequent thiol conjugation of Alexa 680 Fluor® can be done in such a way as to also maintain activation of 5342-1204-4D11 by uPA.

### Example 6. Materials and Methods

Antibodies and activatable antibodies: The examples provided herein use an anti-Jagged activatable antibody, referred to herein as activatable antibody 5342-1204-4D11, which is described herein. It is to be understood that while the examples provided herein use these anti-Jagged activatable antibody constructs, these methods are applicable to any activatable antibody having two or more cysteine residues, where it is desired that only a portion of the total number of cysteine residues in the activatable antibody be reduced prior to conjugation. This is referred to herein as “partial reduction.”

The examples provided herein also use an anti-Jagged antibody, referred to herein as anti-Jagged antibody 4D11 (also referred to as antibody 4D11 and 4D11 antibody). The antibody 4D11 includes the following heavy and light chain sequences:

4D11 Antibody Heavy Chain Nucleotide Sequence:
(SEQ ID NO: 231)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTGTCAAGT
ATTGACCCGGAAGGTCGGCAGACATATTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTATATTACTGTGCGAAAGACATC
GGCGGCAGGTCGGCCTTTGACTACTGGGGCCAGGGAACCCTGGTCACCGT
CTCCTCAGCTAGCACCAAGGGCCCATCGGTCTTCCCCCTGGCACCCTCCT
CCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAG
CGGCGTGCACACCTTCCCGGCTGTCCTACAGTCCTCAGGACTCTACTCCC
TCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGT
TGAGCCCAAATCTTGTGACAAAACTCACACATGCCCACCGTGCCCAGCAC
CTGAACTCCTGGGGGGACCGTCAGTCTTCCTCTTCCCCCCAAAACCCAAG
GACACCCTCATGATCTCCCGGACCCCTGAGGTCACATGCGTGGTGGTGGA
CGTGAGCCACGAAGACCCTGAGGTCAAGTTCAACTGGTACGTGGACGGCG
TGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTG
TACACCCTGCCCCCATCCCGGGAGGAGATGACCAAGAACCAGGTCAGCCT
GACCTGCCTGGTCAAAGGCTTCTATCCCAGCGACATCGCCGTGGAGTGGG
AGAGCAATGGGCAGCCGGAGAACAACTACAAGACCACGCCTCCCGTGCTG
GACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTC
TGCACAACCACTACACGCAGAAGAGCCTCTCCCTGTCTCCGGGTAAA
4D11 Antibody Heavy Chain Amino Acid Sequence:
(SEQ ID NO: 245)
EVQLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
4D11 Antibody Light Chain Nucleotide Sequence:
(nucleotides 133-774 of SEQ ID NO: 233)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATGCG
GCATCCAGTTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGACGGTTGTGGCGCCTCCGTTATTCGGCCAA
GGGACCAAGGTGGAAATCAAACGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
4D11 Antibody Light Chain Amino Acid Sequence:
(SEQ ID NO: 244)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Additional antibodies used herein include Synagis® (palivizumab) and rituximab, both of which were purchased from Drug Products Service Labs (UCSF) and used as human IgG1 isotype controls.

SDS gel: Five to ten micrograms (ug) of sample were diluted in 7.5 microliters (ul) of water, final volume. 2.5 ul of 4× sample loading buffer (Invitrogen) with or without 1 ul of 10× reducing agent (Invitrogen) were added and the samples heated at ˜90° C. for ˜10 min in a heating block. Samples were loaded onto a 10% Bis-Tris (Novex) gel and run in MOPS buffer at 200 mV for ˜40 minutes. Gels were stained in Instant Blue (Expedion) for ˜1 hr followed by de-staining in multiple washes of water. Gel images were captured on the Imagequant.

Cell line: BxPC3 cells (ATCC CRL 1687) were maintained in CM (RPMI 1640 supplemented with 10% heat-inactivated fetal bovine serum (FBS). Prior to FACS binding cell dissociation buffer (Sigma #C5914) was used to dissociate the adherent cells.

Protocol for FACS cell binding: The binding of antibodies, activatable antibodies, or their immunoconjugate derivatives on BxPC3 cells was evaluated by an indirect immunofluorescence assay. Dissociated cells (50,000-100,000 per well) were pelleted in a 96-well v-bottom plate and incubated at 4° C. for 45 minutes with serial dilutions of test article in 50 to 100 ul of FACS buffer (FB; HBSS supplemented with 2% FBS). Control wells included human IgG1 isotype control (Synagis or rituximab) and no IgG containing wells. Cells were washed twice in cold FB and stained with Alexa 647 conjugated goat anti-human IgG (AF-647 conjugated affinity pure F(ab′)₂ fragment goat anti human IgG Fc gamma (γ) fragment specific, Jackson labs, #1909-606-170) for 30 minutes at 4° C. Cells were washed as before, fixed in 1% paraformaldehyde/FB and analyzed using a FACSAria flow cytometer (BD Biosciences). FCS files were analyzed in FCS Express (DeNovo) and mean fluorescence intensity (MFI) against dose titration plotted in GraphPad PRISM.

Protocol for in vitro cytotoxicity assay: Cells (4,000 per well) were plated in white walled 96-well plates in 50 ul CM. Cells were treated with an equal volume of serial dilutions of test article for 3 to 5 days. An equal volume (100 ul) of Cell Titer Glo reagent (Promega) was added to each well, according to manufacturer's instructions, and relative luminescence units (RLU) was measured on the Tecan Infinite M200 Pro. RLU against dose titration was plotted and curve fitting generated in GraphPad PRISM.

### Example 7. Production of Activatable Antibody Conjugates by TCEP-Mediated Partial Reduction and Conjugation

This example describes the use of partial reduction and conjugation methods of the invention to produce antibody conjugates and activatable antibody conjugates of the invention. It is to be understood that while the examples provided herein use anti-Jagged activatable antibody constructs, these methods are applicable to any activatable antibody having two or more cysteine residues, where it is desired that only a portion of the total number of cysteine residues in the activatable antibody be reduced prior to conjugation. This is referred to herein as “partial reduction.” In addition, these methods are applicable to any cleavable or non-cleavable linker and agent combination.

This example presents conjugates that display a maleimide PEG-valine-citrulline-para-aminobenzyloxycarbonyl-monomethyl auristatin D linker payload, referred to herein as “vc-MMAD,” or a maleimide PEG-valine-citrulline-para-aminobenzyloxycarbonyl-monomethyl auristatin E linker payload, referred to herein as “vc-MMAE.”

Conjugates of anti-Jagged activatable antibody 5342-1204-4D11 comprising the malcimide caproyl-valine-citruline monomethyl auristatin D linker payload (vc-MMAD) or the malcimide caproyl-valine-citruline monomethyl auristatin E linker payload (vc-MMAE), referred to herein as activatable antibody conjugate 5342-1204-4D11-vc-MMAD and activatable antibody conjugate 5342-1204-4D11-vc-MMAE, respectively, were prepared as follows.

The vc-MMAD and vc-MMAE reagents were prepared at Bayside Chemicals (Burlingame, CA). N,N, Dimethylacetamide (DMA), tris(2-carboxyethyl) phosphine (TCEP; cat #646547), and IN NaOH were purchased from Sigma. Prior to performing a conjugation, the following stock solutions were prepared: 5.0 mg/mL antibody or activatable antibody in PBS (total phosphate=4.25 mM), 5.0 mg/mL vc-MMAD or vc-MMAE in DMA, 1.0 mM TCEP in water, and 10 mM NaOH in water. TCEP was used at a range from 1:1 to 8:1 ratio, typically at a 2.5:1 equimolar ratio, of TCEP to antibody or activatable antibody.

A typical partial reduction and conjugation was performed as follows, wherein equivalents (eq) are reported relative to the antibody or activatable antibody. To 200 μL of antibody or activatable antibody in PBS in a tube was added 2.5 eq TCEP. The tube was closed and swirled, to generate a homogeneous solution, which was allowed to stand for 90 minutes at room temperature. The tube was opened, and 13 cq NaOH and 6.0 eq vc-MMAD or vc-MMAE were added. The tube was closed and swirled, to generate a homogeneous solution, which was allowed to stand at room temperature for 120 minutes. The tube was opened, and the reaction mixture was passed through a Zeba de-salting column (Thermo Scientific). The protein concentration in the filtrate was analyzed by UV spectrophotometry, and the product was analyzed by SDS gel and HIC-HPLC, and then tested for cell-binding and cell-killing activities. For the cell-binding and cell-killing assays, activatable antibody conjugates were activated by incubating a 0.5-mg sample of the activatable antibody conjugate in a 10% solution of recombinant human uPA protease (R and D systems MN-207-16) at 37° C. for 16 hr. Protease was removed from the thus activated activatable antibody conjugate by running the sample through a pre-washed MabSelect (GE Healthcare) column and eluting with 0.1 M glycine, followed by neutralization using Tris-HCl. In other conjugations, the TCEP eq varied from 1.0 to 8.0, and/or the NaOH eq varied from 0 to 13, and/or the reaction times after the addition of TCEP, or the additions of NaOH and vc-MMAD or vc-MMAD, varied from 60 min to 16 hours.

### Example 8. Use of Activatable Antibody Conjugates that Include Microtubule Inhibitor Monomethyl Auristatin D (MMAD)

This example demonstrates that activatable antibody conjugates comprising microtubule inhibitor MMAD display potent in vitro killing activity. The example also demonstrates that addition of the linker payload to the activatable antibody does not interfere with the ability of the masking moiety to block binding of the activatable antibody comprising such masking moiety to its target.

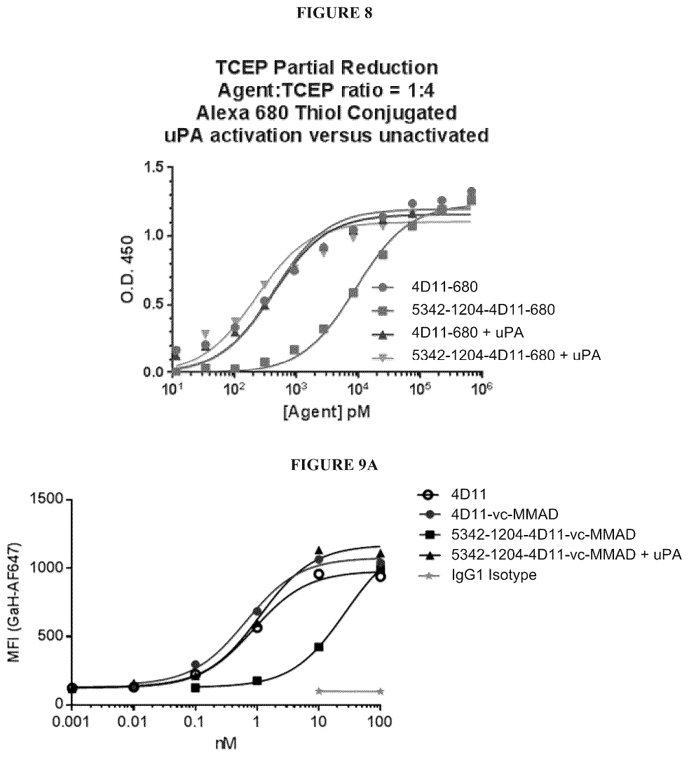

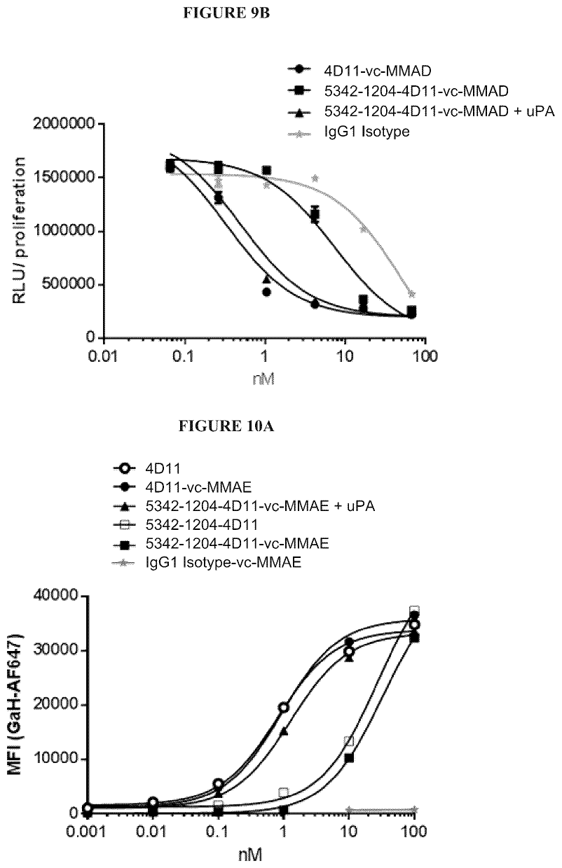

FIG. 9A demonstrates that partial reduction and subsequent thiol conjugation of anti-Jagged activatable antibody 5342-1204-4D11 or anti-Jagged antibody 4D11 with the vc-MMAD linker payload did not affect the binding behavior of the resultant conjugates to BxPC3 cells: There was a 38-fold reduction in IC50 of binding of activatable antibody conjugate 5342-1204-4D11-vc-MMAD (27 nM) compared to antibody conjugate 4D11-vc-MMAD (0.7 nM). Upon activation with uPA, binding of activated activatable antibody conjugate 5342-1204-4D11-vc-MMAD was comparable to that of antibody conjugate 4D11-vc-MMAD. The figure also demonstrates that Synagis (a human IgG1 isotype control) did not bind to BxPC3 cells.

Conjugation of activatable antibody 5342-1204-4D11 and antibody 4D11 was observed by the increased shift in MW of the HC and LC under reduced conditions by SDS analysis. HIC-HPLC analysis demonstrated between 40 to 75% yield of toxin-conjugated material.

FIG. 9B demonstrates the potent killing activity of activatable antibody conjugate 5342-1204-4D11-vc-MMAD upon activation with uPA and antibody conjugate 4D11-vc-MMAD. Non-activated activatable antibody conjugate 5342-1204-4D11-vc-MMAD killed BxPC3 cells at a 17-fold reduced potency (IC50=7 nM) compared to antibody conjugate 4D11-vc-MMAD (IC50=0.4 nM). The lack of cell killing activity of rituximab antibody (an unrelated IgG1 isotype antibody) conjugated with the vc-MMAD linker is also shown. Antibody 4D11 alone (i.e., not conjugated) did not exhibit any cytotoxic activity on BxPC3.

### Example 9. Use of Activatable Antibody Conjugates that Include Microtubule Inhibitor Monomethyl Auristatin E (MMAE)

This example demonstrates that activatable antibody conjugates comprising microtubule inhibitor MMAE display potent in vitro killing activity. The example also demonstrates that addition of the linker payload to the activatable antibody does not interfere with the ability of the masking moiety to block binding of the activatable antibody comprising such masking moiety to its target.

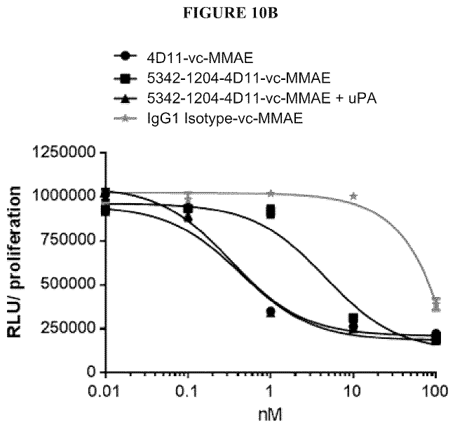

FIG. 10A demonstrates that partial reduction and subsequent thiol conjugation of anti-Jagged activatable antibody 5342-1204-4D11 or anti-Jagged antibody 4D11 with the vc-MMAE linker payload did not affect the binding behavior of the resultant conjugates to BxPC3 cells: There was a 33-fold reduction in IC50 of binding of activatable antibody conjugate 5342-1204-4D11-vc-MMAE (˜30 nM) (which was similar to the binding activity of non-conjugated activatable antibody 5342-1404-4D11) compared to antibody conjugate 4D11-vc-MMAE (0.9 nM). Upon activation with uPA, binding of activated activatable antibody conjugate 5342-1204-4D11-vc-MMAE was comparable to that of antibody conjugate 4D11-vc-MMAE. The figure also demonstrates that Synagis conjugated to vc-MMAE did not bind to BxPC3 cells.

FIG. 10B demonstrates the potent killing activity of activatable antibody conjugate 5342-1204-4D11-vc-MMAE upon activation with uPA and antibody conjugate 4D11-vc-MMAE. Non-activated activatable antibody conjugate 5342-1204-4D11-vc-MMAE killed BxPC3 cells at a 12-fold reduced potency (IC50=5 nM) compared to antibody conjugate 4D11-vc-MMAE (IC50=0.4 nM). The lack of cell killing activity of Synagis conjugated with the vc-MMAE linker is also shown.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following.

What is claimed is:

1. A method of selectively conjugating an agent to a thiol on an activatable antibody, the method comprising:
a) contacting an activatable antibody having a masking moiety (MM) conjugated thereto with tris(2-carboxyethyl) phosphine (TCEP) at a ratio of TCEP to the activatable antibody of less than 5:1 for 30 minutes to 2 hours, wherein the activatable antibody comprises at least one intrachain disulfide bond within the MM;
b) selectively reducing at least one interchain disulfide bond in the activatable antibody to form at least one thiol without reducing any intrachain disulfide bonds within the MM in the activatable antibody; and
c) conjugating an agent to the at least one thiol of the activatable antibody.

2. The method of claim 1, wherein the at least one disulfide bond is a disulfide bond between the activatable antibody and a second molecule.

3. The method of claim 2, wherein the second molecule is cysteine or glutathione.

4. The method of claim 1, wherein the activatable antibody in the uncleaved state has the structural arrangement from N-terminus to C-terminus as follows: MM-CM-AB or AB-CM-MM or wherein the activatable antibody in the uncleaved state comprises a spacer that is joined directly to the MM and has the structural arrangement from N-terminus to C-terminus of spacer-MM-CM-AB.

5. The method of claim 1, wherein the agent is a toxin or fragment thereof.

6. The method of claim 5, wherein the agent is a microtubule inhibitor or a nucleic acid damaging agent.

7. The method of claim 6, wherein the agent is a dolastatin or a derivative thereof, an auristatin or a derivative thereof, a maytansinoid or a derivative thereof, a duocarmycin or a derivative thereof, or a calicheamicin or a derivative thereof.

8. The method of claim 1, wherein the agent is auristatin E or a derivative thereof.

9. The method of claim 1, wherein the agent is monomethyl auristatin E (MMAE).

10. The method of claim 1, wherein the agent is monomethyl auristatin D (MMAD).

11. The method of claim 1, wherein the agent is DM1 or DM4.

12. The method of claim 1, wherein the agent is conjugated to the AB via a linker.

13. The method of claim 12, wherein the linker is a cleavable linker.

14. The method of claim 1, wherein the agent is a detectable moiety.

15. The method of claim 14, wherein the detectable moiety is a diagnostic agent.

16. The method of claim 1, wherein the antigen binding fragment thereof is selected from the group consisting of a Fab fragment, a F(ab′)₂ fragment, a scFv, a scAb, a dAb, a single domain heavy chain antibody, and a single domain light chain antibody.

17. The method of claim 1, wherein the MM has an equilibrium dissociation constant for binding to the AB that is greater than the equilibrium dissociation constant of the AB to the target.

18. The method of claim 1, wherein the MM does not interfere or compete with the AB for binding to the target when the activatable antibody is in a cleaved state.

19. The method of claim 1, wherein the MM is a polypeptide of no more than 40 amino acids in length.

20. The method of claim 1, wherein the CM is a substrate for one or more enzymes selected from the group consisting of: a urokinase plasminogen activator (uPA), a legumain, a matriptase, a matrix metalloprotease (MMP).


SEQUENCE LISTING (318 sequences)

SEQ ID NO: 1 - DNA, 1350 bp, synthetic construct
  misc_feature 1..1350: note = C225v5 Antibody Heavy Chain
  source 1..1350: mol_type = other DNA
caggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgaactgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 449 aa, synthetic construct
  REGION 1..449: note = C225v5 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 786 bp, synthetic construct
  misc_feature 1..786: note = 3954-1204-C225v5 Activatable Antibody Light Chain
  source 1..786: mol_type = other DNA
caaggccagtctggccagtgcatctcacctcgtggttgtccggacggcccatacgtcatgtacggctcgagcggtggcagcggtggctctggtggatccggtctgagcggccgttccgataatcatggcagtagcggtacccagatcttgctgacccagagcccggtgattctgagcgtgagcccgggcgaacgtgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 261 aa, synthetic construct
  REGION 1..261: note = 3954-1204-C225v5 Activatable Antibody Light Chain
QGQSGQCISPRGCPDGPYVMYGSSGGSGGSGGSGLSGRSDNHGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 18 bp, synthetic construct
  misc_feature 1..18: note = spacer peptide encoding sequence
  source 1..18: mol_type = other DNA
caaggccagtctggccag

SEQ ID NO: 6 - DNA, 45 bp, synthetic construct
  misc_feature 1..45: note = masking moiety encoding sequence
  source 1..45: mol_type = other DNA
tgcatctcacctcgtggttgtccggacggcccatacgtcatgtac

SEQ ID NO: 7 - DNA, 39 bp, synthetic construct
  misc_feature 1..39: note = linker peptide encoding sequence
  source 1..39: mol_type = other DNA
ggctcgagcggtggcagcggtggctctggtggatccggt

SEQ ID NO: 8 - DNA, 24 bp, synthetic construct
  misc_feature 1..24: note = 1204 substrate encoding sequence
  source 1..24: mol_type = other DNA
ctgagcggccgttccgataatcat

SEQ ID NO: 9 - DNA, 15 bp, synthetic construct
  misc_feature 1..15: note = linker peptide encoding sequence
  source 1..15: mol_type = other DNA
ggcagtagcggtacc

SEQ ID NO: 10 - DNA, 645 bp, synthetic construct
  misc_feature 1..645: note = C225v5 Antibody Light Chain
  source 1..645: mol_type = other DNA
cagatcttgctgacccagagcccggtgattctgagcgtgagcccgggcgaacgtgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 11 - protein, 6 aa, synthetic construct
  REGION 1..6: note = spacer peptide
QGQSGQ

SEQ ID NO: 12 - protein, 15 aa, synthetic construct
  REGION 1..15: note = masking moiety
CISPRGCPDGPYVMY

SEQ ID NO: 13 - protein, 13 aa, synthetic construct
  REGION 1..13: note = linker peptide
GSSGGSGGSGGSG

SEQ ID NO: 14 - protein, 8 aa, synthetic construct
  REGION 1..8: note = 1204 substrate
LSGRSDNH

SEQ ID NO: 15 - protein, 5 aa, synthetic construct
  REGION 1..5: note = linker peptide
GSSGT

SEQ ID NO: 16 - protein, 214 aa, synthetic construct
  REGION 1..214: note = C225v5 Antibody Light Chain
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 777 bp, synthetic construct
  misc_feature 1..777: note = 3954-NSUB-C225v5 Masked Antibody Light Chain
  source 1..777: mol_type = other DNA
caaggccagtctggccagtgcatctcacctcgtggttgtccggacggcccatacgtcatgtacggctcgagcggtggcagcggtggctctggtggctcaggtggaggctcgggcggtgggagcggcggttctcagatcttgctgacccagagcccggtgattctgagcgtgagcccgggcgaacgtgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - protein, 258 aa, synthetic construct
  REGION 1..258: note = 3954-NSUB-C225v5 Masked Antibody Light Chain
QGQSGQCISPRGCPDGPYVMYGSSGGSGGSGGSGGGSGGGSGGSQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 69 bp, synthetic construct
  misc_feature 1..69: note = linker - non-cleavable substrate - linker peptide encoding sequence
  source 1..69: mol_type = other DNA
ggctcgagcggtggcagcggtggctctggtggctcaggtggaggctcgggcggtgggagcggcggttct

SEQ ID NO: 20 - protein, 23 aa, synthetic construct
  REGION 1..23: note = linker - non-cleavable substrate - linker peptide
GSSGGSGGSGGSGGGSGGGSGGS

SEQ ID NO: 21 - protein, 5 aa, synthetic construct
  REGION 1..5: note = linking peptide
  REPEAT 1..5: note = May be repeated
GSGGS

SEQ ID NO: 22 - protein, 4 aa, synthetic construct
  REGION 1..4: note = linking peptide
  REPEAT 1..4: note = May be repeated
GGGS

SEQ ID NO: 23 - protein, 4 aa, synthetic construct
  REGION 1..4: note = linking peptide
GGSG

SEQ ID NO: 24 - protein, 5 aa, synthetic construct
  REGION 1..5: note = linking peptide
GGSGG

SEQ ID NO: 25 - protein, 5 aa, synthetic construct
  REGION 1..5: note = linking peptide
GSGSG

SEQ ID NO: 26 - protein, 5 aa, synthetic construct
  REGION 1..5: note = linking peptide
GSGGG

SEQ ID NO: 27 - protein, 5 aa, synthetic construct
  REGION 1..5: note = linking peptide
GGGSG

SEQ ID NO: 28 - protein, 5 aa, synthetic construct
  REGION 1..5: note = linking peptide
GSSSG

SEQ ID NO: 29 - protein, 6 aa, synthetic construct
  REGION 1..6: note = masking moiety
CISPRG

SEQ ID NO: 30 - protein, 8 aa, synthetic construct
  REGION 1..8: note = masking moiety
CISPRGCG

SEQ ID NO: 31 - protein, 15 aa, synthetic construct
  REGION 1..15: note = masking moiety
CISPRGCPDGPYVMY

SEQ ID NO: 32 - protein, 14 aa, synthetic construct
  REGION 1..14: note = masking moiety
CISPRGCPDGPYVM

SEQ ID NO: 33 - protein, 15 aa, synthetic construct
  REGION 1..15: note = masking moiety
CISPRGCEPGTYVPT

SEQ ID NO: 34 - protein, 15 aa, synthetic construct
  REGION 1..15: note = masking moiety
CISPRGCPGQIWHPP

SEQ ID NO: 35 - protein, 15 aa, synthetic construct
  REGION 1..15: note = masking moiety
GSHCLIPINMGAPSC

SEQ ID NO: 36 - protein, 32 aa, synthetic construct
  REGION 1..32: note = masking moiety
CISPRGCGGSSASQSGQGSHCLIPINMGAPSC

SEQ ID NO: 37 - protein, 19 aa, synthetic construct
  REGION 1..19: note = masking moiety
CNHHYFYTCGCISPRGCPG

SEQ ID NO: 38 - protein, 19 aa, synthetic construct
  REGION 1..19: note = masking moiety
ADHVFWGSYGCISPRGCPG

SEQ ID NO: 39 - protein, 19 aa, synthetic construct
  REGION 1..19: note = masking moiety
CHHVYWGHCGCISPRGCPG

SEQ ID NO: 40 - protein, 19 aa, synthetic construct
  REGION 1..19: note = masking moiety
CPHFTTTSCGCISPRGCPG

SEQ ID NO: 41 - protein, 19 aa, synthetic construct
  REGION 1..19: note = masking moiety
CNHHYHYYCGCISPRGCPG

SEQ ID NO: 42 - protein, 19 aa, synthetic construct
  REGION 1..19: note = masking moiety
CPHVSFGSCGCISPRGCPG

SEQ ID NO: 43 - protein, 19 aa, synthetic construct
  REGION 1..19: note = masking moiety
CPYYTLSYCGCISPRGCPG

SEQ ID NO: 44 - protein, 19 aa, synthetic construct
  REGION 1..19: note = masking moiety
CNHVYFGTCGCISPRGCPG

SEQ ID NO: 45 - protein, 19 aa, synthetic construct
  REGION 1..19: note = masking moiety
CNHFTLTTCGCISPRGCPG

SEQ ID NO: 46 - protein, 19 aa, synthetic construct
  REGION 1..19: note = masking moiety
CHHFTLTTCGCISPRGCPG

SEQ ID NO: 47 - protein, 18 aa, synthetic construct
  REGION 1..18: note = masking moiety
YNPCATPMCCISPRGCPG

SEQ ID NO: 48 - protein, 18 aa, synthetic construct
  REGION 1..18: note = masking moiety
CNHHYFYTCGCISPRGCG

SEQ ID NO: 49 - protein, 18 aa, synthetic construct
  REGION 1..18: note = masking moiety
CNHHYHYYCGCISPRGCG

SEQ ID NO: 50 - protein, 18 aa, synthetic construct
  REGION 1..18: note = masking moiety
CNHVYFGTCGCISPRGCG

SEQ ID NO: 51 - protein, 18 aa, synthetic construct
  REGION 1..18: note = masking moiety
CHHVYWGHCGCISPRGCG

SEQ ID NO: 52 - protein, 18 aa, synthetic construct
  REGION 1..18: note = masking moiety
CPHFTTTSCGCISPRGCG

SEQ ID NO: 53 - protein, 18 aa, synthetic construct
  REGION 1..18: note = masking moiety
CNHFTLTTCGCISPRGCG

SEQ ID NO: 54 - protein, 18 aa, synthetic construct
  REGION 1..18: note = masking moiety
CHHFTLTTCGCISPRGCG

SEQ ID NO: 55 - protein, 18 aa, synthetic construct
  REGION 1..18: note = masking moiety
CPYYTLSYCGCISPRGCG

SEQ ID NO: 56 - protein, 18 aa, synthetic construct
  REGION 1..18: note = masking moiety
CPHVSFGSCGCISPRGCG

SEQ ID NO: 57 - protein, 18 aa, synthetic construct
  REGION 1..18: note = masking moiety
ADHVFWGSYGCISPRGCG

SEQ ID NO: 58 - protein, 17 aa, synthetic construct
  REGION 1..17: note = masking moiety
YNPCATPMCCISPRGCG

SEQ ID NO: 59 - protein, 18 aa, synthetic construct
  REGION 1..18: note = masking moiety
CHHVYWGHCGCISPRGCG

SEQ ID NO: 60 - protein, 18 aa, synthetic construct
  REGION 1..18: note = masking moiety
  SITE 2: note = MISC_FEATURE - Xaa may be Asn or Phe
  SITE 4: note = MISC_FEATURE - Xaa may be His, Val or Phe
  SITE 5: note = MISC_FEATURE - Xaa may be Tyr or Thr
  SITE 6: note = MISC_FEATURE - Xaa may be Phe, Trp, Thr or Leu
  SITE 7: note = MISC_FEATURE - Xaa may be Tyr, Gly, Thr or Ser
  SITE 8: note = MISC_FEATURE - Xaa may be Thr, Ser, Tyr or His
CXHXXXXXCGCISPRGCG

SEQ ID NO: 61 - protein, 15 aa, synthetic construct
  REGION 1..15: note = masking moiety
CISPRGCGQPIPSVK

SEQ ID NO: 62 - protein, 15 aa, synthetic construct
  REGION 1..15: note = masking moiety
CISPRGCTQPYHVSR

SEQ ID NO: 63 - protein, 15 aa, synthetic construct
  REGION 1..15: note = masking moiety
CISPRGCNAVSGLGS

SEQ ID NO: 64 - protein, 26 aa, synthetic construct
  REGION 1..26: note = masking moiety
QGQSGQGQQQWCNIWINGGDCRGWNG

SEQ ID NO: 65 - protein, 15 aa, synthetic construct
  REGION 1..15: note = masking moiety
PWCMQRQDFLRCPQP

SEQ ID NO: 66 - protein, 15 aa, synthetic construct
  REGION 1..15: note = masking moiety
QLGLPAYMCTFECLR

SEQ ID NO: 67 - protein, 15 aa, synthetic construct
  REGION 1..15: note = masking moiety
CNLWVSGGDCGGLQG

SEQ ID NO: 68 - protein, 15 aa, synthetic construct
  REGION 1..15: note = masking moiety
SCSLWTSGSCLPHSP

SEQ ID NO: 69 - protein, 15 aa, synthetic construct
  REGION 1..15: note = masking moiety
YCLQLPHYMQAMCGR

SEQ ID NO: 70 - protein, 15 aa, synthetic construct
  REGION 1..15: note = masking moiety
CFLYSCTDVSYWNNT

SEQ ID NO: 71 - protein, 15 aa, synthetic construct
  REGION 1..15: note = masking moiety
PWCMQRQDYLRCPQP

SEQ ID NO: 72 - protein, 15 aa, synthetic construct
  REGION 1..15: note = masking moiety
CNLWISGGDCRGLAG

SEQ ID NO: 73 - protein, 15 aa, synthetic construct
  REGION 1..15: note = masking moiety
CNLWVSGGDCRGVQG

SEQ ID NO: 74 - protein, 15 aa, synthetic construct
  REGION 1..15: note = masking moiety
CNLWVSGGDCRGLRG

SEQ ID NO: 75 - protein, 15 aa, synthetic construct
  REGION 1..15: note = masking moiety
CNLWISGGDCRGLPG

SEQ ID NO: 76 - protein, 15 aa, synthetic construct
  REGION 1..15: note = masking moiety
CNLWVSGGDCRDAPW

SEQ ID NO: 77 - protein, 15 aa, synthetic construct
  REGION 1..15: note = masking moiety
CNLWVSGGDCRDLLG

SEQ ID NO: 78 - protein, 15 aa, synthetic construct
  REGION 1..15: note = masking moiety
CNLWVSGGDCRGLQG

SEQ ID NO: 79 - protein, 15 aa, synthetic construct
  REGION 1..15: note = masking moiety
CNLWLHGGDCRGWQG

SEQ ID NO: 80 - protein, 15 aa, synthetic construct
  REGION 1..15: note = masking moiety
CNIWLVGGDCRGWQG

SEQ ID NO: 81 - protein, 15 aa, synthetic construct
  REGION 1..15: note = masking moiety
CTTWFCGGDCGVMRG

SEQ ID NO: 82 - protein, 15 aa, synthetic construct
  REGION 1..15: note = masking moiety
CNIWGPSVDCGALLG

SEQ ID NO: 83 - protein, 15 aa, synthetic construct
  REGION 1..15: note = masking moiety
CNIWVNGGDCRSFEG

SEQ ID NO: 84 - protein, 15 aa, synthetic construct
  REGION 1..15: note = masking moiety
YCLNLPRYMQDMCWA

SEQ ID NO: 85 - protein, 15 aa, synthetic construct
  REGION 1..15: note = masking moiety
YCLALPHYMQADCAR

SEQ ID NO: 86 - protein, 15 aa, synthetic construct
  REGION 1..15: note = masking moiety
CFLYSCGDVSYWGSA

SEQ ID NO: 87 - protein, 15 aa, synthetic construct
  REGION 1..15: note = masking moiety
CYLYSCTDSAFWNNR

SEQ ID NO: 88 - protein, 15 aa, synthetic construct
  REGION 1..15: note = masking moiety
CYLYSCNDVSYWSNT

SEQ ID NO: 89 - protein, 12 aa, synthetic construct
  REGION 1..12: note = masking moiety
CFLYSCTDVSYW

SEQ ID NO: 90 - protein, 15 aa, synthetic construct
  REGION 1..15: note = masking moiety
CFLYSCTDVAYWNSA

SEQ ID NO: 91 - protein, 15 aa, synthetic construct
  REGION 1..15: note = masking moiety
CFLYSCTDVSYWGDT

SEQ ID NO: 92 - protein, 15 aa, synthetic construct
  REGION 1..15: note = masking moiety
CFLYSCTDVSYWGNS

SEQ ID NO: 93 - protein, 15 aa, synthetic construct
  REGION 1..15: note = masking moiety
CFLYSCTDVAYWNNT

SEQ ID NO: 94 - protein, 18 aa, synthetic construct
  REGION 1..18: note = masking moiety
CFLYSCGDVSYWGNPGLS

SEQ ID NO: 95 - protein, 15 aa, synthetic construct
  REGION 1..15: note = masking moiety
CFLYSCTDVAYWSGL

SEQ ID NO: 96 - protein, 15 aa, synthetic construct
  REGION 1..15: note = masking moiety
CYLYSCTDGSYWNST

SEQ ID NO: 97 - protein, 15 aa, synthetic construct
  REGION 1..15: note = masking moiety
CFLYSCSDVSYWGNI

SEQ ID NO: 98 - protein, 12 aa, synthetic construct
  REGION 1..12: note = masking moiety
CFLYSCTDVAYW

SEQ ID NO: 99 - protein, 15 aa, synthetic construct
  REGION 1..15: note = masking moiety
CFLYSCTDVSYWGST

SEQ ID NO: 100 - protein, 15 aa, synthetic construct
  REGION 1..15: note = masking moiety
CFLYSCTDVAYWGDT

SEQ ID NO: 101 - protein, 20 aa, synthetic construct
  REGION 1..20: note = masking moiety
GCNIWLNGGDCRGWVDPLQG

SEQ ID NO: 102 - protein, 20 aa, synthetic construct
  REGION 1..20: note = masking moiety
GCNIWLVGGDCRGWIGDTNG

SEQ ID NO: 103 - protein, 20 aa, synthetic construct
  REGION 1..20: note = masking moiety
GCNIWLVGGDCRGWIEDSNG

SEQ ID NO: 104 - protein, 20 aa, synthetic construct
  REGION 1..20: note = masking moiety
GCNIWANGGDCRGWIDNIDG

SEQ ID NO: 105 - protein, 20 aa, synthetic construct
  REGION 1..20: note = masking moiety
GCNIWLVGGDCRGWLGEAVG

SEQ ID NO: 106 - protein, 20 aa, synthetic construct
  REGION 1..20: note = masking moiety
GCNIWLVGGDCRGWLEEAVG

SEQ ID NO: 107 - protein, 20 aa, synthetic construct
  REGION 1..20: note = masking moiety
GGPALCNIWLNGGDCRGWSG

SEQ ID NO: 108 - protein, 20 aa, synthetic construct
  REGION 1..20: note = masking moiety
GAPVFCNIWLNGGDCRGWMG

SEQ ID NO: 109 - protein, 20 aa, synthetic construct
  REGION 1..20: note = masking moiety
GQQQWCNIWINGGDCRGWNG

SEQ ID NO: 110 - protein, 20 aa, synthetic construct
  REGION 1..20: note = masking moiety
GKSEFCNIWLNGGDCRGWIG

SEQ ID NO: 111 - protein, 20 aa, synthetic construct
  REGION 1..20: note = masking moiety
GTPGGCNIWANGGDCRGWEG

SEQ ID NO: 112 - protein, 20 aa, synthetic construct
  REGION 1..20: note = masking moiety
GASQYCNLWINGGDCRGWRG

SEQ ID NO: 113 - protein, 18 aa, synthetic construct
  REGION 1..18: note = masking moiety
GCNIWLVGGDCRPWVEGG

SEQ ID NO: 114 - protein, 18 aa, synthetic construct
  REGION 1..18: note = masking moiety
GCNIWAVGGDCRPFVDGG

SEQ ID NO: 115 - protein, 18 aa, synthetic construct
  REGION 1..18: note = masking moiety
GCNIWLNGGDCRAWVDTG

SEQ ID NO: 116 - protein, 18 aa, synthetic construct
  REGION 1..18: note = masking moiety
GCNIWIVGGDCRPFINDG

SEQ ID NO: 117 - protein, 18 aa, synthetic construct
  REGION 1..18: note = masking moiety
GCNIWLNGGDCRPVVFGG

SEQ ID NO: 118 - protein, 18 aa, synthetic construct
  REGION 1..18: note = masking moiety
GCNIWLSGGDCRMFMNEG

SEQ ID NO: 119 - protein, 18 aa, synthetic construct
  REGION 1..18: note = masking moiety
GCNIWVNGGDCRSFVYSG

SEQ ID NO: 120 - protein, 18 aa, synthetic construct
  REGION 1..18: note = masking moiety
GCNIWLNGGDCRGWEASG

SEQ ID NO: 121 - protein, 18 aa, synthetic construct
  REGION 1..18: note = masking moiety
GCNIWAHGGDCRGFIEPG

SEQ ID NO: 122 - protein, 18 aa, synthetic construct
  REGION 1..18: note = masking moiety
GCNIWLNGGDCRTFVASG

SEQ ID NO: 123 - protein, 18 aa, synthetic construct
  REGION 1..18: note = masking moiety
GCNIWAHGGDCRGFIEPG

SEQ ID NO: 124 - protein, 18 aa, synthetic construct
  REGION 1..18: note = masking moiety
GFLENCNIWLNGGDCRTG

SEQ ID NO: 125 - protein, 18 aa, synthetic construct
  REGION 1..18: note = masking moiety
GIYENCNIWLNGGDCRMG

SEQ ID NO: 126 - protein, 18 aa, synthetic construct
  REGION 1..18: note = masking moiety
GIPDNCNIWINGGDCRYG

SEQ ID NO: 127 - protein, 21 aa, synthetic construct
  REGION 1..21: note = masking moiety
QGQSGQYGSCSWNYVHIFMDC

SEQ ID NO: 128 - protein, 21 aa, synthetic construct
  REGION 1..21: note = masking moiety
QGQSGQGDFDIPFPAHWVPIT

SEQ ID NO: 129 - protein, 24 aa, synthetic construct
  REGION 1..24: note = masking moiety
QGQSGQMGVPAGCVWNYAHIFMDC

SEQ ID NO: 130 - protein, 15 aa, synthetic construct
  REGION 1..15: note = masking moiety
YRSCNWNYVSIFLDC

SEQ ID NO: 131 - protein, 16 aa, synthetic construct
  REGION 1..16: note = masking moiety
PGAFDIPFPAHWVPNT

SEQ ID NO: 132 - protein, 15 aa, synthetic construct
  REGION 1..15: note = masking moiety
ESSCVWNYVHIYMDC

SEQ ID NO: 133 - protein, 15 aa, synthetic construct
  REGION 1..15: note = masking moiety
YPGCKWNYDRIFLDC

SEQ ID NO: 134 - protein, 15 aa, synthetic construct
  REGION 1..15: note = masking moiety
YRTCSWNYVGIFLDC

SEQ ID NO: 135 - protein, 15 aa, synthetic construct
  REGION 1..15: note = masking moiety
YGSCSWNYVHIFMDC

SEQ ID NO: 136 - protein, 15 aa, synthetic construct
  REGION 1..15: note = masking moiety
YGSCSWNYVHIFLDC

SEQ ID NO: 137 - protein, 15 aa, synthetic construct
  REGION 1..15: note = masking moiety
YGSCNWNYVHIFLDC

SEQ ID NO: 138 - protein, 15 aa, synthetic construct
  REGION 1..15: note = masking moiety
YTSCNWNYVHIFMDC

SEQ ID NO: 139 - protein, 15 aa, synthetic construct
  REGION 1..15: note = masking moiety
YPGCKWNYDRIFLDC

SEQ ID NO: 140 - protein, 15 aa, synthetic construct
  REGION 1..15: note = masking moiety
WRSCNWNYAHIFLDC

SEQ ID NO: 141 - protein, 15 aa, synthetic construct
  REGION 1..15: note = masking moiety
WSNCHWNYVHIFLDC

SEQ ID NO: 142 - protein, 15 aa, synthetic construct
  REGION 1..15: note = masking moiety
DRSCTWNYVRISYDC

SEQ ID NO: 143 - protein, 15 aa, synthetic construct
  REGION 1..15: note = masking moiety
SGSCKWDYVHIFLDC

SEQ ID NO: 144 - protein, 15 aa, synthetic construct
  REGION 1..15: note = masking moiety
SRSCIWNYAHIHLDC

SEQ ID NO: 145 - protein, 15 aa, synthetic construct
  REGION 1..15: note = masking moiety
SMSCYWQYERIFLDC

SEQ ID NO: 146 - protein, 15 aa, synthetic construct
  REGION 1..15: note = masking moiety
YRSCNWNYVSIFLDC

SEQ ID NO: 147 - protein, 15 aa, synthetic construct
  REGION 1..15: note = masking moiety
YGSCSWNYVHIFMDC

SEQ ID NO: 148 - protein, 15 aa, synthetic construct
  REGION 1..15: note = masking moiety
SGSCKWDYVHIFLDC

SEQ ID NO: 149 - protein, 15 aa, synthetic construct
  REGION 1..15: note = masking moiety
YKSCHWDYVHIFLDC

SEQ ID NO: 150 - protein, 15 aa, synthetic construct
  REGION 1..15: note = masking moiety
YGSCTWNYVHIFMEC

SEQ ID NO: 151 - protein, 15 aa, synthetic construct
  REGION 1..15: note = masking moiety
FSSCNWNYVHIFLDC

SEQ ID NO: 152 - protein, 15 aa, synthetic construct
  REGION 1..15: note = masking moiety
WRSCNWNYAHIFLDC

SEQ ID NO: 153 - protein, 15 aa, synthetic construct
  REGION 1..15: note = masking moiety
YGSCQWNYVHIFLDC

SEQ ID NO: 154 - protein, 15 aa, synthetic construct
  REGION 1..15: note = masking moiety
YRSCNWNYVHIFLDC

SEQ ID NO: 155 - protein, 15 aa, synthetic construct
  REGION 1..15: note = masking moiety
NMSCHWDYVHIFLDC

SEQ ID NO: 156 - protein, 15 aa, synthetic construct
  REGION 1..15: note = masking moiety
FGPCTWNYARISWDC

SEQ ID NO: 157 - protein, 15 aa, synthetic construct
  REGION 1..15: note = masking moiety
  REGION 1..2: note = misc_feature - Xaa can be any naturally occurring amino acid
  SITE 5: note = misc_feature - Xaa can be any naturally occurring amino acid
  SITE 7: note = misc_feature - Xaa can be any naturally occurring amino acid
  SITE 13: note = misc_feature - Xaa can be any naturally occurring amino acid
XXSCXWXYVHIFXDC

SEQ ID NO: 158 - protein, 18 aa, synthetic construct
  REGION 1..18: note = masking moiety
MGVPAGCVWNYAHIFMDC

SEQ ID NO: 159 - protein, 18 aa, synthetic construct
  REGION 1..18: note = masking moiety
RDTGGQCRWDYVHIFMDC

SEQ ID NO: 160 - protein, 18 aa, synthetic construct
  REGION 1..18: note = masking moiety
AGVPAGCTWNYVHIFMEC

SEQ ID NO: 161 - protein, 18 aa, synthetic construct
  REGION 1..18: note = masking moiety
VGVPNGCVWNYAHIFMEC

SEQ ID NO: 162 - protein, 18 aa, synthetic construct
  REGION 1..18: note = masking moiety
DGGPAGCSWNYVHIFMEC

SEQ ID NO: 163 - protein, 18 aa, synthetic construct
  REGION 1..18: note = masking moiety
AVGPAGCWWNYVHIFMEC

SEQ ID NO: 164 - protein, 18 aa, synthetic construct
  REGION 1..18: note = masking moiety
CTWNYVHIFMDCGEGEGP

SEQ ID NO: 165 - protein, 18 aa, synthetic construct
  REGION 1..18: note = masking moiety
GGVPEGCTWNYAHIFMEC

SEQ ID NO: 166 - protein, 18 aa, synthetic construct
  REGION 1..18: note = masking moiety
AEVPAGCWWNYVHIFMEC

SEQ ID NO: 167 - protein, 18 aa, synthetic construct
  REGION 1..18: note = masking moiety
AGVPAGCTWNYVHIFMEC

SEQ ID NO: 168 - protein, 18 aa, synthetic construct
  REGION 1..18: note = masking moiety
SGASGGCKWNYVHIFMDC

SEQ ID NO: 169 - protein, 18 aa, synthetic construct
  REGION 1..18: note = masking moiety
MGVPAGCVWNYAHIFMDC

SEQ ID NO: 170 - protein, 18 aa, synthetic construct
  REGION 1..18: note = masking moiety
TPGCRWNYVHIFMECEAL

SEQ ID NO: 171 - protein, 18 aa, synthetic construct
  REGION 1..18: note = masking moiety
VGVPNGCVWNYAHIFMEC

SEQ ID NO: 172 - protein, 16 aa, synthetic construct
  REGION 1..16: note = masking moiety
PGAFDIPFPAHWVPNT

SEQ ID NO: 173 - protein, 16 aa, synthetic construct
  REGION 1..16: note = masking moiety
RGACDIPFPAHWIPNT

SEQ ID NO: 174 - protein, 16 aa, synthetic construct
  REGION 1..16: note = masking moiety
QGDFDIPFPAHWVPIT

SEQ ID NO: 175 - protein, 16 aa, synthetic construct
  REGION 1..16: note = masking moiety
  SITE 1: note = misc_feature - Xaa can be any naturally occurring amino acid
XGAFDIPFPAHWVPNT

SEQ ID NO: 176 - protein, 19 aa, synthetic construct
  REGION 1..19: note = masking moiety
RGDGNDSDIPFPAHWVPRT

SEQ ID NO: 177 - protein, 19 aa, synthetic construct
  REGION 1..19: note = masking moiety
SGVGRDRDIPFPAHWVPRT

SEQ ID NO: 178 - protein, 19 aa, synthetic construct
  REGION 1..19: note = masking moiety
WAGGNDCDIPFPAHWIPNT

SEQ ID NO: 179 - protein, 19 aa, synthetic construct
  REGION 1..19: note = masking moiety
WGDGMDVDIPFPAHWVPVT

SEQ ID NO: 180 - protein, 19 aa, synthetic construct
  REGION 1..19: note = masking moiety
AGSGNDSDIPFPAHWVPRT

SEQ ID NO: 181 - protein, 19 aa, synthetic construct
  REGION 1..19: note = masking moiety
ESRSGYADIPFPAHWVPRT

SEQ ID NO: 182 - protein, 19 aa, synthetic construct
  REGION 1..19: note = masking moiety
RECGRCGDIPFPAHWVPRT

SEQ ID NO: 183 - protein, 8 aa, synthetic construct
  REGION 1..8: note = cleavable moiety
TGRGPSWV

SEQ ID NO: 184 - protein, 8 aa, synthetic construct
  REGION 1..8: note = cleavable moiety
SARGPSRW

SEQ ID NO: 185 - protein, 8 aa, synthetic construct
  REGION 1..8: note = cleavable moiety
TARGPSFK

SEQ ID NO: 186 - protein, 8 aa, synthetic construct
  REGION 1..8: note = cleavable moiety
LSGRSDNH

SEQ ID NO: 187 - protein, 8 aa, synthetic construct
  REGION 1..8: note = cleavable moiety
GGWHTGRN

SEQ ID NO: 188 - protein, 8 aa, synthetic construct
  REGION 1..8: note = cleavable moiety
HTGRSGAL

SEQ ID NO: 189 - protein, 8 aa, synthetic construct
  REGION 1..8: note = cleavable moiety
PLTGRSGG

SEQ ID NO: 190 - protein, 8 aa, synthetic construct
  REGION 1..8: note = cleavable moiety
AARGPAIH

SEQ ID NO: 191 - protein, 8 aa, synthetic construct
  REGION 1..8: note = cleavable moiety
RGPAFNPM

SEQ ID NO: 192 - protein, 8 aa, synthetic construct
  REGION 1..8: note = cleavable moiety
SSRGPAYL

SEQ ID NO: 193 - protein, 8 aa, synthetic construct
  REGION 1..8: note = cleavable moiety
RGPATPIM

SEQ ID NO: 194 - protein, 4 aa, synthetic construct
  REGION 1..4: note = cleavable moiety
RGPA

SEQ ID NO: 195 - protein, 10 aa, synthetic construct
  REGION 1..10: note = cleavable moiety
GGQPSGMWGW

SEQ ID NO: 196 - protein, 10 aa, synthetic construct
  REGION 1..10: note = cleavable moiety
FPRPLGITGL

SEQ ID NO: 197 - protein, 10 aa, synthetic construct
  REGION 1..10: note = cleavable moiety
VHMPLGFLGP

SEQ ID NO: 198 - protein, 8 aa, synthetic construct
  REGION 1..8: note = cleavable moiety
SPLTGRSG

SEQ ID NO: 199 - protein, 8 aa, synthetic construct
  REGION 1..8: note = cleavable moiety
SAGFSLPA

SEQ ID NO: 200 - protein, 9 aa, synthetic construct
  REGION 1..9: note = cleavable moiety
LAPLGLQRR

SEQ ID NO: 201 - protein, 8 aa, synthetic construct
  REGION 1..8: note = cleavable moiety
SGGPLGVR

SEQ ID NO: 202 - protein, 4 aa, synthetic construct
  REGION 1..4: note = cleavable moiety
PLGL

SEQ ID NO: 203 - protein, 8 aa, synthetic construct
  REGION 1..8: note = cleavable linker
PRFKIIGG

SEQ ID NO: 204 - protein, 8 aa, synthetic construct
  REGION 1..8: note = cleavable linker
PRFRIIGG

SEQ ID NO: 205 - protein, 9 aa, synthetic construct
  REGION 1..9: note = cleavable linker
SSRHRRALD

SEQ ID NO: 206 - protein, 14 aa, synthetic construct
  REGION 1..14: note = cleavable linker
RKSSIIIRMRDVVL

SEQ ID NO: 207 - protein, 15 aa, synthetic construct
  REGION 1..15: note = cleavable linker
SSSFDKGKYKKGDDA

SEQ ID NO: 208 - protein, 15 aa, synthetic construct
  REGION 1..15: note = cleavable linker
SSSFDKGKYKRGDDA

SEQ ID NO: 209 - protein, 4 aa, synthetic construct
  REGION 1..4: note = cleavable linker
IEGR

SEQ ID NO: 210 - protein, 4 aa, synthetic construct
  REGION 1..4: note = cleavable linker
IDGR

SEQ ID NO: 211 - protein, 7 aa, synthetic construct
  REGION 1..7: note = cleavable linker
GGSIDGR

SEQ ID NO: 212 - protein, 6 aa, synthetic construct
  REGION 1..6: note = cleavable linker
PLGLWA

SEQ ID NO: 213 - protein, 8 aa, synthetic construct
  REGION 1..8: note = cleavable linker
GPQGIAGQ

SEQ ID NO: 214 - protein, 8 aa, synthetic construct
  REGION 1..8: note = cleavable linker
GPQGLLGA

SEQ ID NO: 215 - protein, 5 aa, synthetic construct
  REGION 1..5: note = cleavable linker
GIAGQ

SEQ ID NO: 216 - protein, 8 aa, synthetic construct
  REGION 1..8: note = cleavable linker
GPLGIAGI

SEQ ID NO: 217 - protein, 8 aa, synthetic construct
  REGION 1..8: note = cleavable linker
GPEGLRVG

SEQ ID NO: 218 - protein, 8 aa, synthetic construct
  REGION 1..8: note = cleavable linker
YGAGLGVV

SEQ ID NO: 219 - protein, 8 aa, synthetic construct
  REGION 1..8: note = cleavable linker
AGLGVVER

SEQ ID NO: 220 - protein, 8 aa, synthetic construct
  REGION 1..8: note = cleavable linker
AGLGISST

SEQ ID NO: 221 - protein, 8 aa, synthetic construct
  REGION 1..8: note = cleavable linker
EPQALAMS

SEQ ID NO: 222 - protein, 8 aa, synthetic construct
  REGION 1..8: note = cleavable linker
QALAMSAI

SEQ ID NO: 223 - protein, 8 aa, synthetic construct
  REGION 1..8: note = cleavable linker
AAYHLVSQ

SEQ ID NO: 224 - protein, 8 aa, synthetic construct
  REGION 1..8: note = cleavable linker
MDAFLESS

SEQ ID NO: 225 - protein, 8 aa, synthetic construct
  REGION 1..8: note = cleavable linker
ESLPVVAV

SEQ ID NO: 226 - protein, 8 aa, synthetic construct
  REGION 1..8: note = cleavable linker
SAPAVESE

SEQ ID NO: 227 - protein, 8 aa, synthetic construct
  REGION 1..8: note = cleavable linker
DVAQFVLT

SEQ ID NO: 228 - protein, 8 aa, synthetic construct
  REGION 1..8: note = cleavable linker
VAQFVLTE

SEQ ID NO: 229 - protein, 8 aa, synthetic construct
  REGION 1..8: note = cleavable linker
AQFVLTEG

SEQ ID NO: 230 - protein, 8 aa, synthetic construct
  REGION 1..8: note = cleavable linker
PVQPIGPQ

SEQ ID NO: 231 - DNA, 1347 bp, synthetic construct
  misc_feature 1..1347: note = 5342-1204-4D11 Activatable Antibody Heavy Chain
  source 1..1347: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtgtcaagtattgacccggaaggtcggcagacatattacgcagactccgtgaagggccggttcaccatctccagagacaattccaagaacacgctgtatctgcaaatgaacagcctgagagccgaggacacggccgtatattactgtgcgaaagacatcggcggcaggtcggcctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 232 - protein, 21 aa, synthetic construct
  REGION 1..21: note = masking moiety
QGQSGQCNIWLVGGDCRGWQG

SEQ ID NO: 233 - DNA, 774 bp, synthetic construct
  misc_feature 1..774: note = 5342-1204-4D11 Activatable Antibody Light Chain
  source 1..774: mol_type = other DNA
caaggccagtctggccagtgcaatatttggctcgtaggtggtgattgcaggggctggcaggggggctcgagcggtggcagcggtggctctggtggtctgagcggccgttccgataatcatggcggcggttctgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgcggcatccagtttgcaaagtggggtcccatcaaggttcagtggcagtggatctgggacagatttcactctcaccatcagcagtctgcaacctgaagattttgcaacttactactgtcaacagacggttgtggcgcctccgttattcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 234 - protein, 258 aa, synthetic construct
  REGION 1..258: note = 5342-1204-4D11 Activatable Antibody Light Chain
QGQSGQCNIWLVGGDCRGWQGGSSGGSGGSGGLSGRSDNHGGGSDIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 5 aa, synthetic construct
  REGION 1..5: note = spacer peptide
GQSGQ

SEQ ID NO: 236 - protein, 4 aa, synthetic construct
  REGION 1..4: note = spacer peptide
QSGQ

SEQ ID NO: 237 - skipped

SEQ ID NO: 238 - DNA, 1350 bp, synthetic construct
  misc_feature 1..1350: note = C225v4 Antibody Heavy Chain
  source 1..1350: mol_type = other DNA
caggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagcaacgataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgaactgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 239 - protein, 449 aa, synthetic construct
  REGION 1..449: note = C225v4 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - DNA, 1350 bp, synthetic construct
  misc_feature 1..1350: note = C225v6 Antibody Heavy Chain
  source 1..1350: mol_type = other DNA
caggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgaactgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 241 - protein, 449 aa, synthetic construct
  REGION 1..449: note = C225v6 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 449 aa, synthetic construct
  REGION 1..449: note = Av1 Antibody Heavy Chain
QVQLQESGPGLVRPSQTLSLTCTVSGYSITSDHAWSWVRQPPGRGLEWIGYISYSGITTYNPSLKSRVTISRDNSKNTLYLQMNSLRAEDTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 214 aa, synthetic construct [sequence omitted]

SEQ ID NO: 244 - protein, 214 aa, synthetic construct
  REGION 1..214: note = 4D11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 449 aa, synthetic construct
  REGION 1..449: note = 4D11 Heavy Chain
EVQLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 449 aa, synthetic construct
  REGION 1..449: note = 4D11v2 Heavy Chain
EVHLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 214 aa, synthetic construct
  REGION 1..214: note = 4D11v2 Light Chain
  SITE 182: note = misc_feature - Xaa can be any naturally occurring amino acid
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKRTVAAPSVFIFPPSDEQLKSGTASVVCLLNNFYPREAKVQWKVDNALQSGNSQESVTEQDSKDSTYSLSSTLTLXKADYEKHKVYACEVTHQGLSSPVTKSFNRGEC

SEQ ID NO: 248 - protein, 108 aa, synthetic construct
  REGION 1..108: note = Variable Light Chain Lc4
DIQMTQSPSSLSASVGDRVTITCRASQSISSYLNWYQQKPGKAPKLLIYAASSLQSGVPSRFSGSGSGTDFTLTISSLQPEDFATYYCQQSVVAPLTFGQGTKVEIKR

SEQ ID NO: 249 - protein, 119 aa, synthetic construct
  REGION 1..119: note = Variable Heavy Chain Hc4
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKDIGGRSAFDYWGQGTLVTVSS

SEQ ID NO: 250 - protein, 108 aa, synthetic construct; identical to SEQ ID NO: 248
  REGION 1..108: note = Variable Light Chain Lc5

SEQ ID NO: 251 - protein, 119 aa, synthetic construct
  REGION 1..119: note = Variable Heavy Chain Hc5
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPYHGQFDYWGQGTLVTVSS

SEQ ID NO: 252 - protein, 108 aa, synthetic construct; identical to SEQ ID NO: 248
  REGION 1..108: note = Variable Light Chain Lc7

SEQ ID NO: 253 - protein, 119 aa, synthetic construct
  REGION 1..119: note = Variable Heavy Chain Hc7
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPFFGQFDYWGQGTLVTVSS

SEQ ID NO: 254 - protein, 108 aa, synthetic construct; identical to SEQ ID NO: 248
  REGION 1..108: note = Variable Light Chain Lc8

SEQ ID NO: 255 - protein, 119 aa, synthetic construct
  REGION 1..119: note = Variable Heavy Chain Hc8
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKHIGRTNPFDYWGQGTLVTVSS

SEQ ID NO: 256 - protein, 108 aa, synthetic construct; identical to SEQ ID NO: 248
  REGION 1..108: note = Variable Light Chain Lc13

SEQ ID NO: 257 - protein, 116 aa, synthetic construct
  REGION 1..116: note = Variable Heavy Chain Hc13
EVQLLESGGGLVQPGGSLRLSCAASGFTFSSYAMSWVRQAPGKGLEWVSSIEQMGWQTEYADSVKGRFTISRDNSKNTLYLQMNSLRAEDTAVYYCAKSAAAFDYWGQGTLVTVSS

SEQ ID NO: 258 - protein, 108 aa, synthetic construct; identical to SEQ ID NO: 248
  REGION 1..108: note = Variable Light Chain Lc16

SEQ ID NO: 259 - protein, 119 aa, synthetic construct
  REGION 1..119: note = Variable Heavy Chain Hc16
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPYYGQFDYWGQGTLVTVSS

SEQ ID NO: 260 - protein, 108 aa, synthetic construct; identical to SEQ ID NO: 248
  REGION 1..108: note = Variable Light Chain Lc19

SEQ ID NO: 261 - protein, 119 aa, synthetic construct; identical to SEQ ID NO: 253
  REGION 1..119: note = Variable Heavy Chain Hc19

SEQ ID NO: 262 - protein, 108 aa, synthetic construct; identical to SEQ ID NO: 248
  REGION 1..108: note = Variable Light Chain Lc21

SEQ ID NO: 263 - protein, 119 aa, synthetic construct; identical to SEQ ID NO: 249
  REGION 1..119: note = Variable Heavy Chain Hc21

SEQ ID NO: 264 - protein, 108 aa, synthetic construct; identical to SEQ ID NO: 248
  REGION 1..108: note = Variable Light Chain Lc24

SEQ ID NO: 265 - protein, 116 aa, synthetic construct
  REGION 1..116: note = Variable Heavy Chain Hc24
EVQLLESGGGLVQPGGSLRLSCAASGFTFSSYAMSWVRQAPGKGLEWVSSIEEMGWQTLYADSVKGRFTISRDNSKNTLYLQMNSLRAEDTAVYYCAKSAAAFDYWGQGTLVTVSS

SEQ ID NO: 266 - protein, 108 aa, synthetic construct; identical to SEQ ID NO: 248
  REGION 1..108: note = Variable Light Chain Lc26

SEQ ID NO: 267 - protein, 119 aa, synthetic construct; identical to SEQ ID NO: 249
  REGION 1..119: note = Variable Heavy Chain Hc26

SEQ ID NO: 268 - protein, 108 aa, synthetic construct; identical to SEQ ID NO: 248
  REGION 1..108: note = Variable Light Chain Lc27

SEQ ID NO: 269 - protein, 119 aa, synthetic construct
  REGION 1..119: note = Variable Heavy Chain Hc27
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPFYGQFDYWGQGTLVTVSS

SEQ ID NO: 270 - protein, 108 aa, synthetic construct; identical to SEQ ID NO: 248
  REGION 1..108: note = Variable Light Chain Lc28

SEQ ID NO: 271 - protein, 119 aa, synthetic construct; identical to SEQ ID NO: 253
  REGION 1..119: note = Variable Heavy Chain Hc28

SEQ ID NO: 272 - protein, 108 aa, synthetic construct; identical to SEQ ID NO: 248
  REGION 1..108: note = Variable Light Chain Lc30

SEQ ID NO: 273 - protein, 115 aa, synthetic construct
  REGION 1..115: note = Variable Heavy Chain Hc30
EVQLLESGGGLVQPGGSLRLSCAASGFTFSSYAMSWVRQAPGKGLEWVSSIEEMGWQTLYADSVKGRFTISRDNSKNTLYLQMNSLRAEDTAVYYAKSAAAFDYWGQGTLVTVSS

SEQ ID NO: 274 - protein, 108 aa, synthetic construct; identical to SEQ ID NO: 248
  REGION 1..108: note = Variable Light Chain Lc31

SEQ ID NO: 275 - protein, 119 aa, synthetic construct; identical to SEQ ID NO: 249
  REGION 1..119: note = Variable Heavy Chain Hc31

SEQ ID NO: 276 - protein, 108 aa, synthetic construct; identical to SEQ ID NO: 248
  REGION 1..108: note = Variable Light Chain Lc32

SEQ ID NO: 277 - protein, 116 aa, synthetic construct
  REGION 1..116: note = Variable Heavy Chain Hc32
EVQLLESGGGLVQPGGSLRLSCAASGFTFSSYAMSWVRQAPGKGLEWVSSIDPEGWQTYYADSVKGRFTISRDNSKNTLYLQMNSLRAEDTAVYYCAKSAAAFDYWGQGTLVTVSS

SEQ ID NO: 278 - protein, 108 aa, synthetic construct; identical to SEQ ID NO: 248
  REGION 1..108: note = Variable Light Chain Lc37

SEQ ID NO: 279 - protein, 119 aa, synthetic construct
  REGION 1..119: note = Variable Heavy Chain Hc37
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPHNGQFDYWGQGTLVTVSS

SEQ ID NO: 280 - protein, 108 aa, synthetic construct; identical to SEQ ID NO: 248
  REGION 1..108: note = Variable Light Chain Lc39

SEQ ID NO: 281 - protein, 116 aa, synthetic construct; identical to SEQ ID NO: 257
  REGION 1..116: note = Variable Heavy Chain Hc39

SEQ ID NO: 282 - protein, 108 aa, synthetic construct; identical to SEQ ID NO: 248
  REGION 1..108: note = Variable Light Chain Lc40

SEQ ID NO: 283 - protein, 119 aa, synthetic construct; identical to SEQ ID NO: 253
  REGION 1..119: note = Variable Heavy Chain Hc40

SEQ ID NO: 284 - protein, 108 aa, synthetic construct; identical to SEQ ID NO: 248
  REGION 1..108: note = Variable Light Chain Lc47

SEQ ID NO: 285 - protein, 116 aa, synthetic construct
  REGION 1..116: note = Variable Heavy Chain Hc47
EVQLLESGGGLVQPGGSLRLSCAASGFTFSSYAMSWVRQAPGKGLEWVSSIDEMGWQTEYADSVKGRFTISRDNSKNTLYLQMNSLRAEDTAVYYCAKSAAAFDYWGQGTLVTVSS

SEQ ID NO: 286 - protein, 108 aa, synthetic construct
  REGION 1..108: note = Variable 4B2 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTLDAPPQFGQGTKVEIKR

SEQ ID NO: 287 - protein, 119 aa, synthetic construct; identical to SEQ ID NO: 249
  REGION 1..119: note = Variable 4B2 Heavy Chain

SEQ ID NO: 288 - protein, 108 aa, synthetic construct
  REGION 1..108: note = Variable 4D11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKR

SEQ ID NO: 289 - protein, 119 aa, synthetic construct
  REGION 1..119: note = Variable 4D11 Heavy Chain
EVQLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSS

SEQ ID NO: 290 - protein, 108 aa, synthetic construct
  REGION 1..108: note = Variable 4E7 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSLVAPLTFGQGTKVEIKR

SEQ ID NO: 291 - protein, 116 aa, synthetic construct
  REGION 1..116: note = Variable 4E7 Heavy Chain
EVQLLESGGGLVQPGGSLRLSCAASGFTFSSYAMSWVRQAPGKGLEWVSSIEEMGWQTKYADSVKGRFTISRDNSKNTLYLQMNSLRAEDTAVYYCAKSAAAFDYWGQGTLVTVSS

SEQ ID NO: 292 - protein, 108 aa, synthetic construct
  REGION 1..108: note = Variable 4E11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQALDAPLMFGQGTKVEIKR

SEQ ID NO: 293 - protein, 119 aa, synthetic construct
  REGION 1..119: note = Variable 4E11 Heavy Chain
EVQLLESGGGLVQPGGSLRLSCAASGFTFSSYAMSWVRQAPGKGLEWVSSIEPMGQLTEYADSVKGRFTISRDNSKNTLYLQMNSLRAEDTAVYYCAKDIGGRSAFDYWGQGTLVTVSS

SEQ ID NO: 294 - protein, 108 aa, synthetic construct
  REGION 1..108: note = Variable 6B7 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQALVAPLTFGQGTKVEIKR

SEQ ID NO: 295 - protein, 116 aa, synthetic construct
  REGION 1..116: note = Variable 6B7 Heavy Chain
EVQLLESGGGLVQPGGSLRLSCAASGFTFSSYAMSWVRQAPGKGLEWVSSIDEMGWQTYYADSVKGRFTISRDNSKNTLYLQMNSLRAEDTAVYYCAKSAAAFDYWGQGTLVTVSS

SEQ ID NO: 296 - protein, 108 aa, synthetic construct; identical to SEQ ID NO: 294
  REGION 1..108: note = Variable 6F8 Light Chain

SEQ ID NO: 297 - protein, 116 aa, synthetic construct; identical to SEQ ID NO: 295
  REGION 1..116: note = Variable 6F8 Heavy Chain

SEQ ID NO: 298 - protein, 8 aa, synthetic construct
  REGION 1..8: note = cleavable moiety
ISSGLLSS

SEQ ID NO: 299 - protein, 8 aa, synthetic construct
  REGION 1..8: note = cleavable moiety
QNQALRMA

SEQ ID NO: 300 - protein, 8 aa, synthetic construct
  REGION 1..8: note = cleavable moiety
AQNLLGMV

SEQ ID NO: 301 - protein, 8 aa, synthetic construct
  REGION 1..8: note = cleavable moiety
STFPFGMF

SEQ ID NO: 302 - protein, 8 aa, synthetic construct
  REGION 1..8: note = cleavable moiety
PVGYTSSL

SEQ ID NO: 303 - protein, 8 aa, synthetic construct
  REGION 1..8: note = cleavable moiety
DWLYWPGI

SEQ ID NO: 304 - protein, 8 aa, synthetic construct
  REGION 1..8: note = cleavable moiety
MIAPVAYR

SEQ ID NO: 305 - protein, 8 aa, synthetic construct
  REGION 1..8: note = cleavable moiety
RPSPMWAY

SEQ ID NO: 306 - protein, 8 aa, synthetic construct
  REGION 1..8: note = cleavable moiety
WATPRPMR

SEQ ID NO: 307 - protein, 8 aa, synthetic construct
  REGION 1..8: note = cleavable moiety
FRLLDWQW

SEQ ID NO: 308 - protein, 8 aa, synthetic construct
  REGION 1..8: note = cleavable moiety
LKAAPRWA

SEQ ID NO: 309 - protein, 8 aa, synthetic construct
  REGION 1..8: note = cleavable moiety
GPSHLVLT

SEQ ID NO: 310 - protein, 8 aa, synthetic construct
  REGION 1..8: note = cleavable moiety
LPGGLSPW

SEQ ID NO: 311 - protein, 8 aa, synthetic construct
  REGION 1..8: note = cleavable moiety
MGLFSEAG

SEQ ID NO: 312 - protein, 8 aa, synthetic construct
  REGION 1..8: note = cleavable moiety
SPLPLRVP

SEQ ID NO: 313 - protein, 8 aa, synthetic construct
  REGION 1..8: note = cleavable moiety
RMHLRSLG

SEQ ID NO: 314 - protein, 8 aa, synthetic construct
  REGION 1..8: note = cleavable moiety
LAAPLGLL

SEQ ID NO: 315 - protein, 8 aa, synthetic construct
  REGION 1..8: note = cleavable moiety
AVGLLAPP

SEQ ID NO: 316 - protein, 8 aa, synthetic construct
  REGION 1..8: note = cleavable moiety
LLAPSHRA

SEQ ID NO: 317 - protein, 8 aa, synthetic construct
  REGION 1..8: note = cleavable moiety
PAGLWLDP

SEQ ID NO: 318 - protein, 7 aa, synthetic construct
  REGION 1..7: note = cleavable moiety
ISSGLSS